SEQUENCE LISTING (48 sequences)

SEQ ID NO: 1 - protein, 122 aa, Artificial Sequence
  Description of Artificial Sequence Synthetic polypeptide
EVQLVESGGGLVQPGGSLRLSCAASGFTFSNFGMHWVRQAPGKGLEWVAYISSGGSSINYADTVKGRFTISRDNPKNSLYLQMNSLRAEDTAVYYCARGGTGTRSLYYFDYWGQGTLVTVSS

SEQ ID NO: 2 - protein, 5 aa, Artificial Sequence
  Description of Artificial Sequence Synthetic peptide
NFGMH

SEQ ID NO: 3 - protein, 7 aa, Artificial Sequence
  Description of Artificial Sequence Synthetic peptide
GFTFSNF

SEQ ID NO: 4 - protein, 17 aa, Artificial Sequence
  Description of Artificial Sequence Synthetic peptide
YISSGGSSINYADTVKG

SEQ ID NO: 5 - protein, 6 aa, Artificial Sequence
  Description of Artificial Sequence Synthetic peptide
SSGGSS

SEQ ID NO: 6 - protein, 13 aa, Artificial Sequence
  Description of Artificial Sequence Synthetic peptide
GGTGTRSLYYFDY

SEQ ID NO: 7 - protein, 452 aa, Artificial Sequence
  Description of Artificial Sequence Synthetic polypeptide
EVQLVESGGGLVQPGGSLRLSCAASGFTFSNFGMHWVRQAPGKGLEWVAYISSGGSSINYADTVKGRFTISRDNPKNSLYLQMNSLRAEDTAVYYCARGGTGTRSLYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - DNA, 1356 bp, Artificial Sequence
  Description of Artificial Sequence Synthetic polynucleotide
gaggtgcagctggtggagagcggcggcggcctggtgcagcccggcggcagcctgcggctgagctgcgccgccagcggcttcaccttcagcaacttcggcatgcactgggtgcggcaggcccccggcaagggcctggagtgggtggcctacatcagcagcggcggcagcagcatcaactacgccgacaccgtgaagggccggttcaccatcagccgggacaaccccaagaacagcctgtacctgcagatgaacagcctgcgggccgaggacaccgccgtgtactactgcgcccggggcggcaccggcacccggagcctgtactacttcgactactggggccagggcaccctggtgaccgtgtcctcagcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 9 - protein, 107 aa, Artificial Sequence
  Description of Artificial Sequence Synthetic polypeptide
DIQMTQSPSSLSASVGDRVTITCRASQDIGNFLNWYQQKPGKAPKLLIYYTSRLQSGVPSRFSGWGSGTDFTLTISSLQPEDFATYYCQQGKTLPYTFGGGTKVEIK

SEQ ID NO: 10 - protein, 11 aa, Artificial Sequence
  Description of Artificial Sequence Synthetic peptide
RASQDIGNFLN

SEQ ID NO: 11 - protein, 11 aa, Artificial Sequence
  Description of Artificial Sequence Synthetic peptide
RASQDIGNFLN

SEQ ID NO: 12 - protein, 7 aa, Artificial Sequence
  Description of Artificial Sequence Synthetic peptide
YTSRLQS

SEQ ID NO: 13 - protein, 9 aa, Artificial Sequence
  Description of Artificial Sequence Synthetic peptide
QQGKTLPYT

SEQ ID NO: 14 - protein, 214 aa, Artificial Sequence
  Description of Artificial Sequence Synthetic polypeptide
DIQMTQSPSSLSASVGDRVTITCRASQDIGNFLNWYQQKPGKAPKLLIYYTSRLQSGVPSRFSGWGSGTDFTLTISSLQPEDFATYYCQQGKTLPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - DNA, 535 bp, Artificial Sequence
  Description of Artificial Sequence Synthetic polynucleotide
cgtgagtagaataactctagaggaatagggaagctaggaagaaactcaaaacatcaagattttaaatacgcttcttggtctccttgctataattatctgggataagcatgctgttttctgtctgtccctaacatgccctgtgattatccgcaaacaacacacccaagggcagaactttgttacttaaacaccatcctgtttgcttctttcctcaggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 16 - protein, 122 aa, Artificial Sequence
  Description of Artificial Sequence Synthetic polypeptide
DVQLVESGGGLVQPGGSRKLSCAASGFTFSNFGMHWVRQAPEKGLEWVAYISSGGSSINYADTVKGRFTISRDNPKNTLFLQMTSLRSEDTAIYYCTRGGTGTRSLYYFDYWGQGATLIVSS

SEQ ID NO: 17 - protein, 452 aa, Artificial Sequence
  Description of Artificial Sequence Synthetic polypeptide
DVQLVESGGGLVQPGGSRKLSCAASGFTFSNFGMHWVRQAPEKGLEWVAYISSGGSSINYADTVKGRFTISRDNPKNTLFLQMTSLRSEDTAIYYCTRGGTGTRSLYYFDYWGQGATLIVSSATTTAPSVYPLVPGCSDTSGSSVTLGCLVKGYFPEPVTVKWNYGALSSGVRTVSSVLQSGFYSLSSLVTVPSSTWPSQTVICNVAHPASKTELIKRIEPRIPKPSTPPGSSCPPGNILGGPSVFIFPPKPKDALMISLTPKVTCVVVDVSEDDPDVHVSWFVDNKEVHTAWTQPREAQYNSTFRVVSALPIQHQDWMRGKEFKCKVNNKALPAPIERTISKPKGRAQTPQVYTIPPPREQMSKKKVSLTCLVTNFFFEAISVEWERNGELEQDYKNTPPILDSDGTYFLYSKLTVDTDSWLQGENFTCSVVHEALHNHHTQKNLSRSPGK

SEQ ID NO: 18 - protein, 107 aa, Artificial Sequence
  Description of Artificial Sequence Synthetic polypeptide
DIQMTQITSSLSVSLGDRVIISCRASQDIGNFLNWYQQKPDGSLKLLIYYTSRLQSGVPSRFSGWGSGTDYSLTISNLEEEDIATFFCQQGKTLPYTFGGGTKLEIK

SEQ ID NO: 19 - protein, 214 aa, Artificial Sequence
  Description of Artificial Sequence Synthetic polypeptide
DIQMTQITSSLSVSLGDRVIISCRASQDIGNFLNWYQQKPDGSLKLLIYYTSRLQSGVPSRFSGWGSGTDYSLTISNLEEEDIATFFCQQGKTLPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 20 - protein, 122 aa, Artificial Sequence
  Description of Artificial Sequence Synthetic polypeptide
QVQLVESGGGVVQPGRSLRLSCAASGFTFSNFGMHWVRQAPGKGLEWVAYISSGGSSINYADTVKGRFTISRDNSKNTLYLQMNSLRAEDTAVYYCTRGGTGTRSLYYFDYWGQGTTVTVSS

SEQ ID NO: 21 - protein, 452 aa, Artificial Sequence
  Description of Artificial Sequence Synthetic polypeptide
QVQLVESGGGVVQPGRSLRLSCAASGFTFSNFGMHWVRQAPGKGLEWVAYISSGGSSINYADTVKGRFTISRDNSKNTLYLQMNSLRAEDTAVYYCTRGGTGTRSLYYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 107 aa, Artificial Sequence
  Description of Artificial Sequence Synthetic polypeptide
DIQMTQSPSSLSASVGDRVTITCRASQDIGNFLNWYQQKPGKAPKLLIYYTSRLQSGVPSRFSGSGSGTDYTLTISSLQPEDFATYYCQQGKTLPYTFGGGTKVEIK

SEQ ID NO: 23 - protein, 214 aa, Artificial Sequence
  Description of Artificial Sequence Synthetic polypeptide
DIQMTQSPSSLSASVGDRVTITCRASQDIGNFLNWYQQKPGKAPKLLIYYTSRLQSGVPSRFSGSGSGTDYTLTISSLQPEDFATYYCQQGKTLPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 122 aa, Artificial Sequence
  Description of Artificial Sequence Synthetic polypeptide
QVQLVESGGGVVQPGRSLRLSCAASGFTFSNFGMHWVRQAPGKGLEWVAYISSGGSSINYADTVKGRFTISRDNSKNTLYLQMNSLRAEDTAVYYCTRGGTGTRSLYYFDYWGQGITVTVSS

SEQ ID NO: 25 - protein, 452 aa, Artificial Sequence
  Description of Artificial Sequence Synthetic polypeptide
QVQLVESGGGVVQPGRSLRLSCAASGFTFSNFGMHWVRQAPGKGLEWVAYISSGGSSINYADTVKGRFTISRDNSKNTLYLQMNSLRAEDTAVYYCTRGGTGTRSLYYFDYWGQGITVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 107 aa, Artificial Sequence
  Description of Artificial Sequence Synthetic polypeptide
DIQMTQSPSSLSASVGDRVTITCRASQDIGNFLNWYQQKPGKAPKLLLYYTSRLQSGVPSRFSGSGSGTDYTLTISSLQPEDFATYYCQQGKTLPYTFGGGTKVEIK

SEQ ID NO: 27 - protein, 214 aa, Artificial Sequence
  Description of Artificial Sequence Synthetic polypeptide
DIQMTQSPSSLSASVGDRVTITCRASQDIGNFLNWYQQKPGKAPKLLLYYTSRLQSGVPSRFSGSGSGTDYTLTISSLQPEDFATYYCQQGKTLPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 119 aa, Artificial Sequence
  Description of Artificial Sequence Synthetic polypeptide
EVTLVESGGDFVKPGGSLKVSCAASGFAFSHYAMSWVRQTPAKRLEWVAYISSGGSGTYYSDSVKGRFTISRDNAKNTLYLQMRSLRSEDSAMYFCTRVKLGTYYFDSWGQGTTLTVSS

SEQ ID NO: 29 - protein, 107 aa, Artificial Sequence
  Description of Artificial Sequence Synthetic polypeptide
DIQMTQTASSLPASLGDRVTISCSASQDISNYLNWYQQKPDGTVKLLIFYSSNLHSGVPSRFSGGGSGTDYSLTISNLEPEDIATYFCHQYSKLPWTFGGGTKLEIK

SEQ ID NO: 30 - protein, 122 aa, Artificial Sequence
  Description of Artificial Sequence Synthetic polypeptide
EVQLVESGGGLVQPGGSLRLSCAASGFTFSNFGMHWVRQAPGKGLEWVAYISSGGSSINYADTVKGRFTISRDNAKNSLYLQMNSLRAEDTAVYYCARGGTGTRSLYYFDYWGQGTLVTVSS

SEQ ID NO: 31 - protein, 107 aa, Artificial Sequence
  Description of Artificial Sequence Synthetic polypeptide
EIVLTQSPATLSLSPGERATLSCRASQDIGNFLNWYQQKPGQAPRLLIYYTSRLQSGIPARFSGSGSGTDFTLTISSLEPEDFAVYYCQQGKTLPYTFGGGTKVEIK

SEQ ID NO: 32 - protein, 122 aa, Artificial Sequence; identical to SEQ ID NO: 30
  Description of Artificial Sequence Synthetic polypeptide

SEQ ID NO: 33 - protein, 107 aa, Artificial Sequence
  Description of Artificial Sequence Synthetic polypeptide
DIQMTQSPSSVSASVGDRVTITCRASQDIGNFLNWYQQKPGKAPKLLIYYTSRLQSGVPSRFSGSGSGTDFTLTISSLQPEDFATYYCQQGKTLPYTFGGGTKVEIK

SEQ ID NO: 34 - protein, 122 aa, Artificial Sequence; identical to SEQ ID NO: 30
  Description of Artificial Sequence Synthetic polypeptide

SEQ ID NO: 35 - protein, 122 aa, Artificial Sequence
  Description of Artificial Sequence Synthetic polypeptide
EVQLVESGGGLVQPGGSLRLSCAASGFTFSNFGMHWVRQAPGKGLEWVAYISSGGSSINYADTVKGRFTISRDNPKNSLYLQMTSLRAEDTAVYYCARGGTGTRSLYYFDYWGQGTLVTVSS

SEQ ID NO: 36 - protein, 107 aa, Artificial Sequence
  Description of Artificial Sequence Synthetic polypeptide
DIQMTQSPSSLSASVGDRVTITCRASQDIGNFLNWYQQKPGKAPKLLIYYTSRLQSGVPSRFSGSGSGTDFTLTISSLQPEDFATYYCQQGKTLPYTFGGGTKVEIK

SEQ ID NO: 37 - protein, 107 aa, Artificial Sequence
  Description of Artificial Sequence Synthetic polypeptide
DIQMTQSPSSLSASVGDRVTITCRASQDIGNFLNWYQQKPDGSLKLLIYYTSRLQSGVPSRFSGSGSGTDFTLTISSLQPEDFATYYCQQGKTLPYTFGGGTKVEIK

SEQ ID NO: 38 - protein, 122 aa, Artificial Sequence
  Description of Artificial Sequence Synthetic polypeptide
EVQLVESGGGLVQPGGSLRLSCAASGFTFSNFGMHWVRQAPEKGLEWVAYISSGGSSINYADTVKGRFTISRDNAKNSLYLQMNSLRAEDTAVYYCARGGTGTRSLYYFDYWGQGTLVTVSS

SEQ ID NO: 39 - protein, 122 aa, Artificial Sequence
  Description of Artificial Sequence Synthetic polypeptide
EVQLVESGGGLVQPGGSLRLSCAASGFTFSNFGMHWVRQAPGKGLEWVAYISSGGSSINYADTVKGRFTISRDNAKNSLYLQMTSLRAEDTAVYYCARGGTGTRSLYYFDYWGQGTLVTVSS

SEQ ID NO: 40 - protein, 122 aa, Artificial Sequence
  Description of Artificial Sequence Synthetic polypeptide
EVQLVESGGGLVQPGGSLRLSCAASGFTFSNFGMHWVRQAPGKGLEWVAYISSGGSSENYADTVKGRFTISRDNAKNSLYLQMNSLRAEDTAVYYCARGGTGTRSLYYFDYWGQGTLVTVSS

SEQ ID NO: 41 - protein, 122 aa, Artificial Sequence
  Description of Artificial Sequence Synthetic polypeptide
EVQLVESGGGLVQPGGSLRLSCAASGFTFSNFGMHWVRQAPGKGLEWVAYISSGGSEIEYADTVKGRFTISRDNAKNSLYLQMNSLRAEDTAVYYCARGGTGTRSLYYFDYWGQGTLVTVSS

SEQ ID NO: 42 - protein, 122 aa, Artificial Sequence
  Description of Artificial Sequence Synthetic polypeptide
EVQLVESGGGLVQPGGSRRLSCAASGFTFSNFGMHWVRQAPGKGLEWVAYISSGGSSINYADTVKGRFTISRDNAKNSLYLQMNSLRAIDTAVYYCARGGTGTRSLYYFDYWGQGTTVIVSS

SEQ ID NO: 43 - protein, 107 aa, Artificial Sequence
  Description of Artificial Sequence Synthetic polypeptide
DIQMTQSPSSLSASVGDRVTITCRASQDIGNFLNWYQQKPGKAPKLLIYYTSRLQSGVPARFSGSGSGTDFTLTISSLQPEDFATYYCQQGKTLPYTFGGGTKVEIK

SEQ ID NO: 44 - protein, 107 aa, Artificial Sequence
  Description of Artificial Sequence Synthetic polypeptide
DIQMTQSPSSLSASVGDRVTITCRASQDIGNFLNWYQQKPGKAPKLLIYYTSRLQSGVPSRFSGSGSGTDFTLTISSLEEEDFATYYCQQGKTLPYTFGGGTKVEIK

SEQ ID NO: 45 - protein, 107 aa, Artificial Sequence
  Description of Artificial Sequence Synthetic polypeptide
DIQMTQSPSSLSASVGDRVTITCRASQDIGNFLNWYQQKPGKAPKLLIYYTSRLQSGVPSRFSGSGSGTDFTLTISSLQPEDFATYFCQQGKTLPYTFGGGTKVEIK

SEQ ID NO: 46 - protein, 107 aa, Artificial Sequence
  Description of Artificial Sequence Synthetic polypeptide
DIQMTQSPSSLSASVGDRVTITCRASQDIGNFLNWYQQKPGKAPKLLIYYTSRLQSGVPSRFSGWGSGTDFTLTISSLQPEDFATYFCQQGKTLPYTFGGGTKVEIK

SEQ ID NO: 47 - protein, 107 aa, Artificial Sequence
  Description of Artificial Sequence Synthetic polypeptide
DIQMTQSPSSLSASVGDRVTITCRASQDIGNFLNWYQQKPGKAPKLLIYYTSRLQSGVPARFSGWGSGTDFTLTISSLQPEDFATYFCQQGKTLPYTFGGGTKVEIK

SEQ ID NO: 48 - protein, 107 aa, Artificial Sequence
  Description of Artificial Sequence Synthetic polypeptide
DIQMTQSPSSLSASVGDRVTITCRASQDIGNFLNWYQQKPGKALKLLIYYTSRLQSGVPARFSGWGSGTDFTLTISSLQPEDFATYFCQQGKTLPYTFGGGTKVEIK

(19) United States
(12) Patent Application Publication
(10) Pub. No.: US 2019/0048073 A1
(43) Pub. Date: Feb. 14, 2019

(54) ANTI-GD3 ANTIBODIES AND ANTIBODY-DRUG CONJUGATES

(71) Applicants: Pfizer Inc., New York, NY (US); Memorial Sloan Kettering Cancer Center, New York, NY (US)

(72) Inventors: Faical Miyara, Bronxville, NY (US); Dhanvanthri S Deevi, Robbinsville, NJ (US); Lioudmila Tchistiakov, Stoneham, MA (US); Michelle Mader, Rensselaer, NY (US); Yijie Gao, Weston, MA (US); Paul Chapman, New York, NY (US); Govind Ragupathi, New York, NY (US)

(21) Appl. No.: 16/039,852

(22) Filed: Jul. 19, 2018

Related U.S. Application Data
(60) Provisional application No. 62/535,120, filed on Jul. 20, 2017.
(60) Provisional application No. 62/697,485, filed on Jul. 13, 2018.

(51) Int. Cl. C07K 16/28; C12N 15/62; A61P 35/00; A61K 47/68; A61K 38/08

(52) U.S. Cl. CPC C07K 16/28; C12N 15/62; A61K 38/08; A61K 47/6803; A61P 35/00

(57) ABSTRACT

The present invention provides for anti-GD3 antibodies, and ADCs and methods for preparing and using the same.

### CROSS REFERENCE TO RELATED APPLICATION

This application claims the benefit of U.S. Provisional Application Nos. 62/535,120, filed Jul. 20, 2017, and 62/697,485, filed Jul. 13, 2018, which are hereby incorporated by reference here in their entireties.

### PARTIES TO A JOINT RESEARCH AGREEMENT

The presently claimed invention was made by or on behalf of the below listed parties to a joint research agreement. The joint research agreement was in effect on or before the date the claimed invention was made and the claimed invention was made as a result of activities undertaken within the scope of the joint research agreement. The parties to the joint research agreement are MEMORIAL SLOAN-KETTERING CANCER CENTER and PFIZER INC.

### SEQUENCE LISTING

The instant application contains a Sequence Listing which has been submitted electronically in ASCII format and is hereby incorporated by reference in its entirety. Said ASCII copy, created on Jul. 19, 2018, is named PCFC-0078-101-SL.txt and is 61,751 bytes in size.

### FIELD OF THE INVENTION

The present invention relates to ganglioside GD3 (GD3) antibodies and antibody-drug conjugates (ADCs). The present invention further relates to the methods of using such antibodies and ADCs for the treatment of cancer.

### BACKGROUND

Glycosphingolipids contribute to the glycoprotein-polysaccharide (glycocalyx) covering that surrounds all eukaryotic cells (along with other glycoproteins and glycosaminoglycans). Glycosphingolipids are lipids that contain a sphingoid base and one or more sugar residues. A ganglioside, such as GD3, is comprised of a glycosphingolipid (ceramide and oligosaccharide) with one or more sialic acids present on the sugar chain (Kolter, 2012, ISRN Biochem:506160). GD3 is defined by the chemical structure: Neu5Acα2,8NeuAcα2,3Galβ1,4Glcβ1Cer (Haji-Ghassemi et al., 2015, 25(9):920-952). These chemical structures are evolutionarily conserved across species (Irvine & Seyfried, 1994, Comp Biochem Physiol B Biochem Mol Biol 109(4):603-612; Variki, 2011, Cold Spring Harb Perspect Biol 3(6):a005462).

GD3 is found in multiple tissues across species including mouse, rat, dog, monkey, human and other mammals (Helfand et al., 1999, Cancer Res 59(13):3119-3127; Kasahara et al., 1997, J Biol Chem 272(47):29947-29953). Cell surface GD3, along with other gangliosides, is expressed on cells of the neural crest lineage during embryogenesis of vertebrates and eventually undergoes profound changes in the levels of expression throughout development (Kasahara et al, 1997). GD3 is highly expressed during early developmental stages within the central nervous system when neuronal cells actively proliferate (Popa et al., 2007, Glycobiology 17(4):367-373; Nagai & Iwamori, 1995, Biology of the sialic acids, 197-241). At later developmental stages, GD3 expression declines and other gangliosides become the major species displayed on cells (Seyfried & Yu, 1985, Mol Cell Biochem 68:3-10). GD3 is expressed at low levels on normal adult tissues, including melanocytes, adrenal medulla, islet cells of the pancreas, astrocytes, and subpopulations of keratinocytes and T lymphocytes (Graus et al., 1984, Brain Research 324:190-194; Real et al., 1985, Cancer Research 45:4401; Garin-Chesa et al., 1989, American Journal of Pathology 134:2).

In contrast to normal adult tissues, GD3 is highly expressed on certain tumor cells (Hakomori & Kannagi, 1983, Natl Cancer Inst 71(2):231-251; Portoukalian et al., 1991, Int J Cancer 2:49(6):893-899) and its increased expression may contribute to tumorigenesis through effects on cell migration, adhesion, proliferation and differentiation (Daniotti et al., 2002, Neurochem Res 27(11):1421-1429; Birkle et al., 2003, Biochimie 85:455-463). GD3 expression was reported in 58 out of 61 human melanoma tumors, including 7 out of 8 metastatic lesions to the liver (Real et al., 1985, Cancer Research 45:4401). Human melanoma cells from primary tumors express elevated levels of GD3 irrespective of their BRAF mutational status (Tringali et al., 2014, BMC Cancer 14:560). GD3 is also overexpressed in neuroectodermal tumors (e.g., neuroblastoma and glioma) (Campanella, 1992, J Neurosurg Sci 36(1):11-25; Hedberg et al., 2000, Glycoconj J 17(10):717-726; Hedberg et al., 2001, Neuropathol Appl Neurobiol 27(6):451-64), soft tissue sarcomas (Chang et al., 1992, Cancer 70(3):633-638) and carcinomas, including small cell lung (Spitalnik et al., 1986, Cancer Res 46(9):4751-4755; Brezicka et al., 2000, Lung Cancer 28(1):29-36), breast (Marquina et al., 1996, Cancer Res 56(22):5165-5171), colon, pancreas (Fredman et al., 1983, 61(1):45-48), prostate (Fabbri et al., 2011, J Cell Physiol 226(11):3035-3042), and ovary (Lo et al., 2010, Clin Cancer Res 16(10):2769-2680). In addition, GD3 expression was shown to be present on T-cell acute lymphoblastic leukemia and absent from other non-T cell lymphocyte malignancies (Reaman et al., 1990, Cancer Res 50(1):202-205).

There remains a significant need for additional therapeutic options for cancers. To this end, the present invention provides novel antibodies and ADCs that target GD3 expressing cancers.

### SUMMARY OF THE INVENTION

The invention provides antibodies (and antigen-binding fragments thereof) and antibody-drug-conjugates that specifically bind to GD3, as well as uses, and associated methods thereof. Those skilled in the art will recognize, or be able to ascertain using no more than routine experimentation, many equivalents to the specific embodiments of the invention described herein. Such equivalents are intended to be encompassed by the following embodiments (E).

E1. An antibody or antigen-binding fragment thereof, that specifically binds to GD3.

E2. The antibody, or antigen-binding fragment thereof, of E1, comprising the heavy chain variable region complementarity determining region 1 (CDR-H1), CDR-H2, and CDR-H3 sequences of SEQ ID NO: 1.

E3. The antibody, or antigen-binding fragment thereof, of E1 or E2, comprising a heavy chain variable region (VH) that comprises:
(a) a VH CDR-H1 comprising the amino acid sequence of SEQ ID NO: 2,
(b) a VH CDR-H2 comprising the amino acid sequence of SEQ ID NO: 4, and
(c) a VH CDR-H3 comprising the amino acid sequence of SEQ ID NO: 6.

E4. The antibody, or antigen-binding fragment thereof, of E1-E3, comprising a heavy chain variable region (VH) that comprises:
(a) a VH CDR-H1 comprising the amino acid sequence of SEQ ID NO: 3,
(b) a VH CDR-H2 comprising the amino acid sequence of SEQ ID NO: 5, and
(c) a VH CDR-H3 comprising the amino acid sequence of SEQ ID NO: 6.

E5. The antibody, or antigen-binding fragment thereof, of any one of E1-E4, comprising a human VH germline consensus framework sequence.

E6. The antibody, or antigen-binding fragment thereof, of any one of E1-E5, comprising a VH framework sequence derived from a human germline VH sequence selected from the group consisting of: DP54, DP-50, IGHV3-30*09, IGHV3-30*15, IGHV3-48*01, DP-77, DP-51, IGHV3-66*01, DP-53, DP-48, IGHV3-53*01, IGHV3-30*02, and DP-49.

E7. The antibody, or antigen-binding fragment thereof, of any one of E1-E6, wherein the VH framework sequence is at least 91%, at least 92%, at least 93%, at least 94%, at least 95%, at least 96%, at least 97%, at least 98%, at least 99%, or 100% identical to the human germline framework sequence from which it is derived.

E8. The antibody, or antigen-binding fragment thereof, of any one of E1-E7, comprising a VH framework sequence derived from a human germline DP54 sequence.

E9. The antibody, or antigen-binding fragment thereof, of any one of E1-E8, comprising a VH framework sequence wherein the residue at position 74 of the VH domain, according to the numbering of SEQ ID NO: 1, is a proline amino acid residue.

E10. The antibody, or antigen-binding fragment thereof, of any one of embodiments E1-E9, comprising a VH comprising an amino acid sequence at least 90% identical to SEQ ID NO: 1.

E11. The antibody, or antigen-binding fragment thereof, of any one of embodiments E1-E10, comprising a VH comprising an amino acid sequence at least 90% identical to SEQ ID NO: 1, wherein, according to the numbering of SEQ ID NO: 1, the amino acid residue at position 1 is glutamic acid, the amino acid residue at position 11 is leucine, the amino acid residue at position 16 is glycine, the amino acid residue at position 74 is proline, the amino acid residue at position 77 is serine, the amino acid residue at position 93 is alanine, and the amino acid residue at position 108 is leucine.

E12. The antibody, or antigen-binding fragment thereof, of any one of embodiments E1-E11, comprising a VH whose framework sequence is at least 92%, 93%, 94%, 95%, 96%, 97%, 98%, or 99% identical to the framework sequence of SEQ ID NO: 1.

E13. The antibody, or antigen-binding fragment thereof, of any one of embodiments E1-E12, comprising a VH whose framework sequence is at least 92%, 93%, 94%, 95%, 96%, 97%, 98%, or 99% identical to the framework sequence of SEQ ID NO: 1, and wherein, according to the numbering of SEQ ID NO: 1, the amino acid residue at position 1 is glutamic acid, the amino acid residue at position 11 is leucine, the amino acid residue at position 16 is glycine, the amino acid residue at position 74 is proline, the amino acid residue at position 77 is serine, the amino acid residue at position 93 is alanine, and the amino acid residue at position 108 is leucine.

E14. The antibody, or antigen-binding fragment thereof, of any one of embodiments E1-E13, comprising a VH comprising the amino acid sequence of SEQ ID NO: 1.

E15. The antibody, or antigen-binding fragment thereof, of any one of E1-E14, comprising the CDR-L1, CDR-L2, and CDR-L3 sequences of SEQ ID NO: 9.

E16. The antibody, or antigen-binding fragment thereof, of any one of E1-E15, comprising a light chain variable region (VL) that comprises:
(a) a VL complementarity determining region one (CDR-L1) comprising the amino acid sequence of SEQ ID NO: 10,
(b) a VL CDR-L2 comprising the amino acid sequence of SEQ ID NO: 12, and
(c) a VL CDR-L3 comprising the amino acid sequence of SEQ ID NO: 13.

E17. The antibody, or antigen-binding fragment thereof, of any one of E1-E16, comprising a light chain variable region (VL) that comprises:
(a) a VL CDR-L1 comprising the amino acid sequence of SEQ ID NO: 11,
(b) a VL CDR-L2 comprising the amino acid sequence of SEQ ID NO: 12, and
(c) a VL CDR-L3 comprising the amino acid sequence of SEQ ID NO: 13.

E18. The antibody, or antigen-binding fragment thereof, of any one of E1-E17, comprising a human VL germline consensus framework sequence.

E19. The antibody, or antigen-binding fragment thereof, of any one of E1-E18, wherein the VL framework sequence is at least 98%, at least 99%, or 100% identical to the human germline framework sequence from which it is derived.

E20. The antibody, or antigen-binding fragment thereof, of any one of E1-E19, comprising a VL framework sequence selected from the group consisting of DPK9, DPK5, DPK4, DPK1, IGKV1-5*01, DPK24, DPK21, DPK15, IGKV1-13*02, IGKV1-17*01, DPK8, IGKV3-11*01, and DPK22.

E21. The antibody, or antigen-binding fragment thereof, of any one of E1-E20, comprising a VL framework sequence selected from the group consisting of DPK9, DPK5, DPK4, DPK1, and IGKV1-5*01.

E22. The antibody, or antigen-binding fragment thereof, of any one of E1-E21, comprising a VL framework sequence derived from a human germline DPK9 sequence.

E23. The antibody, or antigen-binding fragment thereof, of any one of E1-E22, comprising a VL framework sequence wherein the residue at position 65 of the VL domain, according to the numbering of SEQ ID NO: 9, is a tryptophan amino acid residue.

E24. The antibody, or antigen-binding fragment thereof, of any one of E1-E23, comprising a VL comprising an amino acid sequence at least 90% identical to SEQ ID NO: 9.

E25. The antibody, or antigen-binding fragment thereof, of any one of embodiments E1-E24, comprising a VL comprising an amino acid sequence at least 90% identical to SEQ ID NO: 9, wherein, according to the numbering of SEQ ID NO: 9, the amino acid residue at position 65 is tryptophan, and the amino acid residue at position 71 is phenylalanine.

E26. The antibody, or antigen-binding fragment thereof, of any one of embodiments E1-E25, comprising a VL whose framework sequence is at least 66%, 74%, 76%, 80%, 90%, 91%, 92%,

E27. The antibody, or antigen-binding fragment thereof, of any one of embodiments E1-E26, comprising a VH whose framework sequence is at least 66%, 74%, 76%, 80%, 90%, 91%, 92%, 93%, 94%, 95%, 96%, 97%, 98%, or 99% identical to the framework sequence of SEQ ID NO:9, and wherein, according to the numbering of SEQ ID NO: 9, the amino acid residue at position 65 is tryptophan, and the amino acid residue at position 71 is phenylalanine.

93%, 94%, 95%, 96%, 97%, 98%, or 99% identical to the framework sequence of SEQ ID NO:9.

E28. The antibody, or antigen-binding fragment thereof, of any one of embodiments E1-E27, comprising a VL whose framework sequence is at least 96%, 97%, 98%, or 99% identical to the framework sequence of SEQ ID NO:9.

E29. The antibody, or antigen-binding fragment thereof, of any one of embodiments E1-E28, comprising a VL comprising the amino acid sequence of SEQ ID NO: 9.

E30. An isolated antibody, or antigen-binding fragment thereof, that specifically binds GD3, comprising the CDR-H1, CDR-H2, and CDR-H3 sequences of SEQ ID NO: 1, and the CDR-L1, CDR-L2, and CDR-L3 sequences of SEQ ID NO: 9.

E31. An isolated antibody, or antigen-binding fragment thereof, that specifically binds GD3 comprising:
(i) a VH that comprises:
   (a) a CDR-H1 comprising the amino acid sequence of SEQ ID NO: 2,
   (b) a CDR-H2 comprising the amino acid sequence of SEQ ID NO: 4, and
   (c) a CDR-H3 comprising the amino acid sequence of SEQ ID NO: 6;
and (ii) a VL that comprises:
   (a) a CDR-L1 comprising the amino acid sequence of SEQ ID NO: 10,
   (b) a CDR-L2 comprising the amino acid sequence of SEQ ID NO: 12, and
   (c) a CDR-L3 comprising the amino acid sequence of SEQ ID NO: 13.

E32. An isolated antibody, or antigen-binding fragment thereof, that specifically binds GD3 comprising:
(i) a VH that comprises:
   (a) a CDR-H1 comprising the amino acid sequence of SEQ ID NO: 3,
   (b) a CDR-H2 comprising the amino acid sequence of SEQ ID NO: 5, and
   (c) a CDR-H3 comprising the amino acid sequence of SEQ ID NO: 6;
and (ii) a VL that comprises:
   (a) a CDR-L1 comprising the amino acid sequence of SEQ ID NO: 11,
   (b) a CDR-L2 comprising the amino acid sequence of SEQ ID NO: 12, and
   (c) a CDR-L3 comprising the amino acid sequence of SEQ ID NO: 13.

E33. The antibody, or antigen-binding fragment thereof, of any one of E1-E32, comprising an Fc domain.

E34. The antibody, or antigen-binding fragment thereof, of E33, wherein the Fc domain is the Fc domain of an IgA, IgD, IgE, IgM, or IgG.

E35. The antibody, or antigen-binding fragment thereof, of E34 wherein the Fc domain is the Fc domain of an IgG.

E36. The antibody, or antigen-binding fragment thereof, of E35, wherein the IgG is selected from the group consisting of IgG₁, IgG₂, IgG₃, or IgG₄.

E37. The antibody, or antigen-binding fragment thereof, of E36, wherein the IgG is IgG₁.

E38. The antibody, or antigen-binding fragment thereof, of any one of embodiments E33-E37, comprising a HC comprising the amino acid sequence of SEQ ID NO: 1.

E39. The antibody, or antigen-binding fragment thereof, of any one of embodiments E33-E38, comprising a LC comprising the amino acid sequence of SEQ ID NO: 9.

E40. The antibody, or antigen-binding fragment thereof, of any one of E1-E39, comprising the VH amino acid sequence encoded by the plasmid deposited at the ATCC and having ATCC Accession No. PTA-124057.

E41. The antibody, or antigen-binding fragment thereof, of any one of E1-E39, comprising the VL amino acid sequence encoded by the plasmid deposited at the ATCC and having ATCC Accession No. PTA-124058.

E42. The antibody, or antigen-binding fragment thereof, of any one of E1-E41, wherein the antibody or antigen-binding fragment is an Fc fusion protein, a monobody, a maxibody, a bifunctional antibody, an scFab, an scFv, or a peptibody.

E43. The antibody, or antigen-binding fragment thereof, of any one of E1-E42, wherein the antibody or antigen-binding fragment has a similarity score of approximately 0.8 with a lysosomal marker.

E44. An antibody, or antigen-binding fragment thereof, that competes for binding to GD3 with an antibody or antigen-binding fragment thereof of any one of E1-E43.

E45. An isolated nucleic acid molecule encoding the antibody, or antigen-binding fragment thereof, of any one of E1-E44.

E46. An isolated nucleic acid molecule comprising the nucleic acid sequence as set forth as SEQ ID NO: 8 or at least 95% identical thereto.

E47. An isolated nucleic acid molecule comprising the nucleic acid sequence as set forth as SEQ ID NO: 15 or at least 95% identical thereto.

E48. An isolated nucleic acid molecule comprising the coding sequence of the nucleic acid insert of the plasmid deposited with the ATCC and having Accession No. PTA-124057.

E49. An isolated nucleic acid molecule comprising the coding sequence of the nucleic acid insert of the plasmid deposited with the ATCC and having Accession No. PTA-124058.

E50. A vector comprising the nucleic acid molecule of any one of E45-E49.

E51. A host cell comprising the nucleic acid molecule of any one of E45-E50, or the vector of E50.

E52. The host cell of E51, wherein said cell is a mammalian cell.

E53. The host cell of E52, wherein said host cell is a CHO cell, a HEK-293 cell, or a Sp2.0 cell.

E54. A method of making an antibody or antigen-binding fragment thereof, comprising culturing the host cell of E51-E53 under a condition wherein said antibody or antigen-binding fragment is expressed by said host cell.

E55. The method of E54, further comprising isolating said antibody or antigen-binding fragment thereof.

E56. The antibody, or antigen-binding fragment thereof, of any one of E1-E44, wherein the terminal plasma half-life in mice is at least one or more of about 1 day, about 1.5 days, about 2 days, about 2.5 days, about 3 days, about 3.5 days, about 4 days, about 4.5 days, about 5 days, about 5.5 days, about 6 days, about 6.5 days, about 7 days, about 7.5 days, about 8 days, about 8.5 days, about 9 days, about 9.5 days, about 10 days, about 10.5 days and about 10.9 days.

E57. The antibody, or antigen-binding fragment thereof, of any one of E1-E44 and E56, wherein the terminal plasma half-life in mice is at least 10.6 days or 10.9 days.

E58. The antibody, or antigen-binding fragment thereof, of any one of E1-E44 and E56-E57, wherein the terminal plasma half-life in rats is at least one or more of about 1 day, about 1.5 days, about 2 days, about 2.5 days, about 3 days, about 3.5 days, about 4 days, about 4.5 days, about 5 days, about 5.5 days, about 6 days, about 6.5 days, about 7 days, about 7.5 days, about 8 days, about 8.5 days, about 9 days, about 9.5 days, about 10 days, about 10.5 days, about 11 days, about 11.5 days, about 12 days, about 12.5 days, about 13 days, about 13.5 days and about 13.7 days.

E59. The antibody, or antigen-binding fragment thereof, of any one of E1-E44 and E56-E58, wherein the terminal plasma half-life in rats is at least 12.3 days or 13.7 days.

E60. The antibody, or antigen-binding fragment thereof, of any one of E1-E44 and E56-E59, wherein the terminal plasma half-life in cynomolgus monkeys is at least one or more of about 1 day, about 1.5 days, about 2 days, about 2.5 days, about 3 days, about 3.5 days, about 4 days, about 4.5 days, about 5 days, about 5.5 days, about 6 days, about 6.5 days, about 7 days, about 7.5 days, about 8 days, about 8.5 days, about 9 days, about 9.5 days, about 10 days, about 10.5 days, about 11 days, about 11.5 days, about 12 days, about 12.5 days, about 13 days, about 13.5 days, about 14 days, about 14.5 days, about 15 days, about 15.5 days, and about 16 days.

E61. The antibody, or antigen-binding fragment thereof, of any one of E1-E44 and E56-E60, wherein the terminal plasma half-life in cynomolgus monkeys is at least 10.8 days, 13 days or 16 days.

E62. The antibody, or antigen-binding fragment thereof, of any one of E1-E44 and E56-E61, wherein the terminal plasma half-life in humans is at least one or more of about 1 day, about 1.5 days, about 2 days, about 2.5 days, about 3 days, about 3.5 days, about 4 days, about 4.5 days, about 5 days, about 5.5 days, about 6 days, about 6.5 days, and about 7 days.

E63. The antibody, or antigen-binding fragment thereof, of any one of E1-E44 and E56-E62, wherein the terminal plasma half-life in humans is at least 7 days.

E64. A pharmaceutical composition comprising an antibody or antigen-binding fragment thereof of any one of E1-E63, and a pharmaceutically acceptable carrier or excipient.

E65. A method of treating a disease or disorder associated with GD3 cell surface expression or a disorder associated with elevated levels of GD3 activity, comprising administering to a subject in need thereof a therapeutically effective amount of the antibody, or antigen-binding fragment thereof, of any one of embodiments E1-E63, or the pharmaceutical composition of E64.

E66. The method of E65, comprising administering to a subject in need thereof 0.5 mg/kg of the antibody, or antigen-binding fragment thereof, of any one of embodiments E1-E63, or the pharmaceutical composition of E64.

E67. The method of E65 or E66, wherein said disease or disorder is melanoma, breast cancer, glioma, glioblastoma, or lung cancer.

E68. The method of any one of E65-E67, comprising administering said antibody or antigen-binding fragment thereof, or pharmaceutical composition, intravenously.

E69. The method of any one of E65-E68, wherein said antibody or antigen-binding fragment thereof, or pharmaceutical composition, is administered about twice a week, once a week, once every two weeks, once every three weeks, once every four weeks, once every five weeks, once every six weeks, once every seven weeks, once every eight weeks, once every nine weeks, once every ten weeks, twice a month, once a month, once every two months, once every three months, or once every four months.

E70. The antibody, or antigen-binding fragment thereof, of any one of E1-E63, or the pharmaceutical composition of E64, for use as a medicament.

E71. An antibody-drug conjugate (ADC) of the formula, Ab-(L-D)p, wherein:
Ab is an antibody, or antigen-binding fragment thereof, that specifically binds GD3;
L-D is a linker-drug moiety, wherein L is a linker, and D is a drug;
p is an integer from about 1 to 12.

E72. The ADC of E71, wherein p is 1.

E73. The ADC of E71, wherein p is 2.

E74. The ADC of E71, wherein p is 3.

E75. The ADC of E71, wherein p is 4.

E76. The ADC of E71, wherein p is 5.

E77. The ADC of E71, wherein p is 6.

E78. The ADC of E71, wherein p is 7.

E79. The ADC of E71, wherein p is 8.

E80. The ADC of E71, wherein p is 9.

E81. The ADC of E71, wherein p is 10.

E82. The ADC of E71, wherein p is 11.

E83. The ADC of E71, wherein p is 12.

E84. The ADC of any one of E71-E83, wherein the antibody, or antigen-binding fragment thereof is the antibody, or antigen-binding fragment thereof, of any one of E1-E52 or E70.

E85. The ADC of any one of E71-E84, wherein the linker is stable or hydrolysable.

E86. The ADC of any one of E71-E85, wherein the linker comprises a hydrazone-, disulfide- or a peptide-based linker.

E87. The ADC of any one of E71-E86, wherein the linker comprises a linker having the formula, (CO-Alk¹-Sp¹-Ar-Sp²-Alk²-C(Z¹)=Q-Sp), wherein:
(a) Alk¹ and Alk² are independently a bond or branched or unbranched (C₁-C₁₀) alkylene chain;
(b) Sp¹ is a bond, —S—, —O—, —CONH—, —NHCO—, —NR′—, —N(CH₂CH₂)₂N—, or —X—Ar′—Y—(CH₂)ₙ—Z wherein X, Y, and Z are independently a bond, —NR′—, —S—, or —O—, with the proviso that when n=0, then at least one of Y and Z must be a bond and Ar′ is 1,2-, 1,3-, or 1,4-phenylene optionally substituted with one, two, or three groups of (C₁-C₅) alkyl, (C₁-C₄) alkoxy, (C₁-C₄) thioalkoxy, halogen, nitro, —COOR′, —CONHR′, —(CH₂)ₙCOOR′, —S(CH₂)ₙCOOR′, —O(CH₂)ₙCONHR′, or —S(CH₂)ₙCONHR′, with the proviso that when Alk′ is a bond, Sp¹ is a bond; n is an integer from 0 to 5; R′ is a branched or unbranched (C₁-C₅) chain optionally substituted by one or two groups of —OH, (C₁-C₄) alkoxy, (C₁-C₄) thioalkoxy, halogen, nitro, (C₁-C₃) dialkylamino, or (C₁-C₃) trialkylammonium -A⁻ where A⁻ is a pharmaceutically acceptable anion completing a salt; (c) Ar is 1,2-, 1,3-, or 1,4-phenylene optionally substituted with one, two, or three groups of (C₁-C₆) alkyl, (C₁-C₅) alkoxy, (C₁-C₄) thioalkoxy, halogen, nitro, —COOR′, —CONHR′, —O(CH₂)ₙCOOR′, —S(CH₂)ₙCOOR′, —O(CH₂)ₙCONHR′, or —S(CH₂)ₙCONHR′ wherein n and R′ are as hereinbefore defined or a 1,2-, 1,3-, 1,4-, 1,5-, 1,6-, 1,7-, 1,8-, 2,3-, 2,6-, or 2,7-naphthylidene or

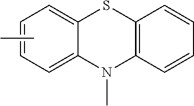

with each naphthylidene or phenothiazine optionally substituted with one, two, three, or four groups of (C₁-C₆) alkyl, (C₁-C₅) alkoxy, (C₁-C₄) thioalkoxy, halogen, nitro, —COOR′, —CONHR′, —O(CH₂)ₙCOOR′, —S(CH₂)ₙCOOR′, or —S(CH₂)ₙCONHR′ wherein n and R′ are as defined above, with the proviso that when Ar is phenothiazine, Sp¹ is a bond only connected to nitrogen;
(d) Sp² is a bond, —S—, or —O—, with the proviso that when Alk² is a bond, Sp² is a bond,
(e) Z¹ is H, (C₁-C₅) alkyl, or phenyl optionally substituted with one, two, or three groups of (C₁-C₅) alkyl, (C₁-C₅) alkoxy, (C₁-C₄) thioalkoxy, halogen, nitro, —COOR′, —ONHR′, —O(CH₂)ₙCOOR′, —S(CH₂)ₙCOOR′, —O(CH₂)ₙCONHR′, or —S(CH₂)ₙCONHR′ wherein n and R′ are as defined above;
(f) Sp is a straight or branched-chain divalent or trivalent (C₁-C₁₈) radical, divalent or trivalent aryl or heteroaryl radical, divalent or trivalent (C₃-C₁₈) cycloalkyl or heterocycloalkyl radical, divalent or trivalent aryl- or heteroaryl-aryl (C₁-C₁₈) radical, divalent or trivalent cycloalkyl- or heterocycloalkyl-alkyl (C₁-C₁₈) radical or divalent or trivalent (C₂-C₁₈) unsaturated alkyl radical, wherein heteroaryl is preferably furyl, thienyl, N-methylpyrrolyl, pyridinyl, N-methylimidazolyl, oxazolyl, pyrimidinyl, quinolyl, isoquinolyl, N-methylcarbazoyl, aminocourmarinyl, or phenazinyl and wherein if Sp is a trivalent radical, Sp may be additionally substituted by lower (C₁-C₅) dialkylamino, lower (C₁-C₅) alkoxy, hydroxy, or lower (C₁-C₅) alkylthio groups; and
(g) Q is ═NHNCO—, ═NHNCS—, ═NHNCONH—, ═NHNCSNH—, or ═NHO—.

E88. The ADC of any one of E71-E87, wherein:
(a) Alk¹ is a branched or unbranched (C₁-C₁₀) alkylene chain; Sp′ is a bond, —S—, —O—, —CONH—, —NHCO—, or —NR′ wherein R′ is as hereinbefore defined, with the proviso that when Alk′ is a bond, Sp¹ is a bond;
(b) Ar is 1,2-, 1,3-, or 1,4-phenylene optionally substituted with one, two, or three groups of (C₁-C₆) alkyl, (C₁-C₅) alkoxy, (C₁-C₄) thioalkoxy, halogen, nitro, —COOR′, —CONHR′, —O(CH₂)ₙCOOR′, —S(CH₂)ₙCOOR′, —O(CH₂)ₙCONHR′, or —S(CH₂)ₙCONHR′ wherein n and R′ are as hereinbefore defined, or Ar is a 1,2-, 1,3-, 1,4-, 1,5-, 1,6-, 1,7-, 1,8-, 2,3-, 2,6-, or 2,7-naphthylidene each optionally substituted with one, two, three, or four groups of (C₁-C₆) alkyl, (C₁-C₅) alkoxy, (C₁-C₄) thioalkoxy, halogen, nitro, —COOR′, —CONHR′, —O(CH₂)ₙCOOR′, —S(CH₂)ₙCOOR′, —O(CH₂)ₙCONHR′, or —S(CH₂)ₙCONHR′.
(c) Z¹ is (C₁-C₅) alkyl, or phenyl optionally substituted with one, two, or three groups of (C₁-C₅) alkyl, (C₁-C₄) alkoxy, (C₁-C₄) thioalkoxy, halogen, nitro, —COOR′, —CONHR′, —O(CH₂)ₙCOOR′, —S(CH₂)ₙCOOR′, —O(CH₂)ₙCONHR′, or —S(CH₂)ₙCONHR′; and
(d) Alk² and Sp² are together a bond.

E89. The ADC of any one of E71-E88, wherein the linker comprises a maleimidocapronic-valine-citruline-p-aminobenzyloxycarbonyl linker (mcValCitPABC), 4-(4-acetylphenoxy) butanoic acid, (3-Acetylphenyl) acetic acid, 4-mercapto-4-methyl-pentanoic acid, valine-citrulline, a phenylalanine-lysine linker Sulfosuccinimidyl-4-[N-maleimidomethyl]cyclohexane-1-carboxylate, maleimidocaproyl, diethylenetriamine pentaacetate-isothiocyanate, succinimidyl 6-hydrazinium nicotinate hydrochloride, or hexamethylpropylene amine oxime.

E90. The ADC of any one of E71-E89, wherein the linker comprises mcValCitPABC.

E91. The ADC of any one of E71-E90, where in the drug comprises a therapeutic agent, a detectable label, or a binding agent.

E92. The ADC of any one of E71-E91, where in the drug exerts a cytotoxic, cytostatic, and/or immunomodulatory effect on cancer cells or activated immune cells.

E93. The ADC of any one of E71-E92, wherein the drug is selected from the group consisting of cytotoxic agent, chemotherapeutic agent, cytostatic agent, an anti-angiogenic agent, an anti-proliferative agent, a pro-apoptotic agent, and an immunomodulatory agent.

E94. The ADC of any one of E71-E93, wherein the drug is a drug selected from the group consisting of anthracycline, an auristatin, CC-1065, a dolastatin, a duocarmycin, an enediyne, a geldanamycin, a maytansine, a puromycin, a taxane, a vinca alkaloid, SN-38, tubulysin, hemiasterlin, and stereoisomers, isosteres, analogs or derivatives thereof.

E95. The ADC of any one of E71-E94, wherein the auristatin is selected from the group consisting of auristatin 0101, auristatin D, auristatin E, auristatin EB, auristatin EFP, monomethyl auristatin D, monomethyl auristatin F, and 5-benzoylvaleric acid-auristatin E.

E96. The ADC of any one of E71-E95, wherein the drug is auristatin 0101.

E97. The ADC of any one of E71-E96, wherein the linker is mcValCitPABC and the drug is auristatin 0101.

E98. The ADC of any one of E71-E97, wherein the antibody, or antigen-binding fragment thereof is the antibody, or antigen-binding fragment thereof, of any one of E1-E47, the linker is mcValCitPABC, and the drug is auristatin 0101.

E99. The ADC of any one of E71-E98, wherein the antibody, or antigen-binding fragment thereof comprises a VH comprising the amino acid sequence of SEQ ID NO: 1 and a VL comprising the amino acid sequence of SEQ ID NO: 9, the linker is mcValCitPABC, and the drug is auristatin 0101.

E100. The ADC of any one of E71-E99, wherein the antibody, or antigen-binding fragment thereof comprises a heavy chain (HC) comprising the amino acid sequence of SEQ ID NO: 7 and a light chain (LC) comprising the amino acid sequence of SEQ ID NO: 14, the linker is mcValCitPABC, and the drug is auristatin 0101.

E101. The ADC of any one of E71-E100, wherein the ADC has a similarity score of about 0.9 to 1.1 with a lysosomal marker.

E102. The ADC of any one of E71-E101, wherein the average tumor volume in a mouse SK-MEL-19 metastatic melanoma xenograft model wherein the ADC is administered to the mouse at 10 mg/kg of body weight, every 4^{th} day for 16 days. is less than about 196 mm³ at day 1, about 234 mm³ at day 5, about 207 mm³ at day 8, about 249 mm³ at day 12, about 337 mm³ at day 15, about 337 mm³ at day 19, about 333 mm³ at day 22, about 359 mm³ at day 26, about 374 mm³ at day 29, or about 366 mm³ at day 33.

E103. The ADC of any one of E71-E102, wherein the average tumor volume in a mouse SK-MEL-19 metastatic melanoma xenograft model wherein the ADC is administered to the mouse at 10 mg/kg of body weight, every 4^{th} day for 16 days. is less than about 196 mm³ at day 1, about 234 mm³ at day 5, about 207 mm³ at day 8, about 249 mm³ at day 12, about 337 mm³ at day 15, about 337 mm³ at day 19, about 333 mm³ at day 22, about 359 mm³ at day 26, about 374 mm³ at day 29, or about 366 mm³ at day 33, and further wherein the average tumor volume in the mouse model where the ADC is not administered is less than about 599 mm³ at day 1, about 642 mm³ at day 5, about 693 mm³ at day 8, about 654 mm³ at day 12, about 663 mm³ at day 15, about 689 mm³ at day 19, about 838 mm³ at day 22, about 869 mm³ at day 26, about 969 mm³ at day 29, or about 1,126 mm³ at day 33.

E104. The ADC of any one of E71-E103, wherein the average tumor volume in a mouse SK-MEL-19 metastatic melanoma xenograft model wherein the ADC is administered at 10 mg/kg of body weight, every 4^{th} day for 16 days, is about 144 mm³ to about 196 mm³ at day 1, about 176 mm³ to about 234 mm³ at day 5, about 139 mm³ to about 207 mm³ at day 8, about 165 mm³ to about 249 mm³ at day 12, about 235 mm³ to about 337 mm³ at day 15, about 235 mm³ to about 337 mm³ at day 19, about 211 mm³ to about 333 mm³ at day 22, about 191 mm³ to about 359 mm³ at day 26, about 200 mm³ to about 374 mm³ at day 29, or about 190 mm³ to about 366 mm³ at day 33.

E105. The ADC of any one of E71-E104, wherein the average tumor volume in a mouse SK-MEL-19 metastatic melanoma xenograft model wherein the ADC is administered at 10 mg/kg of body weight, every 4^{th} day for 16 days, is about 144 mm³ to about 196 mm³ at day 1, about 176 mm³ to about 234 mm³ at day 5, about 139 mm³ to about 207 mm³ at day 8, about 165 mm³ to about 249 mm³ at day 12, about 235 mm³ to about 337 mm³ at day 15, about 235 mm³ to about 337 mm³ at day 19, about 211 mm³ to about 333 mm³ at day 22, about 191 mm³ to about 359 mm³ at day 26, about 200 mm³ to about 374 mm³ at day 29, or about 190 mm³ to about 366 mm³ at day 33, and further wherein the average tumor volume in an otherwise identical mouse wherein the ADC is not administered is about 363 mm³ to about 599 mm³ at day 1, about 410 mm³ to about 642 mm³ at day 5, about 465 mm³ to about 693 mm³ at day 8, about 444 mm³ to about 654 mm³ at day 12, about 437 mm³ to about 663 mm³ at day 15, about 463 mm³ to about 689 mm³ at day 19, about 608 mm³ to about 838 mm³ at day 22, about 637 mm³ to about 869 mm³ at day 26, about 753 mm³ to about 969 mm³ at day 29, or about 838 mm³ to about 1,126 mm³ at day 33.

E106. The ADC of any one of E73-E105, wherein the average tumor volume in a mouse SK-MEL-19 metastatic melanoma xenograft model is 190 mm³ to 366 mm³ at day 33 after administration of the antibody-drug conjugate at 10 mg/kg of body weight, every 4^{th} day for 16 days.

E107. The ADC of any one of E71-E106, wherein the average tumor volume in a mouse SK-129862F(PDX) metastatic melanoma xenograft model wherein the ADC is administered at 10 mg/kg of body weight, every 4^{th} day for 16 days, is less than about 254 mm³ at day 1, about 247 mm³ at day 5, about 198 mm³ at day 8, about 113 mm³ at day 13, about 105 mm³ at day 15, about 79 mm³ at day 19, about 72 mm³ at day 22, about 74 mm³ at day 26, about 35 mm³ at day 29, or about 26 mm³ at day 32.

E108. The ADC of any one of E71-E107, wherein the average tumor volume in a mouse SK-129862F(PDX) metastatic melanoma xenograft model wherein the ADC is administered at 10 mg/kg of body weight, every 4^{th} day for 16 days, is less than about 254 mm³ at day 1, about 247 mm³ at day 5, about 198 mm³ at day 8, about 113 mm³ at day 13, about 105 mm³ at day 15, about 79 mm³ at day 19, about 72 mm³ at day 22, about 74 mm³ at day 26, about 35 mm³ at day 29, or about 26 mm³ at day 32, and further wherein the average tumor volume in an otherwise identical mouse wherein the ADC is not administered is less than about 234 mm³ at day 1, about 239 mm³ at day 5, about 237 mm³ at day 8, about 206 mm³ at day 13, about 220 mm³ at day 15, about 211 mm³ at day 19, about 195 mm³ at day 22, about 233 mm³ at day 26, about 253 mm³ at day 29, or about 271 mm³ at day 32.

E109. The ADC of any one of E71-E108, wherein the average tumor volume in a mouse SK-129862F(PDX) metastatic melanoma xenograft model wherein the ADC is administered at 10 mg/kg of body weight, every 4^{th} day for 16 days, is about 162 mm³ to about 254 mm³ at day 1, about 143 mm³ to about 247 mm³ at day 5, about 98 mm³ to about 198 mm³ at day 8, about 69 mm³ to about 113 mm³ at day 13, about 57 mm³ to about 105 mm³ at day 15, about 39 mm³ to about 79 mm³ at day 19, about 24 mm³ to about 72 mm³ at day 22, about 30 mm³ to about 74 mm³ at day 26, about 11 mm³ to about 35 mm³ at day 29, 0 mm³ to about 26 mm³ at day 32

E110. The ADC of any one of E71-E109, wherein the average tumor volume in a mouse SK-129862F(PDX) metastatic melanoma xenograft model wherein the ADC is administered at 10 mg/kg of body weight, every 4^{th} day for 16 days, is about 162 mm³ to about 254 mm³ at day 1, about 143 mm³ to about 247 mm³ at day 5, about 98 mm³ to about 198 mm³ at day 8, about 69 mm³ to about 113 mm³ at day 13, about 57 mm³ to about 105 mm³ at day 15, about 39 mm³ to about 79 mm³ at day 19, about 24 mm³ to about 72 mm³ at day 22, about 30 mm³ to about 74 mm³ at day 26, about 11 mm³ to about 35 mm³ at day 29, 0 mm³ to about 26 mm³ at day 32, and further wherein the average tumor volume in an otherwise identical mouse wherein the ADC is not administered is about 178 mm³ to about 234 mm³ at day 1, about 189 mm³ to about 239 mm³ at day 5, about 159 mm³ to about 237 mm³ at day 8, about 166 mm³ to about 206 mm³ at day 13, about 184 mm³ to about 220 mm³ at day 15, about 169 mm³ to about 211 mm³ at day 19, about 165 mm³ to about 195 mm³ at day 22, about 199 mm³ to about 233 mm³ at day 26, about 213 mm³ to about 253 mm³ at day 29, 233 mm³ to about 271 mm³ at day 32.

E111. The ADC of any one of E71-E110, wherein the terminal plasma half-life in a mouse is at least one or more of about 1 day, about 1.5 days, about 2 days, about 2.5 days, about 3 days, about 3.5 days, about 4 days, about 4.5 days, about 5 days, about 5.5 days and about 5.9 days.

E112. The ADC of any one of E71-E111, wherein the terminal plasma half-life in mice is at least 5.6 days or 5.9 days.

E113. The ADC of any one of E71-E112, wherein the terminal plasma half-life in a rat is at least one or more of about 1 day, about 1.5 days, about 2 days, about 2.5 days, about 3 days, about 3.5 days, about 4 days, about 4.5 days, about 5 days, about 5.5 days, about 6 days, about 6.5 days, about 7 days, about 7.5 days, about 8 days and about 8.5 days.

E114. The ADC of any one of E71-E113, wherein the terminal plasma half-life in a rat is at least 8.1 days or 8.5 days.

E115. The ADC of any one of E71-E114, wherein the terminal plasma half-life in a cynomolgus monkey is at least one or more of about 1 day, about 1.5 days, about 2 days, about 2.5 days, about 3 days, about 3.5 days, about 4 days, about 4.5 days, about 5 days, about 5.5 days, about 6 days, about 6.5 days, about 7 days, about 7.5 days, and about 7.7 days.

E116. The ADC of any one of E71-E115, wherein the terminal plasma half-life in a cynomolgus monkey is at least 7 days, 7.6 days or 7.7 days.

E117. The ADC of any one of E71-E116, wherein the terminal plasma half-life in a human is at least one or more of about 1 day, about 1.5 days, about 2 days, about 2.5 days, about 3 days, about 3.5 days, about 4 days, about 4.5 days, about 5 days, about 5.5 days, about 6 days, about 6.5 days, about 7 days, about 7.5 days, and about 7.7 days.

E118. The ADC of any one of E71-E117, wherein the terminal plasma half-life in a human is at least 7 days, 7.6 days or 7.7 days.

E119. A process for producing the ADC of any one of E71-E118, comprising (a) linking the linker to the drug; (b) conjugating the linker-drug to the antibody; and (c) purifying the ADC.

E120. A pharmaceutical composition comprising the ADC, of any one of E71-E118, and a pharmaceutically acceptable carrier.

E121. A method of treating a disease or disorder associated with GD3 cell surface expression compared with the GD3 cell surface expression in an otherwise identical normal cell, comprising administering to a subject in need thereof a therapeutically effective amount of the ADC, of any one of embodiments E71-E118, or the pharmaceutical composition of E120.

E122. The method of E121, wherein the disease or disorder associated with GD3 cell surface expression is melanoma, breast cancer, glioma, glioblastoma, or lung cancer.

E123. A method of treating a disease or disorder associated with an elevated level of GD3 activity in a cell, comprising administering to a cell having an elevated level of GD3 activity a therapeutically effective amount of the ADC of any one of embodiments E71-E118, or the pharmaceutical composition of E120.

E124. A method of treating a disease or disorder associated with an elevated level of GD3 activity in a subject, comprising administering to a subject in need thereof a therapeutically effective amount of the ADC, of any one of embodiments E71-E118, or the pharmaceutical composition of E120.

E125. The method of E123 or E124, wherein the GD3 activity is selected from the group consisting of: increased cell growth, increased cell division, loss of contact inhibition, increased cell invasion, increased cell adhesion, and increased apoptosis.

E126. The method of any one of E123-E125, wherein the disease or disorder associated with elevated levels of GD3 activity is melanoma, breast cancer, glioma, glioblastoma, or lung cancer.

E127. The method of any one of E121-E126, comprising administering to a subject in need thereof 0.5 mg/kg of the ADC, of any one of embodiments E71-E118, or the pharmaceutical composition of E120.

E128. The method of any one of E121-E127, comprising administering said ADC, or pharmaceutical composition, intravenously.

E129. The method of any one of E121-E128, wherein said ADC, or pharmaceutical composition, is administered about twice a week, once a week, once every two weeks, once every three weeks, once every four weeks, once every five weeks, once every six weeks, once every seven weeks, once every eight weeks, once every nine weeks, once every ten weeks, twice a month, once a month, once every two months, once every three months, or once every four months.

E130. The ADC of any one of E71-E118, or the pharmaceutical composition of E120, for use as a medicament.

### BRIEF DESCRIPTION OF THE DRAWINGS

FIG. 1 provides the heavy chain sequence of mR24 and the sequences of several humanized heavy chain variable domain (VH) variants, numbered 1.0 through 1.8, of antibody mR24. Numbering is for the linear sequence. Differences in the sequences of humanized variants 1.0 through 1.8 from the sequence of mR24 are underlined. These differences are due to different residues present in the framework chosen for humanization. Mutations introduced into the sequences of humanized variants 1.0 through 1.8 are shown as bold. Mutations introduce into VH variants 1.6 and 1.7 removed a homotypic interface important to binding GD3, and resulted in significant loss of activity as described in Examples 2, Example 3, and table 5 below. The A_H74_P mutation introduce into VH variant 1.1 resulted in increased activity, when compared to the other mR24VH variants, as described in Example 2, Example 3, and table 5 below. FIG. 1 discloses SEQ ID NOS 16, 30, 1, 38, 39, 35, and 40-42, respectively, in order of appearance.

FIG. 2 provides the light chain sequence of mR24 and the sequences of several light chain variable domain (VL) variants, numbered 1.0 through 1.8, of antibody mR24. Numbering is for the linear sequence. Differences in the sequences of humanized variants 1.0 through 1.8 from mR24 are underlined. Mutations introduced into the sequences of humanized variants 1.0 through 1.8 are shown as bold. The S_L65_W mutation introduce into VL variant 1.2 resulted in increased activity, when compared to the other mR24VL variants, as described in Example 2, Example 3, and table 5 below. FIG. 2 discloses SEQ ID NOS 18, 36-37, 9, and 43-48, respectively, in order of appearance.

FIG. 3 shows the cross-species identity of Light Chain Residue 65 (according to Kabat numbering). The relative frequency of each natural amino acid residue (abbreviated by single letter code) at Kabat light chain position 65, where numbering is according to the linear sequence, is shown for human, murine, and all other species (e.g. rabbit, pig, chicken, and rat) in the Abysis database. Selection pressure for Serine at Kabat light chain position 65 is evident.

FIG. 4 shows a structural alignment of the chimeric mR24 Fab heavy chain and the huR24 VH1.0/VL1.0 homology model heavy chain (shown in black). All residues are labeled according to Kabat numbering. The structural alignment shows that a mutation at position H74 from alanine to proline could have an effect on the position and rigidity of the loop containing residues H71 and H73, which interact at the junction of CDR-H1 and CDR-H2. For further discussion, see Example 3 below.

FIG. 5 provides an analysis of a plate based ELISA binding assay demonstrating comparable binding of huR24 vh1.1/vk1.2 and chR24 to GD3 directly immobilized on the ELISA plate.

FIG. 6A provides an analysis of a cell surface binding assay demonstrating comparable binding of huR24 vh1.1/vk1.2 and mR24 (using chimeric chR24) to G361 tumor cells overexpressing GD3. The G361 cells were grown in the wells of an ELISA plate, and the antibodies were added to the plate followed by washing and detection of bound antibodies using Horseradish-Peroxidase (HRP)-conjugated, goat anti-human IgG antibodies.

FIG. 6B shows a graph depicting an analysis of a cell surface binding assay demonstrating comparable binding of huR24 and mR24 (using chimeric chR24) to SK-MEL028 tumor cells overexpressing GD3. The SK-MEL028 cells were grown in the wells of an ELISA plate, and the antibodies were added to the plate followed by washing and detection of bound antibodies using Horseradish-Peroxidase (HRP)-conjugated, goat anti-human IgG antibodies.

FIGS. 7A-7F show graphs depicting results of a flow cytometry binding assay demonstrating specific cell surface binding of to GD3-positive human melanoma cell lines: SK-MEL-28 (FIG. 7A), G361 (FIG. 7B), SK-MEL-30 (FIG. 7C), MeWo (FIG. 7D), Malme-3M (FIG. 7E), and COLO-205 (FIG. 7F).

FIG. 8A shows a graph depicting an analysis of huR24 and huR24-ADC binding to cell surface GD3 on Malme-3M human melanoma cells and subsequent internalization. An imaging flow cytometry-based method to measure internalization was used to determine the internalization of huR24 and huR24-ADC molecules into the melanoma cells. To quantitate co-localization between internalized anti-GD3 and the lysosome, samples were incubated with huR24 or huR24-ADC, stained with a fluorescently labeled anti-LAMP-1 that localizes the lysosomal marker LAMP-1. Co-localization of the GD3-antibody and the GD3-ADC with the LAMP-1 lysosomal marker proceeded with indistinguishable kinetics based on a calculated similarity score. Surprisingly, huR24-ADC consistently demonstrated the ability to internalize and remain in the cell to an even higher degree than huR24, as evidenced by its similarity score of about 0.9 to 1.1.

FIG. 8B provides an analysis of huR24 and huR24-ADC binding to cell surface GD3 on SK-MEL-28 human melanoma cells and subsequent internalization. An imaging flow cytometry-based method to measure internalization was used to determine the internalization of huR24 and huR24-ADC molecules into the melanoma cells. To quantitate co-localization between internalized anti-GD3 and the lysosome, samples were incubated with huR24 or huR24-ADC, stained with a fluorescently labeled anti-LAMP-1 that localizes the lysosomal marker LAMP-1. Co-localization of the GD3-antibody and the GD3-ADC with the LAMP-1 lysosomal marker proceeded with indistinguishable kinetics based on a calculated similarity score. Surprisingly, huR24-ADC consistently demonstrated the ability to internalize and remain in the cell to an even higher degree than huR24, as evidenced by its similarity score of about 0.9 to 1.1.

FIG. 9 provides data on huR24-ADC cell binding to human and cynomolgus monkey cells. The data shown demonstrate that huR24-ADC binds normal monkey dermal fibroblasts, human dermal fibroblasts and monkey melanocytes more than a control ADC. In contrast, huR24-ADC binds human epidermal melanocytes (i.e. HEMa-LP melanocytes and HEMn-melanocytes) expressing increased level of GD3 compared to cell expressing GD3 at a lower level, to a much greater extent than the control ADC. These data demonstrate that huR24 selectively binds melanocyte cell lines expressing increased levels of GD3 to a greater extent than cells expressing GD3 at lower levels. See Example 8 below for further discussion.

FIGS. 10A-10E depict human and cynomolgus monkey cell cytotoxicity data for huR24-ADC compared with a negative control ADC. huR24-ADC showed a similar cytotoxicity profile in human cells and cynomolgus monkey cells (FIGS. 10A and 10B). In human epidermal melanocytes, huR24-ADC showed markedly increased cell killing (FIGS. 10C and 10D). huR24-ADC also showed cell killing in cynomolgus monkey melanocytes (FIG. 10E). Considered with the data presented in FIG. 9, these data indicate huR24-ADC cell killing in a concentration-dependent manner that was also correlated with the level of cell surface GD3 expression. These data confirm that huR24-ADC was a highly selective cytotoxic agent that selectively kills cells which express surface GD3, indicating it is a potential novel therapeutic for that disease, as demonstrated in Example 8 below.

FIG. 11A provides human melanoma xenograft growth curves in a SK-MEL-19 xenograft model. The up arrows (T) indicate dosing of the control PBS, control ADC at 6 mg/kg, and huR24-ADC at 3, 6, and 10 mg/kg, respectively. The data demonstrate the decrease in SK-MEL-19 tumor volume (expressed as cubic millimeters mm³) after dosing on days 0, 4, 8 and 12. Reduction of tumor volume by huR24-ADC at 3 or 6 mg/kg was not substantially greater than reduction of tumor volume by control ADC at 6 mg/kg. However, the reduction of tumor volume by huR24-ADC at 10 mg/kg was significantly enhanced. These data indicate that huR24-ADC is a selective inhibitor of tumor growth in art-recognized in vivo tumor models.

FIG. 11B provides human melanoma xenograft growth curves in a SK-129862F patient derived xenograft (PDX) model. The up arrows (T) indicates dosing of the control PBS, control ADC at 6 mg/kg, and huR24-ADC at 3, 6, and 10 mg/kg, respectively. The data demonstrate the decrease in SK-129862F (PDX) tumor volume (expressed as cubic millimeters mm³) after dosing on days 0, 4, 8 and 12. Reduction of tumor volume by huR24-ADC at 3 mg/kg was not substantially greater than reduction of tumor volume by control ADC at 6 mg/kg. However, the reduction of tumor volume by huR24-ADC at 6 mg/kg was significantly enhanced compared with control ADC and even greater difference in tumor cell volume reduction (as a measure of tumor growth) was evident for huR24-ADC at 10 mg/kg. These data indicate that huR24-ADC is a selective inhibitor of tumor growth in art-recognized in vivo tumor models.

FIG. 12 depicts the structure of human GD3.

### DETAILED DESCRIPTION OF THE INVENTION

The present invention provides ADCs that bind to GD3. The invention also provides processes for preparing the conjugates using GD3 antibodies, linkers, and drugs. The ADCs of the invention are useful for the preparation and manufacture of compositions, such as medicaments that may be used in the diagnosis, prophylaxis, and/or treatment of hyperproliferative disorders characterized by GD3 expression.

### Antibodies

An “antibody” or “Ab” is an immunoglobulin molecule capable of recognizing and binding to a specific target or antigen, such as a carbohydrate, polynucleotide, lipid, polypeptide, etc., through at least one antigen recognition site, located in the variable region of the immunoglobulin molecule. As used herein, the term “antibody” can encompass any type of antibody, including but not limited to monoclonal antibodies, polyclonal antibodies, antigen-binding fragments (or portion), of intact antibodies that retain the ability to specifically bind to a given antigen (e.g. GD3).

An “antigen-binding fragment” of an antibody refers to a fragment of a full-length antibody that retains the ability to specifically bind to an antigen (preferably with substantially the same binding affinity). Examples of an antigen-binding fragment includes (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fv fragment consisting of the VH and CH1 domains; (iv) a Fv fragment consisting of the VL and VH domains of a single arm of an antibody, (v) a dAb fragment (Ward et al., 1989 Nature 341:544-546), which consists of a VH domain; and (vi) an isolated complementarity determining region (CDR), disulfide-linked Fvs (dsFv), and anti-idiotypic (anti-Id) antibodies and intrabodies. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv)); see e.g., Bird et al. Science 242:423-426 (1988) and Huston et al., 1988, Proc. Natl. Acad. Sci. USA 85:5879-5883. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen-binding sites (see e.g., Holliger et al, 1993, Proc. Natl. Acad. Sci. USA 90:6444-6448; Poljak et al., 1994, Structure 2:1121-1123). Other forms of single chain antibodies, such as maxibodies, minibodies, intrabodies, triabodies, tetrabodies, v-NAR and bis-scFv are also encompassed (see, e.g., Hollinger and Hudson, 2005, Nature Biotechnology 23(9): 1126-1136).

An antibody “variable domain” refers to the variable region of the antibody light chain (VL) or the variable region of the antibody heavy chain (VH), either alone or in combination. As known in the art, the variable regions of the heavy and light chains each consist of four framework regions (FR) connected by three complementarity determining regions (CDRs), and contribute to the formation of the antigen-binding site of antibodies.

“Complementarity Determining Regions” (CDRs) can be identified according to the definitions of the Kabat, Chothia, the accumulation of both Kabat and Chothia, AbM, contact, North, and/or conformational definitions or any method of CDR determination well known in the art. See, e.g., Kabat et al., 1991, Sequences of Proteins of Immunological Interest, 5th ed. (hypervariable regions); Chothia et al., 1989, Nature 342:877-883 (structural loop structures). The identity of the amino acid residues in a particular antibody that make up a CDR can be determined using methods well known in the art. AbM definition of CDRs is a compromise between Kabat and Chothia and uses Oxford Molecular's AbM antibody modeling software (Accelrys®). The “contact” definition of CDRs is based on observed antigen contacts, set forth in MacCallum et al., 1996, J. Mol. Biol., 262:732-745. The “conformational” definition of CDRs is based on residues that make enthalpic contributions to antigen binding (see, e.g., Makabe et al., 2008, J. Biol. Chem., 283:1156-1166). North has identified canonical CDR conformations using a different preferred set of CDR definitions (North et al., 2011, J. Mol. Biol. 406: 228-256). In another approach, referred to herein as the “conformational definition” of CDRs, the positions of the CDRs may be identified as the residues that make enthalpic contributions to antigen binding (Makabe et al., 2008, J Biol. Chem. 283:1156-1166). Still other CDR boundary definitions may not strictly follow one of the above approaches, but will nonetheless overlap with at least a portion of the Kabat CDRs, although they may be shortened or lengthened in light of prediction or experimental findings that particular residues or groups of residues or even entire CDRs do not significantly impact antigen binding. As used herein, a CDR may refer to CDRs defined by any approach known in the art, including combinations of approaches. The methods used herein may utilize CDRs defined according to any of these approaches. For any given embodiment containing more than one CDR, the CDRs (or other residue of the antibody) may be defined in accordance with any of Kabat, Chothia, North, extended, AbM, contact, and/or conformational definitions.

Residues in a variable domain are numbered according Kabat, which is a numbering system used for heavy chain variable domains or light chain variable domains of the compilation of antibodies. See, Kabat et al., 1991, Sequences of Proteins of Immunological Interest, 5th Ed. Public Health Service, National Institutes of Health, Bethesda, Md. Using this numbering system, the actual linear amino acid sequence may contain fewer or additional amino acids corresponding to a shortening of, or insertion into, a FR or CDR of the variable domain. For example, a heavy chain variable domain may include a single amino acid insert (residue 52a according to Kabat) after residue 52 of H2 and inserted residues (e.g. residues 82a, 82b, and 82c, according to Kabat) after heavy chain FR residue 82. The Kabat numbering of residues may be determined for a given antibody by alignment at regions of homology of the sequence of the antibody with a “standard” Kabat numbered sequence. Various algorithms for assigning Kabat numbering are available. The algorithm implemented in the version 2.3.3 release of Abysis (www.abysis.org) is used herein to assign Kabat numbering to variable regions CDR-L1, CDR-L2, CDR-L3, CDR-H2, and CDR-H3. AbM definition is used for CDR-H1.

“Framework” (FR) residues are antibody variable domain residues other than the CDR residues. A VH or VL domain framework comprises four framework sub-regions, FR1, FR2, FR3 and FR4, interspersed with CDRs in the following structure: FR1-CDR1-FR2-CDR2-FR3-CDR3-FR4.

In certain embodiments, the antibody, or antigen-binding fragment thereof, described herein comprises an Fc domain. The Fc domain can be derived from IgA (e.g., IgA₁ or IgA₂), IgD, IgE, IgM, or IgG (e.g., IgG₁, IgG₂, IgG₃, or IgG₄).

An “Fc fusion” protein is a protein wherein one or more polypeptides are operably linked to an Fc polypeptide. An Fc fusion combines the Fc region of an immunoglobulin with a fusion partner.

An “epitope” refers to the area or region of an antigen to which an antibody specifically binds, e.g., an area or region comprising residues that interacts with the antibody. Epitopes can be linear or conformational.

An antibody that “preferentially binds” or “specifically binds” (used interchangeably herein) to an epitope is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to a GD3 epitope is an antibody that binds this epitope with greater affinity, avidity, more readily, and/or with greater duration than it binds to other GD3 epitopes or non-GD3 epitopes. It is also understood by reading this definition that, for example, an antibody (or moiety or epitope) which specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding. “Specific binding” or “preferential binding” includes a compound, e.g., a protein, a nucleic acid, an antibody, and the like, which recognizes and binds to a specific molecule, but does not substantially recognize or bind other molecules in a sample. For instance, an antibody or a peptide receptor which recognizes and binds to a cognate ligand or binding partner (e.g., an anti-human tumor antigen antibody that binds a tumor antigen) in a sample, but does not substantially recognize or bind other molecules in the sample, specifically binds to that cognate ligand or binding partner. Thus, under designated assay conditions, the specified binding moiety (e.g., an antibody or an antigen-binding portion thereof or a receptor or a ligand binding portion thereof) binds preferentially to a particular target molecule and does not bind in a significant amount to other components present in a test sample.

A variety of assay formats may be used to select an antibody or peptide that specifically binds a molecule of interest. For example, solid-phase ELISA immunoassay, immunoprecipitation, BIAcore™ (GE Healthcare, Piscataway, N.J.), fluorescence-activated cell sorting (FACS), Octet™ (ForteBio, Inc., Menlo Park, Calif.) and Western blot analysis are among many assays that may be used to identify an antibody that specifically reacts with an antigen or a receptor, or ligand binding portion thereof, that specifically binds with a cognate ligand or binding partner. Typically, a specific or selective reaction will be at least twice background signal or noise and more typically more than 10 times background, even more specifically, an antibody is said to “specifically bind” an antigen when the equilibrium dissociation constant (K_{D}) is ≤1 μM, preferably ≤100 nM, more preferably ≤10 nM, even more preferably, ≤100 pM, yet more preferably, ≤10 pM, and even more preferably, ≤1 pM.

The antibody, or antigen-binding fragment thereof, of the invention may be “affinity matured” using standard techniques well-known in the art For example, an affinity matured antibody can be produced by procedures known in the art (Marks et al., 1992, Bio/Technology, 10:779-783; Barbas et al., 1994, Proc Nat. Acad. Sci, USA 91:3809-3813; Schier et al., 1995, Gene, 169:147-155; Yelton et al., 1995, J. Immunol., 155:1994-2004; Jackson et al., 1995, J. Immunol., 154(7):3310-9; Hawkins et al., 1992, J. Mol. Biol., 226:889-896; and WO2004/058184).

The term “compete”, as used herein with regard to an antibody, means that binding of a first antibody, or an antigen-binding portion thereof, to an antigen reduces the subsequent binding of the same antigen by a second antibody or an antigen-binding portion thereof. In general, the binding a first antibody creates steric hindrance, conformational change, or binding to a common epitope (or portion thereof), such that the binding of the second antibody to the same antigen is reduced. Standard competition assays may be used to determine whether two antibodies compete with each other. One suitable assay for antibody competition involves an ELISA-based approach the use of the Biacore technology, which can measure the extent of interactions using surface plasmon resonance (SPR) technology, typically using a biosensor system (such as a BIACORE® system). For example, SPR can be used in an in vitro competitive binding inhibition assay to determine the ability of one antibody to inhibit the binding of a second antibody. Another assay for measuring antibody competition uses the Biacore technology, which can measure the extent of interactions using surface plasmon resonance (SPR) technology, typically using a biosensor system (such as a BIACORE® system). For example, SPR can be used in an in vitro competitive binding inhibition assay to determine the ability of one antibody to inhibit the binding of a second antibody.

Furthermore, a high throughput process for “binning” antibodies based upon their competition is described in International Patent Application No. WO2003/48731. Competition is present if one antibody (or fragment) reduces the binding of another antibody (or fragment) to GD3. For example, a sequential binding competition assay may be used, with different antibodies being added sequentially. The first antibody may be added to reach binding that is close to saturation. Then, the second antibody is added. If the binding of second antibody to GD3 is not detected, or is significantly reduced (e.g., at least about 10%, at least about 20%, at least about 30%, at least about 40%, at least about 50%, at least about 60%, at least about 70%, at least about 80%, or at least about 90% reduction) as compared to a parallel assay in the absence of the first antibody (which value can be set as 100%), the two antibodies are considered as competing with each other.

In a process known as “germlining”, certain amino acids in the VH and VL sequences can be mutated to match those found naturally in germline VH and VL sequences. In particular, the amino acid sequences of the framework regions in the VH and VL sequences can be mutated to match the germline sequences to reduce the risk of immunogenicity when the antibody is administered. As used herein, the term “germline” refers to the nucleotide sequences and amino acid sequences of the antibody genes and gene segments as they are passed from parents to offspring via the germ cells. This germline sequence is distinguished from the nucleotide sequences encoding antibodies in mature B cells which have been altered by recombination and hypermutation events during the course of B cell maturation. An antibody that “utilizes” a particular germline has a nucleotide or amino acid sequence that most closely aligns with that germline nucleotide sequence or with the amino acid sequence that it specifies. Such antibodies frequently are mutated compared with the germline sequence. Germline DNA sequences for human VH and VL genes are known in the art (see e.g., the “Vbase” human germline sequence database; see also Kabat, E. A., et al., 1991, Sequences of Proteins of Immunological Interest, Fifth Edition, U.S. Department of Health and Human Services, NIH Publication No. 91-3242; Tomlinson et al., J. Mol. Biol. 227:776-798, 1992; and Cox et al., Eur. J. Immunol. 24:827-836, 1994.)

The term “treatment” includes prophylactic and/or therapeutic treatments. If it is administered prior to clinical manifestation of a condition, the treatment is considered prophylactic. Therapeutic treatment includes, e.g., ameliorating or reducing the severity of a disease, or shortening the length of the disease.

### Binding Affinity

The binding affinity of an antibody can be expressed as K_{D} value, which refers to the dissociation rate of a particular antigen-antibody interaction. K_{D} is the ratio of the rate of dissociation, also called the “off-rate (k_{off})”, to the association rate, or “on-rate (kₒₙ)”. Thus, K_{D} equals k_{off}/kₒₙ and is expressed as a molar concentration (M), and the smaller the K_{D}, the stronger the affinity of binding. K_{D} values for antibodies can be determined using methods well established in the art. One exemplary method for measuring K_{D} is surface plasmon resonance (SPR), typically using a biosensor system such as a BIACORE® system. BIAcore kinetic analysis comprises analyzing the binding and dissociation of an antigen from chips with immobilized molecules (e.g. molecules comprising epitope binding domains), on their surface. Another method for determining the K_{D} of an antibody is by using Bio-Layer Interferometry, typically using OCTET® technology (Octet QKe system, ForteBio). Alternatively or in addition, a KinExA® (Kinetic Exclusion Assay) assay, available from Sapidyne Instruments (Boise, Id.) can also be used.

### Humanization

While humanized antibodies are desirable because of their potential low immunogenicity in humans, their production is unpredictable. For example, sequence modification to reduce potential immunogenicity may have unintended and unpredictable effects on other aspects of antibody function, such as binding, binding specificity, clearance, PK, PD, stability, viscosity, aggregation, folding, and so on. Furthermore, “humanized antibodies” may still exhibit immunogenicity in humans, irrespective of sequence modification.

As used herein, “humanized” or “CDR grafted” antibodies refer to forms of non-human (e.g. murine) antibodies that are chimeric immunoglobulins, immunoglobulin chains, or fragments thereof (such as Fv, Fab, Fab′, F(ab′)₂ or other antigen binding subsequences of antibodies) that contain minimal sequence derived from a non-human immunoglobulin. Preferably, humanized antibodies are human immunoglobulins (recipient antibody) in which residues from one or more complementary determining regions (CDRs) of the recipient are replaced by residues from one or more CDRs of a non-human species (donor antibody) such as mouse, rat, or rabbit having the desired specificity, affinity, and capacity.

In some instances, Fv framework region (FR) residues of the human immunoglobulin are replaced by corresponding non-human residues. Furthermore, the humanized antibody may include residues that are found neither in the recipient antibody nor in the imported CDR or framework sequences, but are included to further refine and optimize antibody performance. In general, the humanized antibody will include substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will include at least a portion of an immunoglobulin constant region or domain (Fc), typically that of a human immunoglobulin. In some aspects of the invention the antibodies have Fc regions modified as described in PCT International Publication No. WO 99/58572. Other forms of humanized antibodies have one or more CDRs (CDR L1, CDR L2, CDR L3, CDR H1, CDR H2, or CDR H3) which may be altered with respect to the original antibody, which are also termed one or more CDRs “derived from” one or more CDRs from the original antibody.

Humanization can be essentially performed following the method of Winter and co-workers (Jones et al. Nature 321:522-525 (1986); Riechmann et al. Nature 332:323-327 (1988); Verhoeyen et al. Science 239:1534-1536 (1988)), by substituting rodent or mutant rodent CDRs or CDR sequences for the corresponding sequences of a human antibody. See also U.S. Pat. Nos. 5,225,539; 5,585,089; 5,693,761; 5,693,762; 5,859,205; which are incorporated herein by reference in its entirety. In some instances, residues within the framework regions of one or more variable regions of the human immunoglobulin are replaced by corresponding non-human residues (see, for example, U.S. Pat. Nos. 5,585,089; 5,693,761; 5,693,762; and 6,180,370). Furthermore, humanized antibodies may include residues that are not found in the recipient antibody or in the donor antibody. These modifications are made to further refine antibody performance (e.g., to obtain a desired affinity, specificity, and the like). In general, the humanized antibody will include substantially all of at least one, and typically two, variable domains, in which all or substantially all of the hypervariable regions correspond to those of a non-human immunoglobulin and all or substantially all of the framework regions are those of a human immunoglobulin sequence. The humanized antibody optionally also will include at least a portion of an immunoglobulin constant region (Fc), typically that of a human immunoglobulin. For further details see Jones et al. Nature 331:522-525 (1986); Riechmann et al. Nature 332:323-329 (1988); and Presta Curr. Op. Struct. Biol. 2:593-596 (1992); which are incorporated herein by reference in its entirety. Accordingly, such “humanized” antibodies may include antibodies wherein substantially less than an intact human variable domain has been substituted by the corresponding sequence from a non-human species. In practice, humanized antibodies are typically human antibodies in which some CDR residues and possibly some framework residues are substituted by residues from analogous sites in rodent antibodies. See, for example, U.S. Pat. Nos. 5,225,539; 5,585,089; 5,693,761; 5,693,762; 5,859,205. See also U.S. Pat. No. 6,180,370, and PCT International Publication No. WO 01/27160, where humanized antibodies and techniques for producing humanized antibodies having improved affinity for a predetermined antigen are disclosed.

“Recombinant human antibody” or “fully human antibody” refers to those antibodies having an amino acid sequence corresponding to that of an antibody produced by a human and/or which has been made using any of the techniques for making human antibodies known to those skilled in the art or disclosed herein. This definition of a human antibody includes antibodies having at least one human heavy chain polypeptide or at least one human light chain polypeptide. One such example is an antibody having murine light chain and human heavy chain polypeptides. Human antibodies can be produced using various techniques known in the art. For example, a human antibody is selected from a phage library, where that phage library expresses human antibodies (Vaughan et al., Nature Biotechnology, 14:309-314, (1996); Sheets et al., Proc. Natl. Acad. Sci. (USA) 95:6157-6162, (1998); Hoogenboom and Winter, J. Mol. Biol., 227:381, (1991); Marks et al., J. Mol. Biol., 222:581, (1991)). Human antibodies can also be made by immunization of animals into which human immunoglobulin loci have been transgenically introduced in place of the endogenous loci, e.g., mice in which the endogenous immunoglobulin genes have been partially or completely inactivated. This approach is described in U.S. Pat. Nos. 5,545,807; 5,545,806; 5,569,825; 5,625,126; 5,633,425; and 5,661,016. Alternatively, the human antibody may be prepared by immortalizing human B lymphocytes that produce an antibody directed against a target antigen (such B lymphocytes may be recovered from an individual or from single cell cloning of the cDNA, or may have been immunized in vitro). See, e.g., Cole et al. Monoclonal Antibodies and Cancer Therapy, Alan R. Liss, p. 77, (1985); Boerner et al., J. Immunol., 147 (1):86-95, (1991); and U.S. Pat. No. 5,750,373.

In general, for the production of hybridoma cell lines, the route and schedule of immunization of the host animal are generally in keeping with established and conventional techniques for antibody stimulation and production. It is contemplated that any mammalian subject including humans or antibody producing cells therefrom can be manipulated to serve as the basis for production of mammalian, including human and hybridoma cell lines. Typically, the host animal is inoculated intraperitoneally, intramuscularly, orally, subcutaneously, intraplantar, and/or intradermally with an amount of immunogen, including as described herein.

Hybridomas can be prepared from the lymphocytes and immortalized myeloma cells using the general somatic cell hybridization technique of Kohler, B. and Milstein, C., Nature 256:495-497, 1975 or as modified by Buck, D. W., et al., In Vitro, 18:377-381, 1982. Available myeloma lines, including but not limited to X63-Ag8.653 and those from the Salk Institute, Cell Distribution Center, San Diego, Calif., USA, may be used in the hybridization. Generally, the technique involves fusing myeloma cells and lymphoid cells using a fusogen such as polyethylene glycol, or by electrical means, all as well known to those skilled in the art. Hybridomas that may be used as source of antibodies encompass all derivatives, progeny cells of the parent hybridomas that produce monoclonal antibodies specific for GD3, or a portion thereof.

Hybridomas that produce such antibodies may be grown in vitro or in vivo using known procedures. The monoclonal antibodies may be isolated from the culture media or body fluids, by conventional immunoglobulin purification procedures such as ammonium sulfate precipitation, gel electrophoresis, dialysis, chromatography, and ultrafiltration, if desired.

Alternatively, the polynucleotide sequence encoding an antibody may be used for genetic manipulation to “humanize” the antibody or to improve the affinity, or other characteristics of the antibody. For example, the constant region may be engineered to more nearly resemble human constant regions to avoid immune response if the antibody is used in clinical trials and treatments in humans. It may be desirable to genetically manipulate the antibody sequence to obtain greater affinity to GD3 and greater efficacy in inhibiting GD3.

Humanized antibodies may be prepared using any one of a variety of methods including veneering, grafting of complementarity determining regions (CDRs), grafting of abbreviated CDRs, grafting of specificity determining regions (SDRs), and Frankenstein assembly, as described below. Humanized antibodies also include superhumanized antibodies, in which one or more changes have been introduced in the CDRs. For example, human residues may be substituted for non-human residues in the CDRs. These general approaches may be combined with standard mutagenesis and synthesis techniques to produce an anti-GD3 antibody of any desired sequence.

Veneering is based on the concept of reducing potentially immunogenic amino acid sequences in a rodent or other non-human antibody by resurfacing the solvent accessible exterior of the antibody with human amino acid sequences. Thus, veneered antibodies appear less foreign to human cells than the unmodified non-human antibody. See Padlan (1991) Mol. Immunol. 28:489-98. A non-human antibody is veneered by identifying exposed exterior framework region residues in the non-human antibody, which are different from those at the same positions in framework regions of a human antibody, and replacement of the identified residues with amino acids that typically occupy these same positions in human antibodies.

Grafting of CDRs is performed by replacing one or more CDRs of an acceptor antibody (e.g., a human antibody or other antibody having desired framework residues) with CDRs of a donor antibody (e.g., a non-human antibody). Acceptor antibodies may be selected based on similarity of framework residues between a candidate acceptor antibody and a donor antibody. For example, human framework regions are identified as having substantial sequence homology to each framework region of the relevant non-human antibody, and CDRs of the non-human antibody are grafted onto the composite of the different human framework regions. A related method also useful for preparation of antibodies of the invention is described in U.S. Patent Application Publication No. 2003/0040606.

Grafting of abbreviated CDRs is a related approach. Abbreviated CDRs include the specificity-determining residues and adjacent amino acids, including those at positions 27d-34, 50-55 and 89-96 in the light chain, and at positions 31-35b, 50-58, and 95-101 in the heavy chain (numbering convention of (Kabat et al. (1987)). See (Padlan et al., 1995, FASEB J. 9: 133-139). Grafting of specificity-determining residues (SDRs) is premised on the understanding that the binding specificity and affinity of an antibody combining site is determined by the most highly variable residues within each of the complementarity determining regions (CDRs). Analysis of the three-dimensional structures of antibody-antigen complexes, combined with analysis of the available amino acid sequence data may be used to model sequence variability based on structural dissimilarity of amino acid residues that occur at each position within the CDR. SDRs are identified as minimally immunogenic polypeptide sequences consisting of contact residues. See Padlan et al. 1995.

In general, human acceptor frameworks are selected on the basis that they are substantially similar to the framework regions of the donor antibodies, or which are most similar to the consensus sequence of the variable region subfamily. Following grafting, additional changes may be made in the donor and/or acceptor sequences to optimize antibody binding, functionality, codon usage, expression levels, etc., including introduction of non-human residues into the framework regions. See e.g., PCT International Publication No. WO 91/09967.

In some aspects, the VL framework is DPK9. Other similar framework regions are also predicted to deliver advantageous antibodies of the invention comprising CDRs of SEQ ID NO: 9, including DPK5, DPK4, DPK1, IGKV1-5*01, DPK24, DPK21, DPK15, IGKV1-13*02, IGKV1-17*01, DPK8, IGKV3-11*01, and DPK22 which comprise 99, 97, 97, 96, 80, 76, 66, 97, 97, 96, 76, and 74% identity respectively to the FW region of DPK-9 and one or fewer amino acid differences in common structural features (Kabat Numbering) (A) residues directly underneath CDR (Vernier Zone), L2, L4, L35, L36, L46, L47, L48, L49, L64, L66, L68, L69, L71, (B) VH/VL Chain packing Residues: L36, L38, L44, L46, L87 and (C) canonical CDR Structural support residues L2, L48, L64, L71 (see Lo, “Antibody Humanization by CDR Grafting”, (2004) Antibody Engineering, Vol. 248, Methods in Molecular Biology pp 135-159 and O'Brien and Jones, “Humanization of Monoclonal Antibodies by CDR Grafting”, (2003) Recombinant Antibodies for Cancer Therapy, Vol. 207, Methods in Molecular Biology pp 81-100). In some aspects, the frameworks are the framework regions of DPK5, DPK4, DPK1, IGKV1-5*01, DPK24, DPK21, DPK15 sharing 99, 97, 97, 96, 80, 76, 66% identity to DPK9 respectively and have no amino acid differences in these common structural features. In other aspects, the % identity is based on similarity with VL excluding those portions herein defined as CDRs. In some aspects, the VL framework similar to DPK9 comprises a serine to tryptophan mutation at position 65 of the variable region of the light chain (S_L65_W), using Kabat numbering.

In some aspects, the VH framework is DP-54. Other similar framework regions are also predicted to deliver advantageous antibodies of the invention comprising CDRs of SEQ ID NO: 1, including DP-50, IGHV3-30*09, IGHV3-30*15, IGHV3-48*01, DP-77, DP-51, IGHV3-66*01, DP-53, DP-48, IGHV3-53*01, IGHV3-30*02, and DP-49 which comprise 93, 92, 92, 99, 97, 97, 96, 96, 94, 94, 93, 92% identity respectively to the FW region of DP-54 and one or fewer amino acid differences in common structural features (Kabat Numbering) (A) residues directly underneath CDR (Vernier Zone), H2, H47, H48, and H49, H67, H69, H71, H73, H93, H94, (B) VH/VL Chain packing Residues: H37, H39, H45, H47, H91, H93 and (C) canonical CDR Structural support residues H24, H71, H94 (see Lo 2004, supra, and O'Brien and Jones 2003, supra). In some aspects, the frameworks are the framework regions of DP-50, IGHV3-30*09, IGHV3-30*15 sharing 93, 92 and 92% identity to DP-54 respectively and have no amino acid differences in these common structural features. In other aspects, the % identity is based on similarity with VH excluding those portions herein defined as CDRs. In some aspects, the VH framework similar to DP-54 comprises an alanine to proline mutation at position 74 of the variable region of the heavy chain (A_H74_P), using Kabat numbering.

Antigen-binding fragments or antibody fragments can be produced by proteolytic or other degradation of the antibodies, by recombinant methods (i.e., single or fusion polypeptides) as described above or by chemical synthesis. Polypeptides of the antibodies, especially shorter polypeptides up to about 50 amino acids, are conveniently made by chemical synthesis. Methods of chemical synthesis are known in the art and are commercially available. For example, an antibody or antibody fragment could be produced by an automated polypeptide synthesizer employing the solid phase method. See also, U.S. Pat. Nos. 5,807,715; 4,816,567; and 6,331,415.

### Antibodies to GD3

In some aspects, the invention provides antagonistic GD3 antibodies. A high affinity antagonist antibody of the GD3 pathway may be effective on multiple cell types, and multiple tissue compartments, where GD3 is thought to act on its target cells. Antibodies of the invention have the potential to modify an important pathway that drives the development and progression of cancers, including, but not limited to, malignant melanoma, since expression of GD3 by a cell has been shown to be associated with, or involved in, abnormal cell growth and/or division when compared to otherwise an identical normal cell not expressing, or expressing less, GD3.

A “neutralizing” or “blocking” or “antagonist” GD3 antibody, as the terms are used interchangeably herein, refers to an antibody that binds to GD3 and thereby (i) interferes with, limits, reduces or inhibits the interaction between GD3 and a GD3 receptor component (for example, the c-MET signaling pathway); and/or (ii) results in inhibition of at least one biological function of GD3.

“Biological function” or “biological activity” of GD3 is meant to include increased cell growth, increased cell division, and loss of contact inhibition, increased cell invasion, increased cell adhesion, and increased apoptosis.

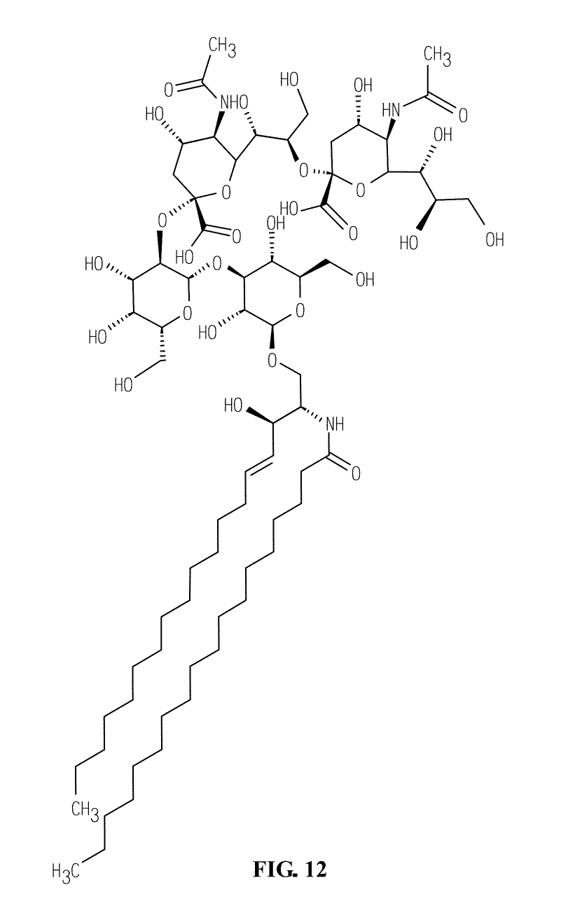

As used herein, the term “GD3” includes variants, isoforms, homologs, orthologs and paralogs of human ganglioside GD3 (e.g., structure shown in FIG. 12). In some aspects of the invention, the antibodies cross-react with GD3 from species other than human, such as GD3 of mouse, rat, or non-human primate, as well as different forms of GD3. In other aspects, the antibodies may be completely specific for human GD3 and may not exhibit species or other types of cross-reactivity. As used herein the term GD3 refers to naturally occurring human GD3 unless contextually dictated otherwise. Therefore, a “GD3 antibody”, “anti-GD3 antibody” or other similar designation means any antibody (as defined herein) that specifically associates, binds or reacts with the GD3 type ligand or isoform, or fragment or derivative thereof.

In some aspects, the GD3 is human GD3. In some aspects, the GD3 is rat GD3. In some aspects, the GD3 is mouse GD3. In some aspects, the GD3 is primate GD3. In some aspects, the GD3 is ape GD3. In some aspects, the GD3 is monkey GD3. In some aspects, the GD3 is cynomolgus monkey GD3. In some aspects GD3 is defined by the chemical structure: Neu5Acα2,8NeuAcα2,3Galβ1,4Glcβ1Cer (Haji-Ghassemi et al., 2015, 25(9):920-952). In some aspects, GD3 has the structure shown in FIG. 12.

In one aspect of the invention, a GD3 antibody of the invention encompasses an antibody that competes for binding to human GD3 with, and/or binds the same epitope as, an antibody, or antigen-binding fragment thereof, having the amino acid sequence of a heavy chain variable region set forth as SEQ ID NO: 1 and the amino acid sequence of a light chain variable region set forth as SEQ ID NO: 9.

In some aspects of the invention, the antibody, or antigen-binding fragment thereof, includes an IgG1 heavy chain constant region, for example a huR24 heavy chain set forth as SEQ ID NO: 1. In other aspects, the antibody, or antigen-binding fragment thereof, includes a kappa light chain constant region, for example a huR24 light chain set forth as SEQ ID NO: 9.

“huR24”, also referred to herein as “huR24vh1.1/vk1.2,” is an antibody, that specifically binds to GD3, comprising a heavy chain comprising the amino acid sequence of SEQ ID NO: 7 and a light chain comprising the amino acid sequence of SEQ ID NO: 14.

Table 1 provides the amino acid (protein) sequences and associated nucleic acid (DNA) sequences of humanized anti-GD3 antibodies of the present invention. The CDRs of huR24 VH and huR24 VL, as defined by Kabat and by Chothia, are set forth as separate sequences.

TABLE 1
Sequences of humanized anti-GD3 antibodies.

| SEQ | | |
|---|---|---|
| ID | | |
| NO | Description | Sequences |
| 1 | huR24 VH | EVQLVESGGGLVQPGGSLRLSCAASGFTFSNFGMHWVRQAPGKGLEWVAYISSGG |
| | Kabat CDRs | SSINYADTVKGRFTISRDNPKNSLYLQMNSLRAEDTAVYYCARGGTGTRSLYYFD |
| | underlined | YWGQGTLVTVSS |
| 2 | huR24 VH CDR1 | NFGMH |
| | Kabat | |
| 3 | huR24 VH CDR1 | GFTFSNF |
| | Chothia | |
| 4 | huR24 VH CDR2 | YISSGGSSINYADTVKG |
| | Kabat | |
| 5 | huR24VH CDR2 | SSGGSS |
| | Chothia | |
| 6 | huR24 VH CDR3 | GGTGTRSLYYFDY |
| | Kabat and | |
| | Chothia | |
| 7 | huR24 HC | EVQLVESGGGLVQPGGSLRLSCAASGFTFSNFGMHWVRQAPGKGLEWVAYISSGG |
| | huIgG1 | SSINYADTVKGRFTISRDNPKNSLYLQMNSLRAEDTAVYYCARGGTGTRSLYYFD |
| | Kabat CDRs | YWGQGTLVTVSSASTKGPSVFPLAPSSKSTSGGTAALGCLVKDYFPEPVTVSWNS |
| | underlined | GALTSGVHTFPAVLQSSGLYSLSSVVTVPSSSLGTQTYICNVNHKPSNTKVDKKV |
| | | EPKSCDKTHTCPPCPAPELLGGPSVFLFPPKPKDTLMISRTPEVTCVVVDVSHED |
| | | PEVKFNWYVDGVEVHNAKTKPREEQYNSTYRVVSVLTVLHQDWLNGKEYKCKVSN |
| | | KALPAPIEKTISKAKGQPREPQVYTLPPSREEMTKNQVSLTCLVKGFYPSDIAVE |
| | | WESNGQPENNYKTTPPVLDSDGSFFLYSKLTVDKSRWQQGNVFSCSVMHEALHNH |
| | | YTQKSLSLSPGK |
| 8 | huR24 HC | gaggtgcagctggtggagagcggcggcggcctggtgcagcccggcggcagcctgc |
| | DNA | ggctgagctgcgccgccagcggcttcaccttcagcaacttcggcatgcactgggt |
| | | gcggcaggcccccggcaagggcctggagtgggtggcctacatcagcagcggcggc |
| | | agcagcatcaactacgccgacaccgtgaagggccggttcaccatcagccgggaca |
| | | accccaagaacagcctgtacctgcagatgaacagcctgcgggccgaggacaccgc |
| | | cgtgtactactgcgcccggggcggcaccggcacccggagcctgtactacttcgac |
| | | tactggggccagggcaccctggtgaccgtgtcctcagcgtcgaccaagggcccat |
| | | cggtcttccccctggcaccctcctccaagagcacctctgggggcacagcggccct |
| | | gggctgcctggtcaaggactacttccccgaaccggtgacggtgtcgtggaactca |
| | | ggcgccctgaccagcggcgtgcacaccttcccggctgtcctacagtcctcaggac |
| | | tctactccctcagcagcgtggtgaccgtgccctccagcagcttgggcacccagac |
| | | ctacatctgcaacgtgaatcacaagcccagcaacaccaaggtggacaagaaagtt |
| | | gagcccaaatcttgtgacaaaactcacacatgcccaccgtgcccagcacctgaac |
| | | tcctggggggaccgtcagtcttcctcttccccccaaaacccaaggacaccctcat |
| | | gatctcccggacccctgaggtcacatgcgtggtggtggacgtgagccacgaagac |
| | | cctgaggtcaagttcaactggtacgtggacggcgtggaggtgcataatgccaaga |
| | | caaagccgcgggaggagcagtacaacagcacgtaccgtgtggtcagcgtcctcac |
| | | cgtcctgcaccaggactggctgaatggcaaggagtacaagtgcaaggtctccaac |
| | | aaagccctcccagcccccatcgagaaaaccatctccaaagccaaagggcagcccc |
| | | gagaaccacaggtgtacaccctgcccccatcccgggaggagatgaccaagaacca |
| | | ggtcagcctgacctgcctggtcaaaggcttctatcccagcgacatcgccgtggag |
| | | tgggagagcaatgggcagccggagaacaactacaagaccacgcctcccgtgctgg |
| | | actccgacggctccttcttcctctatagcaagctcaccgtggacaagagcaggtg |
| | | gcagcaggggaacgtcttctcatgctccgtgatgcatgaggctctgcacaaccac |
| | | tacacgcagaagagcctctccctgtccccgggtaaa |
| 9 | huR24 VL | DIQMTQSPSSLSASVGDRVTITCRASQDIGNFLNWYQQKPGKAPKLLIYYTSRLQ |
| | Kabat CDRs | SGVPSRFSGWGSGTDFTLTISSLQPEDFATYYCQQGKTLPYTFGGGTKVEIK |
| | underlined | |
| 10 | huR24 VL CDR1 | RASQDIGNFLN |
| | Kabat | |
| 11 | huE22 VL CDR1 | RASQDIGNFLN |
| | Chothia | |
| 12 | huR24 VL CDR2 | YTSRLQS |
| | Kabat and | |
| | Chothia | |
| 13 | huR24 VL CDR3 | QQGKTLPYT |
| | Kabat and | |
| | Chothia | |
| 14 | huR24 LC | DIQMTQSPSSLSASVGDRVTITCRASQDIGNFLNWYQQKPGKAPKLLIYYTSRLQ |
| | human Kappa | SGVPSRFSGWGSGTDFTLTISSLQPEDFATYYCQQGKTLPYTFGGGTKVEIKRTV |
| | Kabat CDRs | AAPSVFIFPPSDEQLKSGTASVVCLLNNFYPREAKVQWKVDNALQSGNSQESVTE |
| | underlined | QDSKDSTYSLSSTLTLSKADYEKHKVYACEVTHQGLSSPVTKSFNRGEC |
| 15 | huR24 LC | cgtgagtagaataactctagaggaatagggaagctaggaagaaactcaaaacatc |
| | DNA | aagattttaaatacgcttcttggtctccttgctataattatctgggataagcatg |
| | | ctgttttctgtctgtccctaacatgccctgtgattatccgcaaacaacacaccca |
| | | agggcagaactttgttacttaaacaccatcctgtttgcttctttcctcaggaact |
| | | gtggctgcaccatctgtcttcatcttcccgccatctgatgagcagttgaaatctg |
| | | gaactgcctctgttgtgtgcctgctgaataacttctatcccagagaggccaaagt |
| | | acagtggaaggtggataacgccctccaatcgggtaactcccaggagagtgtcaca |
| | | gagcaggacagcaaggacagcacctacagcctcagcagcaccctgacgctgagca |
| | | aagcagactacgagaaacacaaagtctacgcctgcgaagtcacccatcagggcct |
| | | gagctcgcccgtcacaaagagcttcaacaggggagagtgt |
| 16 | mR24 VH | DVQLVESGGGLVQPGGSRKLSCAASGFTFSNFGMHWVRQAPEKGLEWVAYISSGG |
| | Kabat CDRs | SSINYADTVKGRFTISRDNPKNTLFLQMTSLRSEDTAIYYCTRGGTGTRSLYYFD |
| | underlined | YWGQGATLIVSS |
| 17 | mR24 HC | DVQLVESGGGLVQPGGSRKLSCAASGFTFSNFGMHWVRQAPEKGLEWVAYISSGG |
| | murine IgG3 | SSINYADTVKGRFTISRDNPKNTLFLQMTSLRSEDTAIYYCTRGGTGTRSLYYFD |
| | Kabat CDRs | YWGQGATLIVSSATTTAPSVYPLVPGCSDTSGSSVTLGCLVKGYFPEPVTVKWNY |
| | underlined | GALSSGVRTVSSVLQSGFYSLSSLVTVPSSTWPSQTVICNVAHPASKTELIKRIE |
| | | PRIPKPSTPPGSSCPPGNILGGPSVFIFPPKPKDALMISLTPKVTCVVVDVSEDD |
| | | PDVHVSWFVDNKEVHTAWTQPREAQYNSTFRVVSALPIQHQDWMRGKEFKCKVNN |
| | | KALPAPIERTISKPKGRAQTPQVYTIPPPREQMSKKKVSLTCLVTNFFFEAISVE |
| | | WERNGELEQDYKNTPPILDSDGTYFLYSKLTVDTDSWLQGENFTCSVVHEALHNH |
| | | HTQKNLSRSPGK |
| 18 | mR24 VL | DIQMTQITSSLSVSLGDRVIISCRASQDIGNFLNWYQQKPDGSLKLLIYYTSRLQ |
| | Kabat CDRs | SGVPSRFSGWGSGTDYSLTISNLEEEDIATFFCQQGKTLPYTFGGGTKLEIK |
| | underlined | |
| 19 | mR24 LC | DIQMTQITSSLSVSLGDRVIISCRASQDIGNFLNWYQQKPDGSLKLLIYYTSRLQ |
| | murine Kappa | SGVPSRFSGWGSGTDYSLTISNLEEEDIATFFCQQGKTLPYTFGGGTKLEIKRAD |
| | Kabat CDRs | AAPTVSIFPPSSEQLTSGGASVVCFLNNFYPKDINVKWKIDGSERQNGVLNSWTD |
| | underlined | QDSKDSTYSMSSTLTLTKDEYERHNSYTCEATHKTSTSPIVKSFNRNEC |
| 20 | LD47 VH | QVQLVESGGGVVQPGRSLRLSCAASGFTFSNFGMHWVRQAPGKGLEWVAYISSGG |
| | Kabat CDRs | SSINYADTVKGRFTISRDNSKNTLYLQMNSLRAEDTAVYYCTRGGTGTRSLYYFD |
| | underlined | YWGQGTTVTVSS |
| 21 | LD47 HC | QVQLVESGGGVVQPGRSLRLSCAASGFTFSNFGMHWVRQAPGKGLEWVAYISSGG |
| | HuIgG1 Kabat | SSINYADTVKGRFTISRDNSKNTLYLQMNSLRAEDTAVYYCTRGGTGTRSLYYFD |
| | CDRs | YWGQGTTVTVSSASTKGPSVFPLAPSSKSTSGGTAALGCLVKDYFPEPVTVSWNS |
| | underlined | GALTSGVHTFPAVLQSSGLYSLSSVVTVPSSSLGTQTYICNVNHKPSNTKVDKKV |
| | | EPKSCDKTHTCPPCPAPELLGGPSVFLFPPKPKDTLMISRTPEVTCVVVDVSHED |
| | | PEVKFNWYVDGVEVHNAKTKPREEQYNSTYRVVSVLTVLHQDWLNGKEYKCKVSN |
| | | KALPAPIEKTISKAKGQPREPQVYTLPPSRDELTKNQVSLTCLVKGFYPSDIAVE |
| | | WESNGQPENNYKTTPPVLDSDGSFFLYSKLTVDKSRWQQGNVFSCSVMHEALHNH |
| | | YTQKSLSLSPGK |
| 22 | LD47 VL | DIQMTQSPSSLSASVGDRVTITCRASQDIGNFLNWYQQKPGKAPKLLIYYTSRLQ |
| | Kabat CDRs | SGVPSRFSGSGSGTDYTLTISSLQPEDFATYYCQQGKTLPYTFGGGTKVEIK |
| | underlined | |
| 23 | LD47 LC | DIQMTQSPSSLSASVGDRVTITCRASQDIGNFLNWYQQKPGKAPKLLIYYTSRLQ |
| | Human Kappa | SGVPSRFSGSGSGTDYTLTISSLQPEDFATYYCQQGKTLPYTFGGGTKVEIKRTV |
| | Kabat CDRs | AAPSVFIFPPSDEQLKSGTASVVCLLNNFYPREAKVQWKVDNALQSGNSQESVTE |
| | underlined | QDSKDSTYSLSSTLTLSKADYEKHKVYACEVTHQGLSSPVTKSFNRGEC |
| 24 | LD49 VH | QVQLVESGGGVVQPGRSLRLSCAASGFTFSNFGMHWVRQAPGKGLEWVAYISSGG |
| | Kabat CDRs | SSINYADTVKGRFTISRDNSKNTLYLQMNSLRAEDTAVYYCTRGGTGTRSLYYFD |
| | underlined | YWGQGITVTVSS |
| 25 | LD49 HC | QVQLVESGGGVVQPGRSLRLSCAASGFTFSNFGMHWVRQAPGKGLEWVAYISSGG |
| | HuIgG1 Kabat | SSINYADTVKGRFTISRDNSKNTLYLQMNSLRAEDTAVYYCTRGGTGTRSLYYFD |
| | CDRs | YWGQGITVTVSSASTKGPSVFPLAPSSKSTSGGTAALGCLVKDYFPEPVTVSWNS |
| | underlined | GALTSGVHTFPAVLQSSGLYSLSSVVTVPSSSLGTQTYICNVNHKPSNTKVDKKV |
| | | EPKSCDKTHTCPPCPAPELLGGPSVFLFPPKPKDTLMISRTPEVTCVVVDVSHED |
| | | PEVKFNWYVDGVEVHNAKTKPREEQYNSTYRVVSVLTVLHQDWLNGKEYKCKVSN |
| | | KALPAPIEKTISKAKGQPREPQVYTLPPSRDELTKNQVSLTCLVKGFYPSDIAVE |
| | | WESNGQPENNYKTTPPVLDSDGSFFLYSKLTVDKSRWQQGNVFSCSVMHEALHNH |
| | | YTQKSLSLSPGK |
| 26 | LD 49 VL | DIQMTQSPSSLSASVGDRVTITCRASQDIGNFLNWYQQKPGKAPKLLLYYTSRLQ |
| | Kabat CDRs | SGVPSRFSGSGSGTDYTLTISSLQPEDFATYYCQQGKTLPYTFGGGTKVEIK |
| | underlined | |
| 27 | LD 49 LC | DIQMTQSPSSLSASVGDRVTITCRASQDIGNFLNWYQQKPGKAPKLLLYYTSRLQ |
| | Kabat CDRs | SGVPSRFSGSGSGTDYTLTISSLQPEDFATYYCQQGKTLPYTFGGGTKVEIKRTV |
| | underlined | AAPSVFIFPPSDEQLKSGTASVVCLLNNFYPREAKVQWKVDNALQSGNSQESVTE |
| | | QDSKDSTYSLSSTLTLSKADYEKHKVYACEVTHQGLSSPVTKSFNRGEC |
| 28 | KM871 VH | EVTLVESGGDFVKPGGSLKVSCAASGFAFSHYAMSWVRQTPAKRLEWVAYISSGG |
| | | SGTYYSDSVKGRFTISRDNAKNTLYLQMRSLRSEDSAMYFCTRVKLGTYYFDSWG |
| | | QGTTLTVSS |
| 29 | KM871 VL | DIQMTQTASSLPASLGDRVTISCSASQDISNYLNWYQQKPDGTVKLLIFYSSNLH |
| | | SGVPSRFSGGGSGTDYSLTISNLEPEDIATYFCHQYSKLPWTFGGGTKLEIK |
| 30 | hAb 21 VH | EVQLVESGGGLVQPGGSLRLSCAASGFTFSNFGMHWVRQAPGKGLEWVAYISSGG |
| | | SSINYADTVKGRFTISRDNAKNSLYLQMNSLRAEDTAVYYCARGGTGTRSLYYFD |
| | | YWGQGTLVTVSS |
| 31 | hAb 21 VL | EIVLTQSPATLSLSPGERATLSCRASQDIGNFLNWYQQKPGQAPRLLIYYTSRLQ |
| | | SGIPARFSGSGSGTDFTLTISSLEPEDFAVYYCQQGKTLPYTFGGGTKVEIK |
| 32 | hAb 3 VH | EVQLVESGGGLVQPGGSLRLSCAASGFTFSNFGMHWVRQAPGKGLEWVAYISSGG |
| | | SSINYADTVKGRFTISRDNAKNSLYLQMNSLRAEDTAVYYCARGGTGTRSLYYFD |
| | | YWGQGTLVTVSS |
| 33 | hAb 3 VL | DIQMTQSPSSVSASVGDRVTITCRASQDIGNFLNWYQQKPGKAPKLLIYYTSRLQ |
| | | SGVPSRFSGSGSGTDFTLTISSLQPEDFATYYCQQGKTLPYTFGGGTKVEIK |
| 34 | hR24VH1.0 | EVQLVESGGGLVQPGGSLRLSCAASGFTFSNFGMHWVRQAPGKGLEWVAYISSGG |
| | Kabat CDRs | SSINYADTVKGRFTISRDNAKNSLYLQMNSLRAEDTAVYYCARGGTGTRSLYYFD |
| | underlined | YWGQGTLVTVSS |
| 35 | hR24VH1.4 | EVQLVESGGGLVQPGGSLRLSCAASGFTFSNFGMHWVRQAPGKGLEWVAYISSGG |
| | Kabat CDRs | SSINYADTVKGRFTISRDNPKNSLYLQMTSLRAEDTAVYYCARGGTGTRSLYYFD |
| | underlined | YWGQGTLVTVSS |
| 36 | hR24VL1.0 | DIQMTQSPSSLSASVGDRVTITCRASQDIGNFLNWYQQKPGKAPKLLIYYTSRLQ |
| | Kabat CDRs | SGVPSRFSGSGSGTDFTLTISSLQPEDFATYYCQQGKTLPYTFGGGTKVEIK |
| | underlined | |
| 37 | hR24 VL1.1 | DIQMTQSPSSLSASVGDRVTITCRASQDIGNFLNWYQQKPDGSLKLLIYYTSRLQS |
| | Kabat CDRs | FSGSGSGTDFTLTISSLQPEDFATYYCQQGKTLPYTFGGGTKVEIK |
| | underlined | |

### Nucleic Acids

The invention also provides polynucleotides encoding any of the antibodies of the invention, including antibody portions and modified antibodies described herein. The invention also provides a method of making any of the polynucleotides described herein. Polynucleotides can be made and expressed by procedures known in the art.

The sequence of a desired antibody, or antigen-binding fragment thereof, and nucleic acid encoding such antibody, or antigen-binding fragment thereof, can be determined using standard sequencing techniques. A nucleic acid sequence encoding a desired antibody, or antigen-binding fragment thereof, may be inserted into various vectors (such as cloning and expression vectors) for recombinant production and characterization. A nucleic acid encoding the heavy chain, or an antigen-binding fragment of the heavy chain, and a nucleic acid encoding the light chain, or an antigen-binding fragment of the light chain, can be cloned into the same vector, or different vectors.

In one aspect, the invention provides polynucleotides encoding the amino acid sequences of the GD3-binding antibody huR24.

The invention provides polynucleotides encoding one or more proteins comprising the amino acid sequence selected from the group consisting of: (i) SEQ ID NOs: 1-7 and 9-14.

The invention provides polynucleotides comprising the nucleic acid sequence as set forth as one or more of SEQ ID NOs: 7 and 14. The invention provides a polynucleotide comprising the nucleic acid sequence as set forth as SEQ ID NO: 7. The invention provides a polynucleotide comprising the nucleic acid sequence as set forth as SEQ ID NO: 14.

The invention provides a polynucleotide comprising the nucleic acid coding sequence of the DNA insert of the nucleic acid molecule deposited with the ATCC and having Accession No. PTA-124057 encoding the VH domain of huR24, and the coding sequence of the DNA insert of the nucleic acid molecule deposited with the ATCC and having Accession No. PTA-124058 encoding the VL domain of huR24. The invention provides a polynucleotide comprising the nucleic acid molecule deposited with the ATCC and having Accession No. PTA-124057. The invention provides a polynucleotide comprising the nucleic acid molecule deposited with the ATCC and having Accession No. PTA-124058.

In another aspect, the invention provides polynucleotides and variants thereof encoding an anti-GD3 antibody, wherein such variant polynucleotides share at least 70%, at least 75%, at least 80%, at least 85%, at least 87%, at least 89%, at least 90%, at least 91%, at least 92%, at least 93%, at least 94%, at least 95%, at least 96%, at least 97%, at least 98%, or at least 99% nucleic acid sequence identity to any of the specific nucleic acid sequences disclosed or referred to herein. These amounts are not meant to be limiting, and increments between the recited percentages are specifically envisioned as part of the disclosure.

The invention provides polypeptides encoded by the nucleic acid molecules described herein.

In one embodiment, the VH and VL domains, or antigen-binding portion thereof, or full-length HC or LC, are encoded by separate polynucleotides. Alternatively, both VH and VL, or antigen-binding portion thereof, or HC and LC, are encoded by a single polynucleotide.

Polynucleotides complementary to any such sequences are also encompassed by the present disclosure. Polynucleotides may be single-stranded (coding or antisense) or double-stranded, and may be DNA (genomic, cDNA or synthetic) or RNA molecules. RNA molecules include HnRNA molecules, which contain introns and correspond to a DNA molecule in a one-to-one manner, and mRNA molecules, which do not contain introns. Additional coding or non-coding sequences may, but need not, be present within a polynucleotide of the present disclosure, and a polynucleotide may, but need not, be linked to other molecules and/or support materials.

Polynucleotides may comprise a nucleic acid sequence that encodes an antibody or a portion thereof or may comprise a variant of such a sequence. Polynucleotide variants contain one or more substitutions, additions, deletions and/or insertions such that the binding characteristics of the encoded polypeptide is not diminished relative to a native antibody molecule. The effect on the binding characteristics of the polypeptide encoded by the variant nucleic acid sequence may generally be assessed as described herein. In some embodiments, polynucleotide variants exhibit at least about 70% identity, in some embodiments, at least about 80% identity, in some embodiments, at least about 90% identity, and in some embodiments, at least about 95% identity to a polynucleotide sequence that encodes the original (parent) antibody not comprising any substitution, addition, deletion and/or insertion, or a portion thereof. These percent identities are not meant to be limiting, and increments between the recited percentages are specifically envisioned as part of the disclosure.

Two polynucleotide or polypeptide sequences are said to be “identical” if the sequence of nucleotides or amino acids in the two sequences is the same when aligned for maximum correspondence as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, or 40 to about 50, in which a sequence may be compared to a reference sequence of the same number of contiguous positions after the two sequences are optimally aligned.

Optimal alignment of sequences for comparison may be conducted using the MegAlign® program in the Lasergene® suite of bioinformatics software (DNASTAR®, Inc., Madison, Wis.), using default parameters. This program embodies several alignment schemes described in the following references: Dayhoff, M. O., 1978, A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J., 1990, Unified Approach to Alignment and Phylogenes pp. 626-645 Methods in Enzymology vol. 183, Academic Press, Inc., San Diego, Calif.; Higgins, D. G. and Sharp, P. M., 1989, CABIOS 5:151-153; Myers, E. W. and Muller W., 1988, CABIOS 4:11-17; Robinson, E. D., 1971, Comb. Theor. 11:105; Santou, N., Nes, M., 1987, Mol. Biol. Evol. 4:406-425; Sneath, P. H. A. and Sokal, R. R., 1973, Numerical Taxonomy the Principles and Practice of Numerical Taxonomy, Freeman Press, San Francisco, Calif.; Wilbur, W. J. and Lipman, D. J., 1983, Proc. Natl. Acad. Sci. USA 80:726-730.

In some embodiments, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residue occurs in both sequences to yield the number of matched positions, dividing the number of matched positions by the total number of positions in the reference sequence (i.e., the window size) and multiplying the results by 100 to yield the percentage of sequence identity.

Polynucleotide variants may also, or alternatively, be substantially homologous to a gene, or a portion or complement thereof. Such polynucleotide variants are capable of hybridizing under moderately stringent conditions to a naturally occurring DNA sequence encoding an antibody (or a complementary sequence).

Suitable “moderately stringent conditions” include prewashing in a solution of 5×SSC, 0.5% SDS, 1.0 mM EDTA (pH 8.0); hybridizing at about 50° C. to 65° C., 5×SSC (0.75 M NaCl, 0.075 M sodium citrate), overnight; followed by washing twice at 65° C. for 20 minutes with each of 2×, 0.5× and 0.2×SSC containing 0.1% SDS.

As used herein, “highly stringent conditions” or “high stringency conditions” are those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC, 50 mM sodium phosphate (pH 6.8), 0.1% sodium pyrophosphate, 5×Denhardt's solution, sonicated salmon sperm DNA (50 μg/mL), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C. The skilled artisan will recognize how to adjust the temperature, ionic strength, etc. as necessary to accommodate factors such as probe length and the like.

It will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the present disclosure. Further, alleles of the genes comprising the polynucleotide sequences provided herein are within the scope of the present disclosure. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

The polynucleotides of this disclosure can be obtained using chemical synthesis, recombinant methods, or PCR. Methods of chemical polynucleotide synthesis are well known in the art and need not be described in detail herein. One of skill in the art can use the sequences provided herein and a commercial DNA synthesizer to produce a desired DNA sequence.

For preparing polynucleotides using recombinant methods, a polynucleotide comprising a desired sequence can be inserted into a suitable vector, and the vector in turn can be introduced into a suitable host cell for replication and amplification, as further discussed herein. Polynucleotides may be inserted into host cells by any means known in the art. Cells are transformed by introducing an exogenous polynucleotide by direct uptake, endocytosis, transfection, F-mating or electroporation. Once introduced, the exogenous polynucleotide can be maintained within the cell as a non-integrated vector (such as a plasmid) or integrated into the host cell genome. The polynucleotide so amplified can be isolated from the host cell by methods well known within the art. See, e.g., Sambrook et al., 1989.

Alternatively, PCR allows reproduction of DNA sequences. PCR technology is well known in the art and is described in U.S. Pat. Nos. 4,683,195, 4,800,159, 4,754,065 and 4,683,202, as well as PCR: The Polymerase Chain Reaction, Mullis et al. eds., Birkauswer Press, Boston, 1994.

RNA can be obtained by using the isolated DNA in an appropriate vector and inserting it into a suitable host cell. When the cell replicates and the DNA is transcribed into RNA, the RNA can then be isolated using methods well known to those of skill in the art, as set forth in Sambrook et al., 1989, for example.

Suitable cloning and expression vectors can include a variety of components, such as promoter, enhancer, and other transcriptional regulatory sequences. The vector may also be constructed to allow for subsequent cloning of an antibody variable domain into different vectors. Suitable cloning vectors may be constructed according to standard techniques, or may be selected from a large number of cloning vectors available in the art. While the cloning vector selected may vary according to the host cell intended to be used, useful cloning vectors will generally have the ability to self-replicate, may possess a single target for a particular restriction endonuclease, and/or may carry genes for a marker that can be used in selecting clones containing the vector. Suitable examples include plasmids and bacterial viruses, e.g., pUC18, pUC19, Bluescript (e.g., pBS SK+) and its derivatives, mp18, mp19, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen. Expression vectors are further provided. Expression vectors generally are replicable polynucleotide constructs that contain a polynucleotide according to the disclosure. It is implied that an expression vector must be replicable in the host cells either as episomes or as an integral part of the chromosomal DNA. Suitable expression vectors include but are not limited to plasmids, viral vectors, including adenoviruses, adeno-associated viruses, retroviruses, cosmids, and expression vector(s) disclosed in PCT Publication No. WO 87/04462. Vector components may generally include, but are not limited to, one or more of the following: a signal sequence; an origin of replication; one or more marker genes; suitable transcriptional controlling elements (such as promoters, enhancers and terminator). For expression (i.e., translation), one or more translational controlling elements are also usually required, such as ribosome binding sites, translation initiation sites, and stop codons.

The vectors containing the polynucleotides of interest and/or the polynucleotides themselves, can be introduced into a host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell.

The antibody, or antigen-binding fragment thereof, may be made recombinantly using a suitable host cell. A nucleic acid encoding the antibody or antigen-binding fragment thereof can be cloned into an expression vector, which can then be introduced into a host cell, such as E. coli cell, a yeast cell, an insect cell, a simian COS cell, a Chinese hamster ovary (CHO) cell, or a myeloma cell where the cell does not otherwise produce an immunoglobulin protein, to obtain the synthesis of an antibody in the recombinant host cell. Preferred host cells include a CHO cell, a Human embryonic kidney (HEK) 293 cell, or a Sp2.0 cell, among many cells well-known in the art. An antibody fragment can be produced by proteolytic or other degradation of a full-length antibody, by recombinant methods, or by chemical synthesis. A polypeptide fragment of an antibody, especially shorter polypeptides up to about 50 amino acids, can be conveniently made by chemical synthesis. Methods of chemical synthesis for proteins and peptides are known in the art and are commercially available.

### Antibody Drug Conjugates

Anti-GD3 ADCs of the present invention can be prepared using a linker to link or conjugate a drug to an anti-GD3 antibody. Such conjugates allow the selective delivery of cytotoxic drugs to tumor cells.

“Antibody-drug conjugate” or “ADC” refers to antibodies, or antigen-binding fragments thereof, including antibody derivatives that bind to GD3 and are conjugated to a drug such as a cytotoxic, cytostatic, and/or therapeutic agent, as described further herein below. For example, a cytotoxic agent can be linked or conjugated to an anti-GD3 antibody as described herein for targeted local delivery of the cytotoxic agent to tumors (e.g., GD3 expressing tumor).

For preparation of GD3 ADCs of the invention, the antibody, or antigen-binding fragment thereof, can be any anti-GD3 antibody, or antigen-binding fragment thereof, described herein. The antibody, or antigen-binding fragment thereof, may be isolated, purified, or derivatized for use in preparation of GD3 ADCs.

For use in preparation of ADCs, the GD3 antibodies described herein may be substantially pure, i.e., at least 50% pure (i.e., free from contaminants), more preferably, at least 90% pure, more preferably, at least 95% pure, yet more preferably, at least 98% pure, and most preferably, at least 99% pure.

The present invention provides ADCs of the formula Ab-(L-D)p, wherein (a) Ab is an antibody, or antigen-binding fragment thereof, that binds to GD3, (b) L-D is a linker-drug moiety, wherein L is a linker, and D is a drug, and (c) p represents the drug-to-antibody ratio (DAR) or average drug loading, indicating the number of drug molecules conjugated per antibody. Also provided are methods of preparing and manufacturing such ADCs, and use of the same in clinical applications.

In particular aspects of the invention, a GD3 ADC of the formula Ab-(L-D)p includes (a) an antibody (Ab), or antigen-binding fragment thereof, including a heavy chain variable region set forth as SEQ ID NO: 1 and a light chain variable region set forth as SEQ ID NO: 9; (b) a linker-drug moiety (L-D), wherein L is a linker, and D is a drug, wherein the linker is mcValCitPABC, and wherein the drug is auristatin 0101, (2-methylalanyl-N-[(3R,4S,5S)-3-methoxy-1-{(2S)-2-[(1R,2R)-1-methoxy-2-methyl-3-oxo-3-{[(1 S)-2-phenyl-1-(1,3-thiazol-2-yl)ethyl]amino}propyl]pyrrolidin-1-yl}-5-methyl-1-oxoheptan-4-yl]-N-methyl-L-valinamide); and (c) p is an integer from about 1 to about 12, where p is also referred to as the “drug-to-antibody ratio” (DAR).

In another aspect of the invention, a GD3 ADC of the formula Ab-(L-D)p includes (a) an antibody (Ab), or antigen-binding fragment thereof, including a heavy chain variable region set forth as SEQ ID NO: 1 and a light chain variable region set forth as SEQ ID NO: 9; (b) a linker-drug moiety (L-D), wherein L is a linker, and D is a drug, wherein the linker is mcValCitPABC, and wherein the drug is auristatin 0101, (2-methylalanyl-N-[(3R,4S,5S)-3-methoxy-1-{(2S)-2-[(1R,2R)-1-methoxy-2-methyl-3-oxo-3-{[(1 S)-2-phenyl-1-(1,3-thiazol-2-yl)ethyl]amino}propyl]pyrrolidin-1-yl}-5-methyl-1-oxoheptan-4-yl]-N-methyl-L-valinamide); and (c) p is 4 (i.e., DAR is 4).

In another aspect of the invention, a GD3 ADC of the formula Ab-(L-D)p includes (a) an antibody (Ab), or antigen-binding fragment thereof, including a heavy chain set forth as SEQ ID NO: 7 and a light chain set forth as SEQ ID NO: 14; (b) a linker-drug moiety (L-D), wherein L is a linker, and D is a drug, wherein the linker is mcValCitPABC, and wherein the drug is auristatin 0101, (2-methylalanyl-N-[(3R,4S,5S)-3-methoxy-1-{(2S)-2-[(1R,2R)-1-methoxy-2-methyl-3-oxo-3-{[(1 S)-2-phenyl-1-(1,3-thiazol-2-yl)ethyl]amino}propyl]pyrrolidin-1-yl}-5-methyl-1-oxoheptan-4-yl]-N-methyl-L-valinamide); and (c) p is 4 (DAR).

“huR24-ADC” is an ADC of the formula, Ab-(L-D)p, wherein: (a) Ab is the antibody huR24 vh1.1/vk1.2, (b) L is the linker mcValCitPABC, and (c) D is the drug auristatin 0101 (Aur101), and (d) p is 4.

### Linkers

U.S. Pat. No. 8,828,401, which is incorporated herein by reference in its entirety, discloses linkers that may be used with an anti-GD3 antibody.

In one aspect, a second section of the linker unit is introduced which has a second reactive site e.g., an electrophilic group that is reactive to a nucleophilic group present on an antibody unit (e.g., an antibody). Useful nucleophilic groups on an antibody include but are not limited to, sulfhydryl, hydroxyl and amino groups. The heteroatom of the nucleophilic group of an antibody is reactive to an electrophilic group on a linker unit and forms a covalent bond to a linker unit. Useful electrophilic groups include, but are not limited to, maleimide and haloacetamide groups. The electrophilic group provides a convenient site for antibody attachment.

In another embodiment, a linker unit has a reactive site which has a nucleophilic group that is reactive to an electrophilic group present on an antibody. Useful electrophilic groups on an antibody include, but are not limited to, aldehyde and ketone carbonyl groups. The heteroatom of a nucleophilic group of a linker unit can react with an electrophilic group on an antibody and form a covalent bond to the antibody. Useful nucleophilic groups on a linker unit include, but are not limited to, hydrazide, oxime, amino, hydrazine, thiosemicarbazone, hydrazine carboxylate, and arylhydrazide. The electrophilic group on an antibody provides a convenient site for attachment to a linker unit.

Amino functional groups are also useful reactive sites for a linker unit because they can react with carboxylic acid, or activated esters of a compound to form an amide linkage. Typically, the peptide-based compounds of the invention can be prepared by forming a peptide bond between two or more amino acids and/or peptide fragments. Such peptide bonds can be prepared, for example, according to the liquid phase synthesis method (see, e.g., Schroder and Lubke, “The Peptides”, volume 1, pp 76-136, 1965, Academic Press) that is well known in the field of peptide chemistry.

As described in more detail below, the conjugates can be prepared using a section of the linker having a reactive site for binding to a compound of the invention and introducing another section of the linker unit having a reactive site for an antibody. In one aspect, a linker unit has a reactive site which has an electrophilic group that is reactive with a nucleophilic group present on an antibody unit, such as an antibody. The electrophilic group provides a convenient site for antibody attachment. Useful nucleophilic groups on an antibody include but are not limited to, sulfhydryl, hydroxyl and amino groups. The heteroatom of the nucleophilic group of an antibody is reactive to an electrophilic group on a Linker unit and forms a covalent bond to a linker unit. Useful electrophilic groups include, but are not limited to, maleimide and haloacetamide groups.

In another embodiment, a linker unit has a reactive site which has a nucleophilic group that is reactive with an electrophilic group present on an antibody unit. The electrophilic group on an antibody provides a convenient site for attachment to a linker unit. Useful electrophilic groups on an antibody include, but are not limited to, aldehyde and ketone carbonyl groups. The heteroatom of a nucleophilic group of a linker unit can react with an electrophilic group on an antibody and form a covalent bond to the antibody. Useful nucleophilic groups on a linker unit include, but are not limited to, hydrazide, oxime, amino, hydrazine, thiosemicarbazone, hydrazine carboxylate, and arylhydrazide.

As used herein, “mcValCitPABC-” also known as “MalCValCitPABC-” refers to

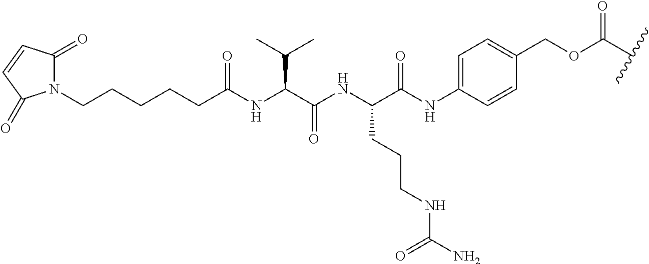 [SMILES: *C(=O)OCc1ccc(NC(=O)[C@H](CCCNC(N)=O)NC(=O)[C@@H](NC(=O)CCCCCN2C(=O)C=CC2=O)C(C)C)cc1]

The linker molecule may be stable (non-cleavable) or hydrolysable (cleavable) whereby it is released from the antibody following cellular entry of the ADC. The major mechanisms by which the linker-drug is cleaved from the antibody include hydrolysis of the cleavable linker in the acidic pH of the lysosomes (e.g., hydrazones, acetals, and cis-aconitate-like amides, among others), peptide cleavage by lysosomal enzymes (including, but not limited to, the cathepsins and other lysosomal enzymes), and reduction of disulfide bonds. As a result of these varying mechanisms for cleavage, mechanisms of linking the drug to the antibody also vary widely and any suitable linker can be selected as would be understood in the art.

An example of a suitable conjugation procedure relies on the conjugation of hydrazides and other nucleophiles to the aldehydes generated by oxidation of the carbohydrates that naturally occur on antibodies. Hydrazone-containing conjugates can be made with introduced carbonyl groups that provide the desired drug-release properties. Conjugates can also be made with a linker that has a disulfide at one end, an alkyl chain in the middle, and a hydrazine derivative at the other end. The anthracyclines are one example of cytotoxins that can be conjugated to antibodies using this technology.

Linkers containing functional groups other than hydrazones have the potential to be cleaved in the acidic milieu of the lysosomes. For example, conjugates can be made from thiol-reactive linkers that contain a site other than a hydrazone that is cleavable intracellularly, such as esters, amides, and acetals/ketals. Camptothecin is one cytotoxic agent that can be conjugated using these linkers. Ketals made from a 5 to 7-member ring ketone and that has one of the oxygens attached to the cytotoxic agent and the other to a linker for antibody attachment also can be used. The anthracyclines are also an example of a suitable cytotoxin for use with these linkers.

Another example of a class of pH sensitive linkers are the cis-aconitates, which have a carboxylic acid juxtaposed to an amide bond. The carboxylic acid accelerates amide hydrolysis in the acidic lysosomes. Linkers that achieve a similar type of hydrolysis rate acceleration with several other types of structures can also be used. The maytansinoids are an example of a cytotoxin that can be conjugated with linkers attached at C-9.

Another potential release method for drug conjugates is the enzymatic hydrolysis of peptides by the lysosomal enzymes. In one example, a peptide is attached via an amide bond to para-aminobenzyl alcohol and then a carbamate or carbonate is made between the benzyl alcohol and the cytotoxic agent. Cleavage of the peptide leads to the collapse, or self-immolation, of the aminobenzyl carbamate or carbonate. The cytotoxic agents exemplified with this strategy include anthracyclines, taxanes, mitomycin C, and the auristatins. In one example, a phenol can also be released by collapse of the linker instead of the carbamate. In another variation, disulfide reduction is used to initiate the collapse of a para-mercaptobenzyl carbamate or carbonate.

Many of the cytotoxic agents conjugated to antibodies have little, if any, solubility in water and that can limit drug loading on the conjugate due to aggregation of the conjugate. One approach to overcoming this is to add solubilizing groups to the linker. Conjugates made with a linker consisting of PEG and a dipeptide can been used, including those having a PEG di-acid, thiol-acid, or maleimide-acid attached to the antibody, a dipeptide spacer, and an amide bond to the amine of an anthracycline or a duocarmycin analogue. Another example is a conjugate prepared with a PEG-containing linker disulfide bonded to a cytotoxic agent and amide bonded to an antibody. Approaches that incorporate PEG groups may be beneficial in overcoming aggregation and limits in drug loading.

In some aspects of the invention, the linkers for the preparation of the ADCs of the present invention include linkers having the formula:

(CO-Alk₁-Sp₁-Ar-Sp²-Alk²-C(Z¹)=Q-Sp)

wherein
Alk¹ and Alk² are independently a bond or branched or unbranched (C₁-C₁₀) alkylene chain;
Sp¹ is a bond, —S—, —O—, —CONH—, —NHCO—, —NR′—, —N(CH₂CH₂)₂N—, or —X—Ar′—Y—(CH₂)ₙ—Z wherein X, Y, and Z are independently a bond, —NR′—, —S—, or —O—, with the proviso that when n=0, then at least one of Y and Z must be a bond and Ar′ is 1,2-, 1,3-, or 1,4-phenylene optionally substituted with one, two, or three groups of (C₁-C₅) alkyl, (C₁-C₄) alkoxy, (C₁-C₄) thioalkoxy, halogen, nitro, —COOR′, —CONHR′, —(CH₂)ₙCOOR′, —S(CH₂)ₙCOOR′, —O(CH₂)ₙCONHR′, or —S(CH₂)ₙCONHR′, with the proviso that when Alk′ is a bond, Sp¹ is a bond;
n is an integer from 0 to 5;
R′ is a branched or unbranched (C₁-C₅) chain optionally substituted by one or two groups of —OH, (C₁-C₄) alkoxy, (C₁-C₄) thioalkoxy, halogen, nitro, (C₁-C₃) dialkylamino, or (C₁-C₃) trialkylammonium -A⁻ where A⁻ is a pharmaceutically acceptable anion completing a salt;
Ar is 1,2-, 1,3-, or 1,4-phenylene optionally substituted with one, two, or three groups of (C₁-C₆) alkyl, (C₁-C₅) alkoxy, (C₁-C₄) thioalkoxy, halogen, nitro, —COOR′, —CONHR′, —O(CH₂)ₙCOOR′, —S(CH₂)ₙCOOR′, —O(CH₂)ₙCONHR′, or —S(CH₂)ₙCONHR′ wherein n and R′ are as hereinbefore defined or a 1,2-, 1,3-, 1,4-, 1,5-, 1,6-, 1,7-, 1,8-, 2,3-, 2,6-, or 2,7-naphthylidene or

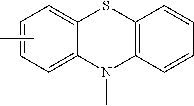

with each naphthylidene or phenothiazine optionally substituted with one, two, three, or four groups of (C₁-C₆) alkyl, (C₁-C₅) alkoxy, (C₁-C₄) thioalkoxy, halogen, nitro, —COOR′, —CONHR′, —O(CH₂)ₙCOOR′, —S(CH₂)ₙCOOR′, or —S(CH₂)ₙCONHR′ wherein n and R′ are as defined above, with the proviso that when Ar is phenothiazine, Sp¹ is a bond only connected to nitrogen;
Sp² is a bond, —S—, or —O—, with the proviso that when Alk² is a bond, Sp² is a bond, Z¹ is H, (C₁-C₅) alkyl, or phenyl optionally substituted with one, two, or three groups of (C₁-C₅) alkyl, (C₁-C₅) alkoxy, (C₁-C₄) thioalkoxy, halogen, nitro, —COOR′, —ONHR′, —O(CH₂)ₙCOOR′, —S(CH₂)ₙCOOR′, —O(CH₂)ₙCONHR′, or —S(CH₂)ₙCONHR′ wherein n and R′ are as defined above;
Sp is a straight or branched-chain divalent or trivalent (C₁-C₁₈) radical, divalent or trivalent aryl or heteroaryl radical, divalent or trivalent (C₃-C₁₈) cycloalkyl or heterocycloalkyl radical, divalent or trivalent aryl- or heteroaryl-aryl (C₁-C₁₈) radical, divalent or trivalent cycloalkyl- or heterocycloalkyl-alkyl (C₁-C₁₈) radical or divalent or trivalent (C₂-C₁₈) unsaturated alkyl radical, wherein heteroaryl is preferably furyl, thienyl, N-methylpyrrolyl, pyridinyl, N-methylimidazolyl, oxazolyl, pyrimidinyl, quinolyl, isoquinolyl, N-methylcarbazoyl, aminocourmarinyl, or phenazinyl and wherein if Sp is a trivalent radical, Sp may be additionally substituted by lower (C₁-C₅) dialkylamino, lower (C₁-C₅) alkoxy, hydroxy, or lower (C₁-C₅) alkylthio groups; and
Q is ═NHNCO—, ═NHNCS—, ═NHNCONH—, ═NHNCSNH—, or ═NHO—.

Preferably, Alk¹ is a branched or unbranched (C₁-C₁₀) alkylene chain; Sp′ is a bond, —S—, —O—, —CONH—, —NHCO—, or —NR′ wherein R′ is as hereinbefore defined, with the proviso that when Alk′ is a bond, Sp¹ is a bond;
Ar is 1,2-, 1,3-, or 1,4-phenylene optionally substituted with one, two, or three groups of (C₁-C₆) alkyl, (C₁-C₅) alkoxy, (C₁-C₄) thioalkoxy, halogen, nitro, —COOR′, —CONHR′, —O(CH₂)ₙCOOR′, —S(CH₂)ₙCOOR′, —O(CH₂)ₙCONHR′, or —S(CH₂)ₙCONHR′ wherein n and R′ are as hereinbefore defined, or Ar is a 1,2-, 1,3-, 1,4-, 1,5-, 1,6-, 1,7-, 1,8-, 2,3-, 2,6-, or 2,7-naphthylidene each optionally substituted with one, two, three, or four groups of (C₁-C₆) alkyl, (C₁-C₅) alkoxy, (C₁-C₄) thioalkoxy, halogen, nitro, —COOR′, —CONHR′, —O(CH₂)ₙCOOR′, —S(CH₂)ₙCOOR′, —O(CH₂)ₙCONHR′, or —S(CH₂)ₙCONHR′.
Z¹ is (C₁-C₅) alkyl, or phenyl optionally substituted with one, two, or three groups of (C₁-C₅) alkyl, (C₁-C₄) alkoxy, (C₁-C₄) thioalkoxy, halogen, nitro, —COOR′, —CONHR′, —O(CH₂)ₙCOOR′, —S(CH₂)ₙCOOR′, —O(CH₂)ₙCONHR′, or —S(CH₂)ₙCONHR′; Alk² and Sp² are together a bond; and Sp and Q are as immediately defined above.

U.S. Pat. No. 5,773,001, which is incorporated herein by reference in its entirety, discloses linkers that may be used with nucleophilic drugs, particularly hydrazides and related nucleophiles, prepared from the calicheamicins. These linkers are especially useful in those cases where better activity is obtained when the linkage formed between the drug and the linker is hydrolysable. These linkers contain two functional groups, including (1) a group for reaction with an antibody (e.g., carboxylic acid), and (2) a carbonyl group (e.g., an aldehyde or a ketone) for reaction with a drug. The carbonyl groups may react with a hydrazide group on the drug to form a hydrazone linkage. This linkage is cleavable hydrolysable, allowing for release of the therapeutic agent from the conjugate after binding to the target cells. In some aspects of the invention, the hydrolysable linker used is 4-(4-acetylphenoxy) butanoic acid (AcBut). In other aspects of the invention, ADCs can be prepared using (3-Acetylphenyl) acetic acid (AcPAc) or 4-mercapto-4-methyl-pentanoic acid (Amide) as the linker molecule.

N-hydroxysuccinimide (OSu) esters or other comparably activated esters can be used to generate the activated hydrolyzable linker-drug moiety. Examples of other suitable activating esters include NHS (N-hydroxysuccinimide), sulfo-NHS (sulfonated NHS), PFP (pentafluorophenyl), TFP (tetrafluorophenyl), and DNP (dinitrophenyl).

In some aspects of the invention, the ADCs are prepared by reacting calicheamicin or derivatives thereof, the 4-(4-acetylphenoxy) butanoic acid linker and an anti-GD3 antibody of the present invention. See e.g., U.S. Pat. No. 5,773,001. The 4-(4-acetylphenoxy) butanoic acid linker produces conjugates that are substantially stable in circulation, releasing an estimated 2% of the calicheamicin per day when assayed at 37° C. in human plasma in vitro. The conjugates release the calicheamicin in the acidic lysosomes.

In some aspects of the invention, the 4-(4-acetylphenoxy) butanoic acid-calicheamicin moiety can be generated using methods and processes described in the art, such as PCT International Publication No. WO 08/147765 and in U.S. Pat. No. 8,273,862, which are incorporated herein by reference in their entirety.

In some aspects of the invention, the 4-(4-acetylphenoxy) butanoic acid-calicheamicin moiety can be generated using an improved synthesis process, as described in U.S. Application Publication No. 2016/0251389, which is incorporated herein by reference in its entirety.

The invention includes linkers where the linker can be a dipeptide linker, such as a valine-citrulline (val-cit), a phenylalanine-lysine (phe-lys) linker, or maleimidocapronic-valine-citruline-p-aminobenzyloxycarbonyl (vc) linker. In another aspect, the linker is Sulfosuccinimidyl-4-[N-maleimidomethyl]cyclohexane-1-carboxylate (smcc). Sulfo-smcc conjugation occurs via a maleimide group which reacts with sulfhydryls (thiols, —SH), while its Sulfo-NHS ester is reactive toward primary amines (as found in Lysine and the protein or peptide N-terminus). Further, the linker may be maleimidocaproyl (mc).

Representative linkers useful for conjugation of radioisotopes include diethylenetriamine pentaacetate (DTPA)-isothiocyanate, succinimidyl 6-hydrazinium nicotinate hydrochloride (SHNH), and hexamethylpropylene amine oxime (HMPAO) (Bakker et al. (1990) J. Nucl. Med. 31: 1501-1509, Chattopadhyay et al. (2001) Nucl. Med. Biol. 28: 741-744, Dewanjee et al. (1994) J. Nucl. Med. 35: 1054-63, Krenning et al. (1989) Lancet 1: 242-244, Sagiuchi et al. (2001) Ann. Nucl. Med. 15: 267-270); U.S. Pat. No. 6,024,938). Alternatively, a targeting molecule may be derivatized so that a radioisotope may be bound directly to it (Yoo et al. (1997) J. Nucl. Med. 38: 294-300). Iodination methods are also known in the art, and representative protocols may be found, for example, in Krenning et al. (1989) Lancet 1:242-4 and in Bakker et al. (1990) J. Nucl. Med. 31:1501-9.

In particular aspects of the invention, the linker of the GD3 ADCs of the invention includes, but is not limited to, mcValCitPABC.

### Drugs

Drugs useful in preparation of the disclosed GD3 ADCs include any substance having biological activity, for example, therapeutic agents, detectable labels, binding agents, etc., and prodrugs, which are metabolized to an active agent in vivo. A drug may also be a drug derivative, wherein a drug has been functionalized to enable conjugation with an antibody of the invention. In accordance with the disclosed methods, the drugs are used to prepare an ADCs of the formula Ab-(L-D)p, wherein (a) Ab is an antibody, or antigen-binding fragment thereof, that binds to GD3, (b) L-D is a linker-drug moiety, wherein L is a linker, and D is a drug, and (c) p is an integer that specifies the drug-to-antibody ratio (DAR), also referred to as the average drug loading, indicating the number of drug molecules conjugated per antibody. The average drug loading indicates the overall average number of drug moieties per antibody in a heterogeneous ADC population. That is, an average drug loading of 4 indicates that some antibody molecules may have fewer than 4 drug moieties per antibody molecule and others may have more than 4 drug moieties per antibody molecule but the overall average number of drug moieties per antibody molecule for the population is about 4 drug moieties per antibody molecule. As noted previously, “p” is an integer within the range of 1 to about 12 and specifies the DAR for an ADC. Thus, in aspects of the invention, a GD3 ADC may have a DAR of 1, a DAR of 2, a DAR of 3, a DAR of 4, a DAR of 5, a DAR of 6, a DAR of 7, a DAR of 8, a DAR of 9, a DAR of 10, a DAR of 11, a DAR of 12 or a DAR greater than 12. In aspects of the invention, a GD3 ADC may have one drug molecule, or 2 drug molecules, or 3 drug molecules, or 4 drug molecules, or 5 drug molecules, or 6 drug molecules, or 7 drug molecules, or 8 drug molecules, or 9 drug molecules, or 10 drug molecules, or 11 drug molecules, or 12 drug molecules or greater than 12 molecules conjugated per each antibody molecule.

The term “drug-to-antibody ratio” or “DAR” refers to the number of drugs, e.g., auristatin, attached to the antibody of the ADC. The DAR of an ADC can range from 1 to 12, although higher loads, e.g., 16, are also possible depending on the number of linkage site on an antibody. The term DAR may be used in reference to the number of drug molecules loaded onto an individual antibody, or, alternatively, may be used in reference to the average or mean DAR of a group of ADCs to reflect average drug loading.

Compositions, batches, and/or formulations of a plurality of ADCs may be characterized by an average DAR. DAR and average DAR can be determined by various conventional means such as UV spectroscopy, mass spectroscopy, ELISA assay, radiometric methods, hydrophobic interaction chromatography (HIC), electrophoresis and HPLC.

In one embodiment, a therapeutic agent (e.g., a drug molecule or moiety) is an agent that exerts a cytotoxic, cytostatic, and/or immunomodulatory effect on a cell, including a cancer cell or a cell exhibiting abnormal growth characteristics compared with an otherwise identical but not abnormal cell. Abnormal growth characteristic can include, but is not limited to, a decreased time of cell cycle growth or division, greater growth in size, and loss of contact inhibition such that the cell can grow to a greater density compared with an otherwise identical but normal cell. Examples of therapeutic agents that can be conjugated to an antibody of the invention include cytotoxic agents, chemotherapeutic agents, cytostatic agents, and immunomodulatory agents, among others. These agents are useful in the treatment of cancer.

Therapeutic agents are compositions that may be used to treat or prevent a condition in a subject in need thereof. Therapeutic agents useful in the invention include anti-cancer agents, i.e., agents having anti-cancer activity a cell such as a cancer cell from cancers including, but not limited to melanoma, breast cancer, glioma, glioblastoma, and lung cancer, wherein the cancer cell expresses GD3.

Representative therapeutic agents include cytotoxins, cytotoxic agents, and cytostatic agents. A cytotoxic effect refers to the depletion, elimination and/or the killing of a target cell(s). A cytotoxic agent refers to an agent that has a cytotoxic and/or cytostatic effect on a cell. A cytostatic effect refers to the inhibition of cell growth and/or proliferation. Therefore, a cytostatic agent refers to an agent that has a cytostatic effect on a cell, thereby inhibiting the growth and/or expansion of a cell.

Additional representative therapeutic agents include radioisotopes, anti-angiogenic agents, anti-proliferative agents, pro-apoptotic agents, and cytolytic enzymes (e.g., RNAses). An agent may also include a therapeutic nucleic acid, such as a gene encoding an immunomodulatory agent, an anti-angiogenic agent, an anti-proliferative agent, or a pro-apoptotic agent. These drug descriptors are not mutually exclusive, and thus a therapeutic agent may be described using one or more of the above-noted terms. For example, selected radioisotopes are also cytotoxins. Therapeutic agents may be prepared as pharmaceutically acceptable salts, acids or derivatives of any of the above. Generally, conjugates having a radioisotope as the drug are referred to as radioimmunoconjugates and those having a chemotherapeutic agent as the drug are referred to as chemoimmunoconjugates.

Examples of cytotoxic agents include, but are not limited to an anthracycline, an auristatin, CC-1065, a dolastatin, a duocarmycin, an enediyne, a geldanamycin, a maytansine, a puromycin, a taxane, a vinca alkaloid, SN-38, tubulysin, hemiasterlin, and stereoisomers, isosteres, analogs or derivatives thereof. Plant toxins, other bioactive proteins, enzymes (i.e., ADEPT), radioisotopes, photosensitizers (i.e., for photodynamic therapy) can also be used.

The anthracyclines are derived from bacteria Streptomyces and have been used to treat a wide range of cancers, such as leukemias, lymphomas, breast, uterine, ovarian, and lung cancers. Exemplary anthracyclines include, but are not limited to, daunorubicin, doxorubicin (i.e., adriamycin), epirubicin, idarubicin, valrubicin, and mitoxantrone.

Dolastatins and their peptidic analogs and derivatives, auristatins, are highly potent antimitotic agents that have been shown to have anticancer and antifungal activity. See, e.g., U.S. Pat. No. 5,663,149 and Pettit et al., Antimicrob. Agents Chemother. 42:2961-2965, (1998). Exemplary dolastatins and auristatins include, but are not limited to, dolastatin 10, auristatin E, auristatin EB (AEB), auristatin EFP (AEFP), MMAD (Monomethyl Auristatin D or monomethyl dolastatin 10), MMAF (Monomethyl Auristatin F or N-methylvaline-valine-dolaisoleuine-dolaproine-phenylalanine), MMAE (Monomethyl Auristatin E or N-methylvaline-valine-dolaisoleuine-dolaproine-norephedrine), 5-benzoylvaleric acid-AE ester (AEVB).

In some aspects of the invention, auristatins described in PCT International Publication No. WO 2013/072813, which is incorporated herein by reference in its entirety, and methods of producing those auristatins are used herein.

For example, the auristatin 0101, (2-methylalanyl-N-[(3R,4S,5S)-3-methoxy-1-{(2S)-2-[(1R,2R)-1-methoxy-2-methyl-3-oxo-3-{[(1 S)-2-phenyl-1-(1,3-thiazol-2-yl)ethyl]amino}propyl]pyrrolidin-1-yl}-5-methyl-1-oxoheptan-4-yl]-N-methyl-L-valinamide), having the following structure:

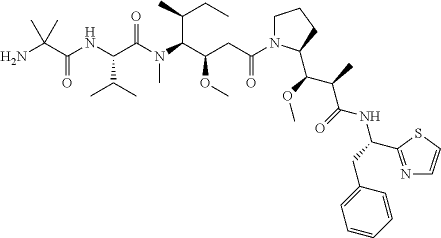 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)C(C)(C)N)C(C)C]

Additionally, the auristatin 8261, 2-Methylalanyl-N-[(3R,4S,5S)-1-{(2S)-2-[(1R,2R)-3-{[(1S)-1-carboxy-2-phenylethyl]amino}-1-methoxy-2-methyl-3-oxopropyl]pyrrolidin-1-yl}-3-methoxy-5-methyl-1-oxoheptan-4-yl]-N-methyl-L-valinamide, having the following structure:

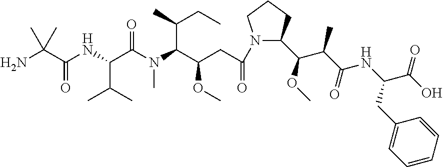 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)C(C)(C)N)C(C)C]

Duocarmycin and CC-1065 are DNA alkylating agents with cytotoxic potency. See Boger and Johnson, PNAS 92:3642-3649, 1995. Exemplary dolastatins and auristatins include, but are not limited to, (+)-docarmycin A and (+)-duocarmycin SA, and (+)-CC-1065.

Enediynes are a class of anti-tumor bacterial products characterized by either nine- and ten-membered rings or the presence of a cyclic system of conjugated triple-double-triple bonds. Exemplary enediynes include, but are not limited to, calicheamicin, esperamicin, and dynemicin.

In some aspects of the invention, the cytotoxic agent is an antibiotic, such as calicheamicin, also called the LL-E33288 complex, for example, β-calicheamicin, γ-calicheamicin or N-acetyl-γ-calicheamicin (gamma-calicheamicin (γ₁)). Examples of calicheamicins suitable for use in the present invention are disclosed, for example, in U.S. Pat. Nos. 4,671,958, 4,970,198, 5,053,394, 5,037,651, 5,079,233 and 5,108,912, which are incorporated herein by reference in its entirety. These compounds contain a methyltrisulfide that may be reacted with appropriate thiols to form disulfides, at the same time introducing a functional group such as a hydrazide or other functional group that is useful for conjugating calicheamicin to an GD3 antibody. Disulfide analogs of calicheamicin can also be used, for example, analogs described in U.S. Pat. Nos. 5,606,040 and 5,770,710, which are incorporated herein by reference in its entirety. In some aspects of the invention, the disulfide analog is N-acetyl-γ-calicheamicin dimethyl hydrazide (hereinafter “CM”).

Geldanamycins are benzoquinone ansamycin antibiotic that bind to Hsp90 (Heat Shock Protein 90) and have been used antitumor drugs. Exemplary geldanamycins include, but are not limited to, 17-AAG (17-N-Allylamino-17-Demethoxygeldanamycin) and 17-DMAG (17-Dimethylaminoethylamino-17-demethoxygeldanamycin).

Maytansines or their derivatives maytansinoids inhibit cell proliferation by inhibiting the microtubules formation during mitosis through inhibition of polymerization of tubulin. See Remillard et al., Science 189:1002-1005, 1975. Exemplary maytansines and maytansinoids include, but are not limited to, mertansine (DM1) and its derivatives as well as ansamitocin.

Taxanes are diterpenes that act as anti-tubulin agents or mitotic inhibitors. Exemplary taxanes include, but are not limited to, paclitaxel (e.g., TAXOL®) and docetaxel (TAXOTERE®).

Vinca alkyloids are also anti-tubulin agents. Exemplary vinca alkyloids include, but are not limited to, vincristine, vinblastine, vindesine, and vinorelbine.

In some aspects of the invention, the drug is an immunomodulating agent. Examples of an immunomodulating agent include, but are not limited to, ganciclovir, etanercept, tacrolimus, sirolimus, voclosporin, cyclosporine, rapamycin, cyclophosphamide, azathioprine, mycophenolgate mofetil, methotrextrate, glucocorticoid and its analogs, cytokines, xanthines, stem cell growth factors, lymphotoxins, tumor necrosis factor (TNF), hematopoietic factors, interleukins (e.g., interleukin-1 (IL-1), IL-2, IL-3, IL-6, IL-10, IL-12, IL-18, and IL-21), colony stimulating factors (e.g., granulocyte-colony stimulating factor (G-CSF) and granulocyte macrophage-colony stimulating factor (GM-CSF)), interferons (e.g., interferons-α, -β and -γ), the stem cell growth factor designated “S 1 factor,” erythropoietin and thrombopoietin, or a combination thereof.

Immunomodulatory agents useful in the invention also include anti-hormones that block hormone action on tumors and immunosuppressive agents that suppress cytokine production, down-regulate self-antigen expression, or mask MHC antigens. Representative anti-hormones include anti-estrogens including, for example, tamoxifen, raloxifene, aromatase inhibiting 4(5)-imidazoles, 4-hydroxytamoxifen, trioxifene, keoxifene, LY 117018, onapnstone, and toremifene; and anti-androgens such as flutamide, nilutamide, bicalutamide, leuprolide, and goserelin; and anti-adrenal agents. Representative immunosuppressive agents include 2-amino-6-aryl-5-substituted pyrimidines, azathioprine, cyclophosphamide, bromocryptine, danazol, dapsone, glutaraldehyde, anti-idiotypic antibodies for MHC antigens and MHC fragments, cyclosporin A, steroids such as glucocorticosteroids, cytokine or cytokine receptor antagonists (e.g., anti-interferon antibodies, anti-IL10 antibodies, anti-TNFα antibodies, anti-IL2 antibodies), streptokinase, TGFβ, rapamycin, T-cell receptor, T-cell receptor fragments, and T cell receptor antibodies.

In some aspects of the invention, the drug is a therapeutic protein including, but is not limited to, a toxin, a hormone, an enzyme, and a growth factor.

Examples of a toxin protein (or polypeptide) include, but are not limited to, dipththeria toxin (e.g., diphtheria A chain), Pseudomonas exotoxin and endotoxin, ricin (e.g., ricin A chain), abrin (e.g., abrin A chain), modeccin (e.g., modeccin A chain), alpha-sarcin, Aleurites fordii proteins, dianthin proteins, ribonuclease (RNase), DNase I, Staphylococcal enterotoxin-A, pokeweed antiviral protein, gelonin, diphtherin toxin, Phytolaca americana proteins (PAPI, PAPII, and PAP-S), momordica charantia inhibitor, curcin, crotin, sapaonaria officinalis inhibitor, mitogellin, restrictocin, phenomycin, enomycin, tricothecenes, inhibitor cystine knot (ICK) peptides (e.g., ceratotoxins), and conotoxin (e.g., KIIIA or SmIIIa).

Examples of hormones include, but are not limited to, estrogens, androgens, progestins and corticosteroids.

In some aspects of the invention, the cytotoxic agent can be made using a liposome or biocompatible polymer. The anti-GD3 antibodies as described herein can be conjugated to the biocompatible polymer to increase serum half-life and bioactivity, and/or to extend in vivo half-lives. Examples of biocompatible polymers include water-soluble polymer, such as polyethylene glycol (PEG) or its derivatives thereof and zwitterion-containing biocompatible polymers (e.g., a phosphorylcholine containing polymer).

In some aspects of the invention, the drug is an oligonucleotide, such as anti-sense oligonucleotides.

Additional drugs useful in the invention include anti-angiogenic agents that inhibit blood vessel formation, for example, farnesyltransferase inhibitors, COX-2 inhibitors, VEGF inhibitors, bFGF inhibitors, steroid sulphatase inhibitors (e.g., 2-methoxyoestradiol bis-sulphamate (2-MeOE2bisMATE)), interleu kin-24, thrombospondin, metallospondin proteins, class I interferons, interleukin 12, protamine, angiostatin, laminin, endostatin, and prolactin fragments.

Anti-proliferative agents and pro-apoptotic agents include activators of PPAR-gamma (e.g., cyclopentenone prostaglandins (cyPGs)), retinoids, triterpinoids (e.g., cycloartane, lupane, ursane, oleanane, friedelane, dammarane, cucurbitacin, and limonoid triterpenoids), inhibitors of EGF receptor (e.g., HER4), rampamycin, CALCITRIOL® (1,25-dihydroxycholecalciferol (vitamin D)), aromatase inhibitors (FEMARA® (letrozone)), telomerase inhibitors, iron chelators (e.g., 3-aminopyridine-2-carboxaldehyde thiosemicarbazone (Triapine)), apoptin (viral protein 3-VP3 from chicken aneamia virus), inhibitors of Bcl-2 and Bcl-X(L), TNF-alpha, FAS ligand, TNF-related apoptosis-inducing ligand (TRAIL/Apo2L), activators of TNF-alpha/FAS ligand/TNF-related apoptosis-inducing ligand (TRAIL/Apo2L) signaling, and inhibitors of PI3K-Akt survival pathway signaling (e.g., UCN-01 and geldanamycin).

Representative chemotherapeutic agents include alkylating agents such as thiotepa and cyclophosphamide; alkyl sulfonates such as busulfan, improsulfan and piposulfan; aziidines such as benzodopa, carboquone, meturedopa, and uredopa; ethylenimines and methylamelamines including altretamine, triethylenemelamine, triethylenephosphoramide, triethylenethiophosphoramide and trimethylolomelamine; nitrogen mustards such as chlorambucil, chlornaphazine, cholophosphamide, estramustine, ifosfamide, mechiorethamine, mechiorethamine oxide hydrochloride, melphalan, novembichin, phenesterine, prednimustine, trofosfarnide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-EU; androgens such as calusterone, dromostanolone propionate, epitiostanol, mepitiostane, testolactone; anti-adrenal such as arninoglutethimide, mitotane, trilostane; folic acid replenisher such as frolinic acid; aceglatone; aldophospharnide glycoside; arninolevulinic acid; amsacrine; bestrabucil; bisantrene; edatraxate; defofamine; demecolcine; diaziquone; elfornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; razoxane; sizofiran; spirogermanium; tenuazonic acid; triaziquone; 2,2′,2′-trichlorotriethylamine; urethan; vindesine; dacarbazine; mannomustine; mitobronitol; mitolactol; pipobroman; gacytosine; arabinoside (Ara-C); cyclophosphamide; thiotepa; taxoids, e.g. paclitaxel (TAXOL®, Bristol-Myers Squibb Oncology of Princeton, N.J.) and doxetaxel (TAXOTERE®, Rhone-Poulenc Rorer of Antony, France); chiorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aininopterin; xeloda; ibandronate; CPT-11; topoisomerase inhibitor RFS 2000; difluoromethylornithine (DMFO); retinoic acid; esperamicins; and capecitabine.

Additional therapeutic agents that may be used in accordance with the present invention include photosensitizing agents, such as U.S. Publication No. 20020197262 and U.S. Pat. No. 5,952,329, which are incorporated herein by reference in its entirety, for photodynamic therapy; magnetic particles for thermotherapy, such as U.S. Publication No. 20030032995, which is incorporated herein by reference in its entirety; binding agents, such as peptides, ligands, cell adhesion ligands, etc., and prodrugs such as phosphate-containing prodrugs, thiophosphate-containing prodrugs, sulfate containing prodrugs, peptide containing prodrugs, β-lactam-containing prodrugs, substituted phenoxyacetamide-containing prodrugs or substituted phenylacetamide-containing prodrugs, 5-fluorocytosine and other 5-fluorouridine prodrugs that may be converted to the more active cytotoxic free drug.

### Diagnostic Methods

For diagnostic methods using anti-GD3 antibodies, the conjugated drug may include a detectable label used to detect the presence of GD3-expressing cells in vitro or in vivo. Radioisotopes that are detectable in vivo, such as those labels that are detectable using scintigraphy, magnetic resonance imaging, or ultrasound, may be used in clinical diagnostic applications. Useful scintigraphic labels include positron emitters and γ-emitters. Representative contrast agents for magnetic source imaging are paramagnetic or superparamagnetic ions (e.g., iron, copper, manganese, chromium, erbium, europium, dysprosium, holmium and gadolinium), iron oxide particles, and water-soluble contrast agents. For ultrasonic detection, gases or liquids may be entrapped in porous inorganic particles that are released as microbubble contrast agents. For in vitro detection, useful detectable labels include fluorophores, detectable epitopes or binding agents, and radioactive labels.

Thus, in some aspects of the invention, the drug is an imaging agent (e.g., a fluorophore or a PET (Positron Emission Tomography) label, SPECT (Single-Photon Emission Computed Tomorgraphy) label), or MRI (Magnetic Resonance Imaging) label.

In Vivo Detection and Diagnosis

In another aspect, provided is a method of detecting, diagnosing, and/or monitoring a condition associated with GD3 expression. For example, the GD3 antibodies or ADCs as described herein can be labeled with a detectable moiety such as an imaging agent and an enzyme-substrate label. The GD3 antibodies or ADCs as described herein can also be used for in vivo diagnostic assays, such as in vivo imaging (e.g., PET or SPECT), or a staining reagent.

Following administration of a GD3 antibody or ADC to a subject, wherein the drug is a detectable label, and after a time sufficient for binding, the biodistribution of GD3-expressing cells bound by the antibody or ADC may be visualized. The disclosed diagnostic methods may be used in combination with treatment methods. In addition, GD3 antibody or ADCs of the invention may be administered for the dual purpose of detection and therapy.

Representative non-invasive detection methods include scintigraphy (e.g., SPECT (Single Photon Emission Computed Tomography), PET (Positron Emission Tomography), gamma camera imaging, and rectilinear scanning), magnetic resonance imaging (e.g., convention magnetic resonance imaging, magnetization transfer imaging (MTI), proton magnetic resonance spectroscopy (MRS), diffusion-weighted imaging (DWI) and functional MR imaging (fMRI)), and ultrasound.

The term “label” when used herein refers to a detectable compound or composition that is conjugated directly or indirectly to the antibody so as to generate a “labeled” antibody. The label may be detectable by itself (e.g., radioisotope labels or fluorescent labels) or, in the case of an enzymatic label, may catalyze chemical alteration of a substrate compound or composition that is detectable. Radionuclides that can serve as detectable labels include, for example, I-131, I-123, I-125, Y-90, Re-188, Re-186, At-211, Cu-67, Bi-212, and Pd-109. The label might also be a non-detectable entity such as a toxin.

Examples of fluorophores include, but are not limited to, fluorescein isothiocyanate (FITC) (e.g., 5-FITC), fluorescein amidite (FAM) (e.g., 5-FAM), eosin, carboxyfluorescein, erythrosine, Alexa Fluor® (e.g., Alexa 350, 405, 430, 488, 500, 514, 532, 546, 555, 568, 594, 610, 633, 647, 660, 680, 700, or 750), carboxytetramethylrhodamine (TAMRA) (e.g., 5,-TAMRA), tetramethylrhodamine (TMR), and sulforhodamine (SR) (e.g., SR101).

Therapeutic or diagnostic radioisotopes or other labels (e.g., PET or SPECT labels) can be incorporated in the agent for conjugation to the anti-GD3 antibodies as described herein. The isotope may be directly bound to the antibody, for example, at a cysteine residue present in the antibody, or a chelator may be used to mediate the binding of the antibody and the radioisotope. Radioisotopes suitable for radiotherapy include but are not limited to α-emitters, β-emitters, and auger electrons. For diagnostic applications, useful radioisotopes include positron emitters and γ-emitters. An anti-GD3 antibody of the invention may further be iodinated, for example, on a tyrosine residue of the antibody, to facilitate detection or therapeutic effect of the antibody.

Examples of a radioisotope or other labels include, but are not limited to, ³H, ¹¹C, ¹³N, ¹⁴C, ¹⁵N, ¹⁵O, ³⁵S, ¹⁸F, ³²P, ³³P, ⁴⁷Sc, ⁵¹Cr, ⁵⁷Co, ⁵⁸Co, ⁵⁹Fe, ⁶²Cu, ⁶⁴Cu, ⁶⁷Cu, ⁶⁷Ga, ⁶⁸Ga, ⁷⁵Se, ⁷⁶Br, ⁷⁷Br, ⁸⁶Y, ⁸⁹Zr, ⁹⁰Y, ⁹⁴Tc, ⁹⁵Ru, ⁹⁷Ru, ⁹⁹Tc, ¹⁰³Ru, ¹⁰⁵Rh, ¹⁰⁵Ru, ¹⁰⁷Hg, ¹⁰⁹Pd, ¹¹¹Ag, ¹¹¹In, ¹¹³In, ¹²¹Te, ¹²²Te, ¹²³I, ¹²⁴I, ¹²⁵I, ¹²⁵Te, ¹²⁶I, ¹³¹I, ¹³¹In, ¹³³I, ¹⁴²Pr, ¹⁴³Pr, ¹⁵³Pb, ¹⁵³Sm, ¹⁶¹Tb, ¹⁶⁵Tm, ¹⁶⁶Dy, ¹⁶⁶H, ¹⁶⁷Tm, ¹⁶⁸Tm, ¹⁶⁹Yb, ¹⁷⁷Lu, ¹⁸⁶Re, ¹⁸⁸Re, ¹⁸⁹Re, ¹⁹⁷Pt, ¹⁹⁸Au, ¹⁹⁹Au, ²⁰¹Tl, ²⁰³Hg, ²¹¹At, ²¹²Bi, ²¹²Pb, ²¹³Bi, ²²³Ra, ²²⁴Ac, and ²²⁵Ac.

### Methods of Preparing GD3 Antibody-Drug Conjugates

Also provided are methods for preparing the ADCs of the present invention. For example, a process for producing a GD3 ADC as disclosed herein can include (a) linking the linker to the drug moiety; (b) conjugating the linker-drug moiety to the antibody; and (c) purifying the ADC.

In some aspects, GD3 ADCs may be generated using conventional, non-specific conjugation of linker-payload moieties through one or more cysteine residues of an anti-GD3 antibody, or an antigen binding fragment thereof.

In another aspect, GD3 ADCs may be generated using site-specific conjugation of linker-payload moieties though one or more reactive cysteine residues engineered into an anti-GD3 antibody constant domain. Methods of preparing antibodies for site-specific conjugation via engineered cysteine residues are described in PCT International Publication No. WO2013/093809, which is incorporated herein by reference in its entirety.

Optimal reaction conditions for formation of a conjugate may be empirically determined by variation of reaction variables such as temperature, pH, linker-calicheamicin moiety input, and additive concentration. Conditions suitable for conjugation of other drugs may be determined by those skilled in the art without undue experimentation. A representative method for conjugating and characterizing GD3 ADCs is described in Example 6.

Following conjugation, the conjugates may be separated, purified from unconjugated reactants and/or aggregated forms of the conjugates, and characterized by conventional methods. This includes processes such as, but not limited to, mass spectrometry, size exclusion chromatography (SEC), ultrafiltration/diafiltration, ion exchange chromatography (IEC), chromatofocusing (CF), site-directed mutagenesis, fluorescence-labeling, X-ray crystallography, high performance liquid chromatography (HPLC), fast protein liquid chromatography (FPLC), Sephacryl S-200 chromatography or hydrophobic interaction chromatography (HIC). Suitable HIC media includes, but is not limited to, Phenyl Sepharose 6 Fast Flow chromatographic medium, Butyl Sepharose 4 Fast Flow chromatographic medium, Octyl Sepharose 4 Fast Flow chromatographic medium, Toyopearl Ether-650M chromatographic medium, Macro-Prep methyl HIC medium or Macro-Prep t-Butyl HIC medium.

### Functional Assays for Characterization of GD3 Antibodies or Antibody-Drug Conjugates

The present invention further discloses in vitro and in vivo assays to characterize the activity of a GD3 antibody or ADC, including GD3 binding activity, cellular internalization following binding to GD3 antigen presented on a cell surface, and targeting to GD3-expressing cells in a tissue or subject. In some aspects of the invention, GD3 ADCs are characterized by the neutralizing or depleting aspects of the GD3 antibody, or antigen-binding fragment thereof. In some aspects of the invention, GD3 ADCs are characterized by unexpected efficacy of a particular drug as compared to lack of efficacy of an alternate drug. In some aspects of the invention, GD3 antibodies or ADCs are characterized as outperforming a standard-of-care therapeutic agent having a same mode of action as the drug but which is not conjugated to the antibody of the invention.

Functional assays include methods for assessing the anti-cancer activity of GD3 antibodies or ADCs (e.g., the ability to destroy existing cancer cells, or to delay or prevent growth of cancer cells). Cancers targeted by ADCs of the invention include both primary and metastasized tumors and carcinomas of any tissue in a subject, including carcinomas and hematopoietic malignancies such as leukemias and lymphomas, wherein the tumor cell expresses GD3.

GD3 antibodies or ADCs having growth inhibitory activity can eliminate GD3-expressing cells or to prevent or reduce proliferation of GD3-expressing cells. Representative methods for rapid in vitro assessment of cell growth inhibition are described in Jones et al. (2001) J. Immunol. Methods 254:85-98.

GD3 antibodies or ADCs may also include an ability to induce cell death, for example, programmed cell death characterized by nuclear DNA degradation, nuclear degeneration and condensation, loss of membrane integrity, and phagocytosis. Representative assays to assess cell death are described in Hoves et al. (2003) Methods 31:127-34; Peng et al. (2002) Chin. Med. Sci. J. 17:17-21; Yasuhara et al. (2003) J. Histochem. Cytochem. 51:873-885.

For example, to assess the cytotoxicity of anti-GD3 naked antibody or GD3-ADC in vitro, GD3-expressing cancer cells and otherwise identical control cells that also express GD3 are cultured in the presence of GD3 ADCs and, separately, under identical conditions but in the absence of the GD3 antibody or GD3-ADC. The cytotoxicity of the GD3 antibody or GD3-ADC is reported as ED50 (ng/ml), which is the amount of drug given as conjugate, or as free antibody or as free drug, that causes 50% reduction of a cell culture relative to an untreated control. The number of cells in culture is determined using a vital dye (MTS) following drug exposure among other art-recognized methods.

To assess the cytotoxicity of GD3 antibodies or ADCs in vivo, tumors are prepared in immune compromised mice by subcutaneous injection of various cancer cells. GD3 antibodies or ADCs and control compounds may be administered to tumor-bearing mice, for example, by intraperitoneal injection twice a week for two weeks (q4d×4). Measurable therapeutic outcomes include inhibition of tumor cell growth.

It is understood that the present invention encompasses inhibiting or killing any tumor or cancer cell expressing GD3.

### Uses and Medical Therapies

### In Vitro Applications

The present invention provides in vitro methods using GD3 antibodies or ADCs. For example, the disclosed GD3 antibodies or ADCs may be used, either alone or in combination with cytotoxic agents or other drugs to specifically bind GD3-positive cancer cells to deplete such cells from a cell sample. Methods are also provided for inducing apoptosis and/or inhibition of cell proliferation via contacting GD3-expressing cells with a GD3 antibody or ADC. Representative in vitro methods are described herein above under the heading of “Functional Assays for Characterization of GD3 antibody-drug conjugates.”

GD3 antibodies or ADCs of the invention also have utility in the detection of GD3-positive cells in vitro based on their ability to specifically bind GD3 antigen. A method for detecting GD3-expressing cells may include: (a) preparing a biological sample having cells; (b) contacting a GD3 antibody or ADCs with the biological sample in vitro, wherein the drug is a detectable label; and (c) detecting binding the GD3 antibodies or ADCs.

### Therapeutic Applications

GD3 associated diseases or conditions include, but are not limited to melanoma, breast cancer, glioma, glioblastoma, and lung cancer. That is, GD3 is not typically expressed at high levels in normal adult tissues, such that high levels of expression of GD3 in a cell type that does not typically express GD3 is an indication that the cell is associated with or causes a disease, disorder or condition associated with or mediated by GD3 expression. As stated previously herein, diseases, disorder or conditions known to be associated with or mediated by GD3 expression include, e.g., melanoma, glioma, glioblastoma, breast cancer, and lung cancer. However, the invention encompasses any disease, disorder, or condition associated with or mediated by expression of GD3 by a cell that normally does not express GD3, and such expression can be easily detected using any method described herein or known in the art to assess the expression of GD3 in a cell or tissue.

The phrase “effective amount”, “effective dosage” or as used herein refers to an amount of a drug, compound or pharmaceutical composition necessary to achieve any one or more beneficial or desired therapeutic results. For prophylactic use, beneficial or desired results include eliminating or reducing the risk, lessening the severity, or delaying the outset of the disease, including biochemical, histological and/or behavioral symptoms of the disease, its complications and intermediate pathological phenotypes presenting during development of the disease. For therapeutic use, beneficial or desired results include clinical results such as reducing incidence or amelioration of one or more symptoms of various diseases, disorders of conditions associated with or mediated by GD3 expression, including decreasing the dose of other medications required to treat the disease, enhancing the effect of another medication, and/or delaying the progression of the disease, disorder or condition in a subject, including a patient.

In one aspect, the invention provides a method for treating a condition associated with or mediated by GD3 expression in a cell in a subject in need thereof. The invention also provides a GD3 antibody or ADC, or a pharmaceutical composition, as described herein, for use in a method for treating a disease, disorder or condition associated with or mediated by GD3 expression in a subject in need thereof. The invention further provides the use of an ADC, or a pharmaceutical composition, as described herein, in the manufacture of a medicament for treating a condition associated with or mediated by GD3 expression.

In some aspects of the invention, the method of treating a condition associated with or mediated by GD3 expression in a subject in need thereof includes administering to the subject an effective amount of a composition (e.g., pharmaceutical composition) comprising the GD3 antibody or ADCs described herein. The disease, disorder or condition associated with or mediated by GD3 expression include, but are not limited to, a proliferative disorder (e.g., cancer), including, but not limited to, melanoma, glioma, glioblastoma, breast cancer, and lung cancer.

Cancers suitable for targeting using anti-GD3 antibodies or ADCs include GD3-expressing primary and metastatic cancers, and any neoplastic disorder associated with or mediated by GD3 expression in a cell that otherwise does not express GD3.

In some aspects of the invention, provided is a method of inhibiting tumor growth or progression in a subject who has a GD3 expressing tumor, including administering to the subject in need thereof an effective amount of a composition having the GD3 antibody or ADCs as described herein. In other aspects, the invention provides a method of inhibiting metastasis of GD3 expressing cancer cells in a subject in need thereof, including administering to the subject an effective amount of a composition having the GD3 antibody or ADCs as described herein.

In other aspects, the invention provides a method of inducing regression of a GD3 expressing tumor in a subject in need thereof, including administering to the subject an effective amount of a composition comprising the GD3 antibody or ADCs of the invention.

In other aspects, the invention provides an ADC, or a pharmaceutical composition comprising the ADC, as described herein, for use in a method as described above. In other aspects, the invention provides the use of a GD3 antibody or ADC, or a pharmaceutical composition the same, of the invention, in the manufacture of a medicament for use in the methods described above.

Thus, subjects to be treated with GD3 antibodies or ADCs of the invention may be selected based on biomarker expression, including but not limited to mRNA (qPCR) of bulk tumor samples to detect elevated expression of GD3. Such screening can result in a patient population selected for enriched target (i.e., GD3) expression rather than tumor origin or histology. GD3 expression can be measured as a function of the number of cells staining for GD3 combined with the intensity of the cells staining for GD3. For example, classification of high expression of GD3 includes those patients with greater than 30% (i.e., 40%, 50% or 60%) of the cells tested by immunohistochemical staining positive for GD3 at a level of 3+(on a scale of 1 to 4), while moderate expression of the GD3 can include those patients with greater than 20% of the cell cells staining at a staining intensity level of about 1+ to 2+.

Cancer growth or abnormal proliferation refers to any one of a number of indices that suggest change within cells to a more developed cancer form or disease state. Inhibition of growth of cancer cells or cells of a non-neoplastic proliferative disorder may be assayed by methods known in the art, such as delayed or decreased tumor growth (e.g., tumor volume) and inhibition of metastasis. Other indices for measuring inhibition of cancer growth include a decrease in cancer cell survival, a decrease in number of tumor cells, decrease in tumor volume or morphology (for example, as determined using computed tomographic (CT), sonography, or other imaging method), destruction of tumor vasculature, improved performance in delayed hypersensitivity skin test, an increase in the activity of cytolytic T-lymphocytes, and a decrease in levels of tumor-specific antigens.

Desired outcomes of the disclosed therapeutic methods are generally quantifiable measures as compared to a control or baseline measurement. As used herein, relative terms such as “improve,” “increase,” or “reduce” indicate values relative to a control, such as a measurement in the same individual prior to initiation of treatment described herein, or a measurement in a control individual (or multiple control individuals) in the absence of the treatment described herein. A representative control individual is an individual afflicted with the same form of hyperproliferative disorder as the individual being treated, who is about the same age as the individual being treated (to ensure that the stages of the disease in the treated individual and the control individual are comparable.

Changes or improvements in response to therapy are generally statistically significant. As used herein, the term “significance” or “significant” relates to a statistical analysis of the probability that there is a non-random association between two or more entities. To determine whether or not a relationship is “significant” or has “significance,” statistical manipulations of the data can be “p-value.” Those p-values that fall below a user-defined cut-off point are regarded as significant. A p-value less than or equal to 0.1, less than 0.05, less than 0.01, less than 0.005, or less than 0.001 may be regarded as significant.

### Combination Therapies

In some aspects of the invention, the methods described herein further include a step of treating a subject with an additional form of therapy. In some aspects, the additional form of therapy is an additional anti-cancer therapy including, but not limited to, chemotherapy, radiation, surgery, hormone therapy, and/or additional immunotherapy.

The disclosed GD3 antibodies or ADCs may be administered as an initial treatment, or for treatment of conditions that are unresponsive to conventional therapies. In addition, the GD3 antibodies or ADCs may be used in combination with other therapies (e.g., surgical excision, radiation, additional anti-cancer drugs etc.) to thereby elicit additive or potentiated therapeutic effects and/or reduce hepatocytotoxicity of some anti-cancer agents. GD3 antibodies or ADCs of the invention may be co-administered or co-formulated with additional agents, or formulated for consecutive administration with additional agents in any order.

Representative agents useful for combination therapy include any of the drugs described herein above as useful for preparation of GD3 ADCs under the subheading “Drugs.” GD3 antibodies or ADCs of the invention may also be used in combination with other therapeutic antibodies and ADCs, including anti-GD3 antibodies other than the disclosed anti-GD3 antibodies, as well as antibodies and conjugates targeting a different antigen. Representative antibodies, which may be used alone or as an ADC, include anti-5T4 antibodies (e.g., A1, A2, and A3), anti-CD19 antibodies, anti-CD20 antibodies (e.g., RITUXAN®, ZEVALIN®, BEXXAR®), anti-CD22 antibodies, anti-CD33 antibodies (e.g., MYLOTARG®), anti-CD33 ADCs, anti-Lewis Y antibodies (e.g., Hu3S193, Mthu3S193, AGmthu3S193), anti-HER-2 antibodies (e.g., HERCEPTIN® (trastuzumab), MDX-210, OMNITARG® (pertuzumab, rhuMAb 2C4)), anti-CD52 antibodies (e.g., CAMPATH®), anti-EGFR antibodies (e.g., ERBITUX® (cetuximab), ABX-EGF (panitumumab)), anti-VEGF antibodies (e.g., AVASTIN® (bevacizumab)), anti-DNA/histone complex antibodies (e.g., ch-TNT-1/b), anti-CEA antibodies (e.g., CEA-Cide, YMB-1003) hLM609, anti-CD47 antibodies (e.g., 6H9), anti-VEGFR2 (or kinase insert domain-containing receptor, KDR) antibodies (e.g., IMC-1C11), anti-Ep-CAM antibodies (e.g., ING-1), anti-FAP antibodies (e.g., sibrotuzumab), anti-DR4 antibodies (e.g., TRAIL-R), anti-progesterone receptor antibodies (e.g., 2C5), anti-CA19.9 antibodies (e.g., GIVAREX®) and anti-fibrin antibodies (e.g., MH-1).

The disclosed GD3 antibodies or ADCs may also be administered together with one or more combinations of cytotoxic agents as part of a treatment regimen. Useful cytotoxic preparations for this purpose include CHOPP (cyclophosphamide, doxorubicin, vincristine, prednisone and procarbazine); CHOP (cyclophosphamide, doxorubicin, vincristine, and prednisone); COP (cyclophosphamide, vincristine, prednisone); CAP-BOP (cyclophosphamide, doxorubicin, procarbazine, bleomycin, vincristine and prednisone); m-BACOD (methotrexate, bleomycin, doxorubicin, cyclophosphamide, vincristine, dexamethasone, and leucovorin; ProMACE-MOPP (prednisone, methotrexate, doxorubicin, cyclophosphamide, etoposide, leukovorin, mechloethamine, vincristine, prednisone and procarbazine); ProMACE-CytaBOM (prednisone, methotrexate, doxorubicin, cyclophosphamide, etoposide, leukovorin, cytarabine, bleomycin and vincristine); MACOP-B (methotrexate, doxorubicin, cyclophosphamide, vincristine, prednisone, bleomycin and leukovorin); MOPP (mechloethamine, vincristine, prednisone and procarbazine); ABVD (adriamycin/doxorubicin, bleomycin, vinblastine and dacarbazine); MOPP (mechloethamine, vincristine, prednisone and procarbazine) alternating with ABV (adriamycin/doxorubicin, bleomycin, vinblastine); MOPP (mechloethamine, vincristine, prednisone and procarbazine) alternating with ABVD (adriamycin/doxorubicin, bleomycin, vinblastine and dacarbazine); ChlVPP (chlorambucil, vinblastine, procarbazine, prednisone); IMVP-16 (ifosfamide, methotrexate, etoposide); MIME (methyl-gag, ifosfamide, methotrexate, etoposide); DHAP (dexamethasone, high-dose cytaribine and cisplatin); ESHAP (etoposide, methylpredisolone, HD cytarabine, and cisplatin); CEPP(B) (cyclophosphamide, etoposide, procarbazine, prednisone and bleomycin); CAMP (lomustine, mitoxantrone, cytarabine and prednisone); and CVP-1 (cyclophosphamide, vincristine and prednisone); DHAP (cisplatin, high-dose cytarabine and dexamethasone); CAP (cyclophosphamide, doxorubicin, cisplatin); PV (cisplatin, vinblastine or vindesine); CE (carboplatin, etoposide); EP (etoposide, cisplatin); MVP (mitomycin, vinblastine or vindesine, cisplatin); PFL (cisplatin, 5-fluorouracil, leucovorin); IM (ifosfamide, mitomycin); IE (ifosfamide, etoposide); IP (ifosfamide, cisplatin); MIP (mitomycin, ifosfamide, cisplatin); ICE (ifosfamide, carboplatin, etoposide); PIE (cisplatin, ifosfamide, etoposide); Viorelbine and cisplatin; Carboplatin and paclitaxel; CAV (cyclophosphamide, doxorubicin, vincristine); CAE (cyclophosphamide, doxorubicin, etoposide); CAVE (cyclophosphamide, doxorubicin, vincristine, etoposide); EP (etoposide, cisplatin); and CMCcV (cyclophosphamide, methotrexate, lomustine, vincristine).

GD3 antibodies or ADCs may be used in combination with systemic anti-cancer drugs, such as epithilones (BMS-247550, Epo-906), reformulations of taxanes (Abraxane, Xyotax), microtubulin inhibitors (MST-997, TTI-237), or with targeted cytotoxins such as CMD-193 and SGN-15. Additional useful anti-cancer agents include TAXOTERE®, TARCEVA®, GEMZAR® (gemcitabine), 5-FU, AVASTIN® ERBITUX®, TROVAX®, anatumomab mafenatox, letrazole, docetaxel, and anthracyclines.

For combination therapies, a GD3 antibody or ADC and/or one or more additional therapeutic or diagnostic agents are administered within any time frame suitable for performance of the intended therapy or diagnosis. Thus, the single agents may be administered substantially simultaneously (i.e., as a single formulation or within minutes or hours) or consecutively in any order. For example, single agent treatments may be administered within about 1 year of each other, such as within about 10, 8, 6, 4, or 2 months, or within 4, 3, 2 or 1 week(s), or within about 5, 4, 3, 2 or 1 day(s). The administration of a GD3 antibody or ADC in combination with a second therapeutic agent preferably elicits a greater effect than administration of either alone.

In some aspects of the invention, more than one GD3 antibody or GD3 ADC may be present. At least one, at least two, at least three, at least four, at least five different or more GD3 antibodies or GD3 ADCs can be present. Generally, those GD3 antibodies or GD3 ADCs may have complementary activities that do not adversely affect each other. For example, one or more of the following GD3 antibody may be used: a first GD3 antibody directed to one epitope on GD3 and a second GD3 antibody directed to a different epitope on GD3.

The disclosed combination therapies may elicit a synergistic therapeutic effect, i.e., an effect greater than the sum of their individual effects or therapeutic outcomes. Measurable therapeutic outcomes are described herein. For example, a synergistic therapeutic effect may be an effect of at least about two-fold greater than the therapeutic effect elicited by a single agent, or the sum of the therapeutic effects elicited by the single agents of a given combination, or at least about five-fold greater, or at least about ten-fold greater, or at least about twenty-fold greater, or at least about fifty-fold greater, or at least about one hundred-fold greater. A synergistic therapeutic effect may also be observed as an increase in therapeutic effect of at least about 10% compared to the therapeutic effect elicited by a single agent, or the sum of the therapeutic effects elicited by the single agents of a given combination, or at least about 20%, or at least about 30%, or at least about 40%, or at least about 50%, or at least about 60%, or at least about 70%, or at least about 80%, or at least about 90%, or at least about 100%, or more. A synergistic effect is also an effect that permits reduced dosing of therapeutic agents when they are used in combination.

### Formulations

The GD3 antibodies or ADCs the invention can be formulated as a pharmaceutical composition. The pharmaceutical composition may further comprise a pharmaceutically acceptable carrier, excipient, and/or stabilizer (Remington: The Science and practice of Pharmacy 21st Ed., 2005, Lippincott Williams and Wilkins, Ed. K. E. Hoover), in the form of lyophilized formulation or aqueous solution. Acceptable carriers, excipients, or stabilizers are nontoxic to recipients at the dosages and concentrations, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). Pharmaceutically acceptable excipients are further described herein.

### Internalization

Internalization of a GD3 antibody or ADC by GD3-expressing cells may be assayed by observing the amount of antibodies or conjugates bound to the surface of the GD3-expressing cells over time. Selected GD3 ligands or their isoforms may be present in a soluble form, and at least some GD3 likely remains associated with the cell surface thereby allowing for internalization of the antibodies disclosed herein. Accordingly, anti-GD3 antibody, or ADCs of the present invention may be internalized by cells that express GD3.

Internalization of GD3 antibodies or ADCs may be assessed using a functional assay in which cells are incubated with the GD3 antibody, or ADC, and a secondary antibody Fab fragment that is conjugated to the saporin toxin. Cell viability is then measured by any suitable method, with cellular cytotoxicity indicative of antibody internalization.

Alternatively, the internalization GD3 antibodies or ADCs may be assessed using imaging technology to quantitatively measure internalization. The fluorescence signal emitted by the antibodies or ADCs in subcompartments of a cell is measured, as described in Example 5 below. This technology allows for the measurement of internalization and co-localization of the GD3 antibodies or ADCs to the endosomal and lysosomal compartments of the cell. To quantitate co-localization between internalized the GD3 antibodies or ADCs and the lysosome, samples may be incubated with the GD3 antibodies or ADCs described herein, stained with the lysosomal marker LAMP-1, and then acquired using an Amnis imaging flow cytometer. Amnis IDEAS software's “similarity” algorithm may be applied to measure the degree of spatial co-localization of the fluorescent signals from the GD3 antibodies or ADCs described herein and anti-LAMP-1 (lysosomal marker) antibodies.

The internalization measurement, expressed as the Similarity Score, is the ratio of fluorescence intensity inside the cell to the fluorescence intensity of the entire cell and is defined as the log transformed Pearson's Correlation Coefficient. This score measures the degree to which two images are linearly correlated on a pixel by pixel basis within a region of the cell. The ratio is mapped on the log scale to increase the range between the minimum and maximum values. A Similarity Score of 0 indicates no internalization because the fluorescence outside of the cell is equal to the fluorescence inside of the cell. A Similarity Score of 1 or above indicates complete internalization. In some aspects, the Similarity Score is measured using a lysosomal marker to quantitate the co-localization of the anti-GD3 antibodies described herein to the lysosomal compartments of the cell. In some aspects, the lysosomal marker is LAMP-1. In some aspects, the lysosomal marker is detected with anti-LAMP-1 antibodies.

In contrast, surprisingly, the antibodies and ADCs of the present invention demonstrate the ability to internalize to a high degree. HuR24 showed a high internalization score of about 0.8 (Example 5).

Even more surprisingly, huR24-ADC consistently demonstrated the ability to internalize and remain in the cell to an even higher degree than huR24, as evidenced by its similarity score of about 0.9 to 1.1 (see Example 5). This high similarity score indicates increased lysosomal trafficking and more rapid internalization of huR24-ADC when compared to huR24 not conjugated to a LD. This is particularly surprising given that much of ADC engineering is aimed at minimizing the differences in behavior between the naked and conjugated antibody. This surprising difference is advantageous in that it results in improved internalization of the huR24-ADC, as well as improved payload delivery by the huR24-ADC over what would be expected based on the behavior of huR24 alone.

In some aspects, the antibodies, or antigen-binding fragments thereof, described herein have a similarity score of approximately 0.6 with a lysosomal marker. In some aspects, the antibodies, or antigen-binding fragments thereof, described herein have a similarity score of approximately 0.7 with a lysosomal marker. In some aspects, the antibodies, or antigen-binding fragments thereof, described herein have a similarity score of approximately 0.8 with a lysosomal marker. In some aspects, the antibodies, or antigen-binding fragments thereof, described herein have a similarity score of approximately 0.6, 0.7, or 0.8 with a lysosomal marker at 100, 200, 300, or 350 minutes. In some aspects, the antibodies, or antigen-binding fragments thereof, described herein have a similarity score that is measured at 100, 200, 300, or 350 minutes. In some aspects, the antibodies, or antigen-binding fragments thereof, described herein have a similarity score of approximately 0.6 with a lysosomal marker at 100, 200, 300 or 350 minutes. In some aspects, the antibodies, or antigen-binding fragments thereof, described herein have a similarity score of approximately 0.7 with a lysosomal marker at 100, 200, 300 or 350 minutes. In some aspects, the antibodies, or antigen-binding fragments thereof, described herein have a similarity score of approximately 0.8 with a lysosomal marker at 100, 200, 300 or 350 minutes.

In some aspects, the antibodies, or antigen-binding fragments thereof, described herein have a similarity score that is measured at 100 minutes. In some aspects, the antibodies, or antigen-binding fragments thereof, described herein have a similarity score that is measured at 200 minutes. In some aspects, the antibodies, or antigen-binding fragments thereof, described herein have a similarity score that is measured at 300 minutes. In some aspects, the antibodies, or antigen-binding fragments thereof, described herein have a similarity score that is measured at 350 minutes.

In some aspects, the ADCs described herein have a similarity score of about 0.6 with a lysosomal marker. In some aspects, the ADCs described herein have a similarity score of about 0.7 with a lysosomal marker. In some aspects, the ADCs described herein have a similarity score of about 0.8 with a lysosomal marker. In some aspects, the ADCs described herein have a similarity score of about 0.9 with a lysosomal marker. In some aspects, the ADCs described herein have a similarity score of about 1.0 with a lysosomal marker. In some aspects, the ADCs described herein have a similarity score of about 1.1 with a lysosomal marker. In some aspects, the ADCs described herein have a similarity score that is measured at 100, 200, 300, or 350 minutes. In some aspects, the ADCs described herein have a similarity score of about 0.6, 0.7, 0.8, 0.9, 1.0 or 1.1 with a lysosomal marker at 100, 200, 300, or 350 minutes. In some aspects, the ADCs described herein have a similarity score of about 0.6 with a lysosomal marker at 100, 200, 300, or 350 minutes. In some aspects, the ADCs described herein have a similarity score of about 0.7 with a lysosomal marker at 100, 200, 300, or 350 minutes. In some aspects, the ADCs described herein have a similarity score of about 0.8 at 100, 200, 300, or 350 minutes. In some aspects, the ADCs described herein have a similarity score of about 0.9 with a lysosomal marker at 100, 200, 300, or 350 minutes. In some aspects, the ADCs described herein have a similarity score of about 1.0 with a lysosomal marker at 100, 200, 300, or 350 minutes. In some aspects, the ADCs described herein have a similarity score of about 1.1 with a lysosomal marker at 100, 200, 300, or 350 minutes. In other aspects, the ADCs described herein have a similarity score of about 0.9 to 1.1 with a lysosomal marker at 100, 200, or 300 minutes when compared to huR24.

In some aspects, the ADCs described herein have a similarity score that is measured at 100 minutes. In some aspects, the ADCs described herein have a similarity score that is measured at 200 minutes. In some aspects, the ADCs described herein have a similarity score that is measured at 300 minutes. In some aspects, the ADCs described herein have a similarity score that is measured at 350 minutes.

### Dosage

For the purpose of the present invention, the appropriate dosage of a GD3 antibody or a GD3 ADC will depend on the GD3 antibody or the GD3 ADC (or compositions thereof) employed, the type and severity of symptoms to be treated, whether the agent is administered for therapeutic purposes, previous therapy, the patient's clinical history and response to the agent, the patient's clearance rate for the administered agent, and the discretion of the attending physician. The clinician may administer a GD3 antibody or a GD3 ADC until a dosage is reached that achieves the desired result and beyond. Dose and/or frequency can vary over course of treatment, but may stay constant as well.

Empirical considerations, such as the half-life of the antibody or the Ab-ADC, generally will contribute to the determination of the dosage. For example, antibodies that are compatible with the human immune system, such as humanized antibodies or fully human antibodies, may be used to prolong half-life of the antibody and to prevent the antibody being attacked by the host's immune system.

In some aspects, the terminal plasma half-life in a mouse of the GD3-ADC described herein is one or more selected from the from the group consisting of about 1 day, about 1.5 days, about 2 days, about 2.5 days, about 3 days, about 3.5 days, about 4 days, about 4.5 days, about 5 days, about 5.5 days, and about 5.9 days. In some aspects, the terminal plasma half-life in a mouse of the GD3-ADC described herein is 5.6 days or 5.9 days. In some aspects the terminal plasma half-life in a rat of the GD3-ADC described herein is one or more selected from the from the group consisting of about 1 day, about 1.5 days, about 2 days, about 2.5 days, about 3 days, about 3.5 days, about 4 days, about 4.5 days, about 5 days, about 5.5 days, about 6 days, about 6.5 days, about 7 days, about 7.5 days, about 8 days, and about 8.5 days. In some aspects, the terminal plasma half-life in a rat of the GD3-ADC described herein is 8.1 days or 8.5 days. In some aspects, the terminal plasma half-life in a monkey of the GD3-ADC described herein is one or more selected from the group consisting of about 1 day, about 1.5 days, about 2 days, about 2.5 days, about 3 days, about 3.5 days, about 4 days, about 4.5 days, about 5 days, about 5.5 days, about 6 days, about 6.5 days, about 7 days, about 7.5 days, and about 7.7 days. In some aspects, the terminal plasma half-life in a monkey of the GD3-ADC described herein is 7 days, 7.6 days or 7.7 days. In some aspects, the terminal plasma half-life in a human of the ADCs described herein is one or more selected from the group consisting of about 1 day, about 1.5 days, about 2 days, about 2.5 days, about 3 days, about 3.5 days, about 4 days, about 4.5 days, about 5 days, about 5.5 days, about 6 days, about 6.5 days, and about 7 days. In some aspects, the terminal plasma half-life in a human of the ADCs described herein is 7 days.

In some aspects, the terminal plasma half-life in a mouse of the GD3 antibody, or antigen-binding fragments thereof, described herein is one or more selected from the from the group consisting of about 1 day, about 1.5 days, about 2 days, about 2.5 days, about 3 days, about 3.5 days, about 4 days, about 4.5 days, about 5 days, about 5.5 days, about 6 days, about 6.5 days, about 7 days, about 7.5 days, about 8 days, about 8.5 days, about 9 days, about 9.5 days, about 10 days, about 10.5 days, and about 10.9 days. In some aspects, the terminal plasma half-life in a mouse of the GD3 antibody, or antigen-binding fragments thereof, described herein is 10.6 days or 10.9 days. In some aspects, the terminal plasma half-life in a rat of the GD3 antibody, or antigen-binding fragments thereof, described herein is one or more selected from the from the group consisting of about 1 day, about 1.5 days, about 2 days, about 2.5 days, about 3 days, about 3.5 days, about 4 days, about 4.5 days, about 5 days, about 5.5 days, about 6 days, about 6.5 days, about 7 days, about 7.5 days, about 8 days, about 8.5 days, about 9 days, about 9.5 days, about 10 days, about 10.5 days, about 11 days, about 11.5 days, about 12 days, about 12.5 days, about 13 days, about 13.5 days, and about 13.7 days. In some aspects, the terminal plasma half-life in a rat of the GD3-antibody, or antigen-binding fragments thereof, described herein is 12.3 days or 13.7 days. In some aspects, the terminal plasma half-life in a monkey of the GD3-antibody, or antigen-binding fragments thereof, described herein is one or more selected from the from the group consisting of about 1 day, about 1.5 days, about 2 days, about 2.5 days, about 3 days, about 3.5 days, about 4 days, about 4.5 days, about 5 days, about 5.5 days, about 6 days, about 6.5 days, about 7 days, about 7.5 days, about 8 days, about 8.5 days, about 9 days, about 9.5 days, about 10 days, about 10.5 days, about 11 days, about 11.5 days, about 12 days, about 12.5 days, about 13 days, about 13.5 days, about 14 days, about 14.5 days, about 15 days, about 15.5 days, and about 16 days. In some aspects, the terminal plasma half-life in a monkey of the GD3-antibody, or antigen-binding fragments thereof, described herein is 10.8 days, 13 days or 16 days. In some aspects, the terminal plasma half-life in a human of the GD3-antibody, or antigen-binding fragments thereof, described herein is one or more selected from the from the group consisting of about 1 day, about 1.5 days, about 2 days, about 2.5 days, about 3 days, about 3.5 days, about 4 days, about 4.5 days, about 5 days, about 5.5 days, about 6 days, about 6.5 days, about 7 days, about 7.5 days, about 8 days, about 8.5 days, about 9 days, about 9.5 days, about 10 days, about 10.5 days, about 11 days, about 11.5 days, about 12 days, about 12.5 days, about 13 days, about 13.5 days, about 14 days, about 14.5 days, about 15 days, about 15.5 days, and about 16 days. In some aspects, the terminal plasma half-life in a human of the GD3-antibody, or antigen-binding fragments thereof, described herein is 10.8 days, 13 days or 16 days.

Frequency of administration may be determined and adjusted over the course of therapy, and is generally, but not necessarily, based on treatment and/or suppression and/or amelioration and/or delay of symptoms, e.g., tumor growth inhibition or delay, etc. Alternatively, sustained continuous release formulations of GD3 antibody or GD3-ADC may be appropriate. Various formulations and devices for achieving sustained release are known in the art.

GD3 antibodies or the GD3-ADC of the invention can be administered using any suitable method, including by injection (e.g., intraperitoneally, intravenously, subcutaneously, intramuscularly, etc.). The GD3 antibody or the GD3-ADC can also be administered via inhalation, as described herein. Generally, for administration of a GD3 antibody or a GD3 ADC, a dosage can be about 0.5 mg/kg, about 1 mg/kg, about 2.5 mg/kg, about 5 mg/kg, about 10 mg/kg, and about 25 mg/kg. A typical daily dosage might range from about any of 3 μg/kg, to 30 μg/kg, to 300 μg/kg, to 3 mg/kg, to 30 mg/kg, to 100 mg/kg or more, depending on the factors mentioned above. For repeated administrations over several days or longer, depending on the condition, the treatment is sustained until a desired suppression of symptoms occurs or until sufficient therapeutic levels are achieved, for example, to inhibit or delay tumor growth/progression or metastasis of cancer cells. An exemplary dosing regimen includes administering an initial dose of about 2 mg/kg, followed by a weekly maintenance dose of about 1 mg/kg of the GD3 antibody or GD3 ADC, or followed by a maintenance dose of about 1 mg/kg every other week. Other exemplary dosing regimens include administering increasing doses (e.g., initial dose of 1 mg/kg and gradual increase to one or more higher doses every week or longer time period). Other dosage regimens may also be useful, depending on the pattern of pharmacokinetic decay that the practitioner wishes to achieve. For example, in some aspects of the invention, dosing from one to four times a week is contemplated. In other aspects, dosing once a month or once every other month or every three months is contemplated, as well as weekly, bi-weekly and every three weeks. The progress of this therapy may be easily monitored by conventional techniques and assays. The dosing regimen (including the GD3 antibody or the GD3-ADC used) can vary over time.

In some aspects of the invention, dosages for a GD3 antibody or a GD3-ADC may be determined empirically in individuals who have been given one or more administration(s) of the GD3 antibody or the GD3-ADC. Individuals are given incremental dosages of a GD3 antibody or a GD3 ADC. To assess efficacy, an indicator of the disease can be followed.

For in vitro and in vivo applications, GD3 antibody or GD3-ADC are provided or administered in an effective dosage. In a clinical context, an effective dosage of drug, compound, or pharmaceutical composition is an amount sufficient to accomplish prophylactic or therapeutic treatment either directly or indirectly. An effective dosage can be administered in one or more administrations. An effective dosage of a drug, compound, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved. For detection of GD3-positive cells using the disclosed GD3 antibodies or ADCs, a detectable amount of a composition of the invention is administered to a subject, i.e., a dose of the conjugate such that the presence of the conjugate may be determined in vitro or in vivo.

For example, when administered to a cancer patient, an effective amount includes an amount sufficient to elicit anti-cancer activity, including cancer cell cytolysis, inhibition of cancer cell proliferation, induction of cancer cell apoptosis, reduction of cancer cell antigens, delayed tumor growth, and/or inhibition of metastasis. Decreased tumor size is well accepted as a clinical surrogate marker for efficacy. Another well accepted marker for efficacy is progression-free survival. A GD3 antibody or GD3-ADC of the invention generally demonstrate at least a 25% improvement in key efficacy parameters, such as improvement in median survival, time to tumor progression, and overall response rate.

The GD3 antibody or the GD3-ADC can be administered to an individual via any suitable route. It should be understood by persons skilled in the art that the examples described herein are not intended to be limiting but to be illustrative of the techniques available. Accordingly, in some aspects of the invention, the GD3 antibody or the GD3 antibody conjugate is administered to an individual in accord with known methods, such as intravenous administration, e.g., as a bolus or by continuous infusion over a period of time, by intramuscular, intraperitoneal, intracerebrospinal, intracranial, transdermal, subcutaneous, intra-articular, sublingually, intrasynovial, via insufflation, intrathecal, oral, inhalation or topical routes. Administration can be systemic, e.g., intravenous administration, or localized. Commercially available nebulizers for liquid formulations, including jet nebulizers and ultrasonic nebulizers are useful for administration. Liquid formulations can be directly nebulized and lyophilized powder can be nebulized after reconstitution. Alternatively, the GD3 antibody or the GD3 ADC can be aerosolized using a fluorocarbon formulation and a metered dose inhaler, or inhaled as a lyophilized and milled powder.

In some aspects of the invention, the GD3 antibody or the GD3 ADC is administered via site-specific or targeted local delivery techniques. Examples of site-specific or targeted local delivery techniques include various implantable depot sources of the GD3 antibody or the GD3 ADC or local delivery catheters, such as infusion catheters, indwelling catheters, or needle catheters, synthetic grafts, adventitial wraps, shunts and stents or other implantable devices, site specific carriers, direct injection, or direct application. See, e.g. PCT International Publication No. WO 2000/53211 and U.S. Pat. No. 5,981,568.

Administration of a GD3 antibody or a GD3 ADC in accordance with the method in the present invention can be continuous or intermittent, depending, for example, upon the recipient's physiological condition, whether the purpose of the administration is therapeutic or prophylactic, and other factors known to skilled practitioners. The administration of a GD3 antibody or a GD3 ADC may be essentially continuous over a preselected period of time or may be in a series of spaced doses.

### Kits

The invention also provides kits or an article of manufacture comprising an antibody, or antigen binding fragment thereof, of the invention, and instructions for use. Accordingly, in some embodiments, provided is a kit or an article of manufacture, comprising a container, a composition within the container comprising a GD3 antibody or a GD3 ADC, and a package insert containing instructions to administer a therapeutically effective amount of the anti-IL-33 antagonist antibody for treatment of a patient in need thereof.

In certain embodiments, the kit can contain both a first container having a dried protein and a second container having an aqueous formulation. In certain embodiments, kits containing single and multi-chambered pre-filled syringes (e.g., liquid syringes and lyosyringes) are included.

The instructions relating to the use of antibodies or antigen binding fragments thereof of the invention generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). The container may further comprise a second pharmaceutically active agent.

Kits may optionally provide additional components such as buffers and interpretive information. Normally, the kit comprises a container and a label or package insert(s) on or associated with the container.

### Definitions

“About” or “approximately,” unless otherwise defined herein, when used in connection with a measurable numerical variable, refers to the indicated value of the variable and to all values of the variable that are within the experimental error of the indicated value (e.g. within the 95% confidence interval for the mean) or within 10 percent of the indicated value, whichever is greater. Numeric ranges are inclusive of the numbers defining the range.

As used herein, “vector” means a construct, which is capable of delivering, and, preferably, expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

The term “identity,” as known in the art, refers to a relationship between the sequences of two or more polypeptide molecules or two or more nucleic acid molecules, as determined by comparing the sequences. In the art, “identity” also means the degree of sequence relatedness between polypeptide or nucleic acid molecule sequences, as the case may be, as determined by the match between strings of nucleotide or amino acid sequences. “Identity” measures the percent of identical matches between two or more sequences with gap alignments addressed by a particular mathematical model of computer programs (i. e. “algorithms”).

The term “similarity” is a related concept, but in contrast to “identity”, refers to a measure of similarity which includes both identical matches and conservative substitution matches. Since conservative substitutions apply to polypeptides and not nucleic acid molecules, similarity only deals with polypeptide sequence comparisons. If two polypeptide sequences have, for example, 10 out of 20 identical amino acids, and the remainder are all nonconservative substitutions, then the percent identity and similarity would both be 50%. If in the same example, there are 5 more positions where there are conservative substitutions, then the percent identity remains 50%, but the percent similarity would be 75% (15 out of 20). Therefore, in cases where there are conservative substitutions, the degree of similarity between two polypeptide sequences will be higher than the percent identity between those two sequences.

The use of the term “or” in the claims is used to mean “and/or” unless explicitly indicated to refer to alternatives only or the alternatives are mutually exclusive, although the disclosure supports a definition that refers to only alternatives and “and/or.” As used herein the specification, “a” or “an” may mean one or more, unless clearly indicated otherwise. As used herein in the claim(s), when used in conjunction with the word “comprising”, the words “a” or “an” may mean one or more than one. As used herein “another” may mean at least a second or more. Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. The words “comprises/comprising” and the words “having/including” when used herein with reference to the present invention are used to specify the presence of stated features, integers, steps or components but does not preclude the presence or addition of one or more other features, integers, steps, components or groups thereof.

### Biological Deposits

Representative materials of the present invention were deposited in the American Type Culture Collection, 10801 University Boulevard, Manassas, Va. 20110-2209, USA, on Apr. 11, 2017. Vector huR24-VH having ATCC Accession No. PTA-124057 comprises a DNA insert encoding the heavy chain variable region of antibody huR24, and vector huR24-VL having ATCC Accession No. PTA-124058 comprises a DNA insert encoding the light chain variable region of antibody huR24. The deposits were made under the provisions of the Budapest Treaty on the International Recognition of the Deposit of Microorganisms for the Purpose of Patent Procedure and Regulations thereunder (Budapest Treaty). This assures maintenance of a viable culture of the deposit for 30 years from the date of deposit. The deposit will be made available by ATCC under the terms of the Budapest Treaty, and subject to an agreement between Pfizer Inc. and ATCC, which assures permanent and unrestricted availability of the progeny of the culture of the deposit to the public upon issuance of the pertinent U.S. patent or upon laying open to the public of any U.S. or foreign patent application, whichever comes first, and assures availability of the progeny to one determined by the U.S. Commissioner of Patents and Trademarks to be entitled thereto according to 35 U.S.C. Section 122 and the Commissioner's rules pursuant thereto (including 37 C.F.R. Section 1.14 with particular reference to 886 OG 638).

The assignee of the present application has agreed that if a culture of the materials on deposit should die or be lost or destroyed when cultivated under suitable conditions, the materials will be promptly replaced on notification with another of the same. Availability of the deposited material is not to be construed as a license to practice the invention in contravention of the rights granted under the authority of any government in accordance with its patent laws

### EXAMPLES

The following examples are offered for illustrative purposes only, and are not intended to limit the scope of the present invention in any way. Indeed, various modifications of the invention in addition to those shown and described herein will become apparent to those skilled in the art from the foregoing description and fall within the scope of the appended claims.

### Example 1 Humanization of Murine Anti-GD3 Antibodies

Murine R24 (mR24) is a murine antibody that binds GD3 (SEQ ID NOs: 17 and 19). To minimize the risk of immunogenicity, an attempt was made to humanize mR24 for further therapeutic development. An earlier attempt to humanize mR24 is disclosed in WO2008/101234. The CDRs of murine antibody mR24 were identified using Kabat. Humanization of mR24 was first attempted using the semi-empirical framework shuffle approach. In this approach, human germline frameworks with a high degree of homology with mR24 sequences, VL frameworks V1-12 and V3-11 and VH framework V3-7, were selected. The selected frameworks were then used as the acceptor framework for mR24 without any backmutations in the framework region. The CDRs of mR24 were then inserted into the acceptor frameworks, and antibody proteins were generated and assessed for their expression yield, purification properties, and GD3 target binding activity. After screening, two advanced humanization variants with high GD3 binding activity, overall expression yields, and stability, hAb 21 (SEQ ID NOs: 30 and 31, VL framework V3-11 and VH framework V3-7) and hAb3 (SEQ ID NOs: 32 and 33, VL framework V1-12 and VH framework V3-7), were further evaluated for their aggregation level after purification. As shown in Table 3, the affinity of hAb21 and hAb3 were 3-to-6 fold lower than chR24 (SEQ ID NOs: 28 and 29). In addition, as shown in Table 4, the expression yields were no more than half of the expression yields of chR24.

TABLE 2
Nomenclature of humanized versions based on framework shuffle

| | |
|---|---|
| KM641 | murine IgG3, kappa, or mR24 9 (SEQ ID NOs: 17 and 19) |
| KM871 | chR24 (hAb.51) chimeric version (hIgG1, kappa) of KM641 |
| | (SEQ ID NOs: 28 and 29) |
| hAb 21 | fully humanized mR24 based on |
| | framework shuffle (SEQ ID NOs: 30 and |
| | 31, VL framework V3-11 and VH framework V3-7) |
| hAb 3 | fully humanized mR24 based on |
| | framework shuffle (SEQ ID NOs: 32 and 33, |
| | VL framework V1-12 and VH framework V3-7) |

TABLE 3
Estimation of binding affinity by Octet

| | | SD KD | | SD Kon | Kdis | SD kdis |
|---|---|---|---|---|---|---|
| Average± | KD (M) | (M) | Kon(1/Ms) | (1/Ms) | (1/s) | (1/s) |
| KM871 | 9.02E−09 | 6.77E−09 | 1.33E+06 | 1.45E+06 | 5.96E−03 | 2.16E−03 |
| Ab 3 | 5.97E−08 | 3.51E−08 | 7.54E+04 | 6.81E+04 | 2.78E−03 | 6.24E−04 |
| (concentrated at | | | | | | |
| 21.2 mg/ml, in | | | | | | |
| Tier-1 buffer) | | | | | | |
| hAb 3 dialyzed | 5.58E−08 | 3.08E−08 | 3.77E+04 | 2.92E+04 | 1.44E−03 | 2.84E−04 |
| in PBS | | | | | | |
| hAb 21 | 3.11E−08 | 1.44E−08 | 4.46E+04 | 2.69E+04 | 1.11E−03 | 2.03E−04 |
| (concentrated at | | | | | | |
| 27.8 mg/ml, in | | | | | | |
| Tier-1 buffer) | | | | | | |
| hAb 21 dialyzed | 2.89E−08 | 1.55E−08 | 5.97E+04 | 3.27E+04 | 1.36E−03 | 2.50E−04 |
| in PBS | | | | | | |

TABLE 4
Expression yields of advanced humanized leads.

| | Expression yield | |
|---|---|---|
| Antibody | (mg/L culture) | HMA Analytical SEC |
| hAb 3 | 8.61 | <5% |
| hAb 21 | 6.87 | <5% |
| KM871 (chR24) | 15.6 | <5% |

### Example 2 Selection of Frameworks for Humanization of Murine Anti-GD3 Antibodies

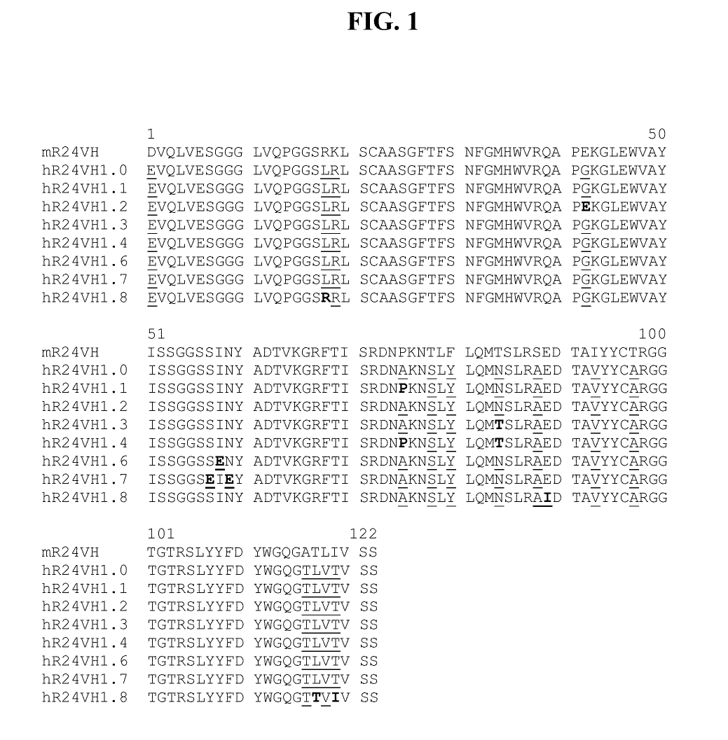

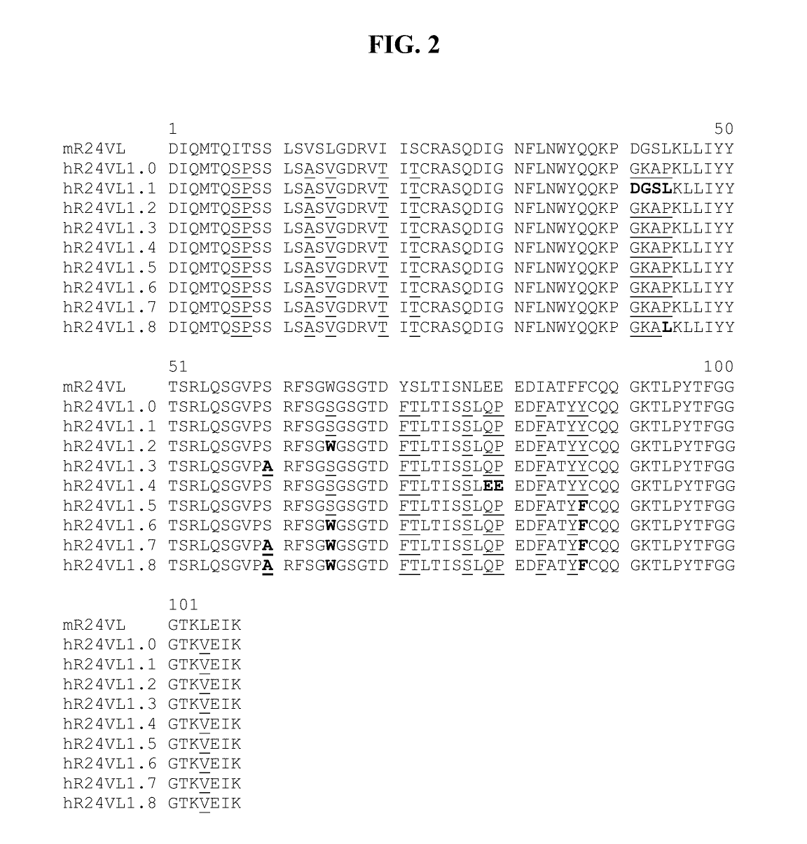

In an effort to maximize the expression, manufacturability and biophysical properties of the humanized anti-GD3 antibody, the CDR regions of mR24 were grafted onto a set of alternative human frameworks, VH framework VH3-DP54_JH4 and VL framework VK3-DPK9_JK4. This CDR grafted version is designated as hR24VH1.0/VL1.0. The sequences of hR24VH1.0 (SEQ ID NO: 34) and hR24VL1.0 (SEQ ID NO: 36) are shown in FIGS. 1 and 2. However, the affinity, expression, manufacturability and certain biophysical properties of hR24VH1.0/VL1.0 were lower than chR24.

A more detailed investigation was undertaken to explore whether a solution could be found to the problem of lower parameters compared with the mouse parental antibody mR24 as a mouse-human chimera (chR24), based on structural modeling analysis using X-ray structures of mR24, two crystal structures of the mouse (PDB id: 1 R24) and chimeric (PDB id: 1 BZ7) mR24 antibodies were available (Kaminski et al., 1999, J Biol Chem 274(9):5597-5604). It was observed that homotypic interface, where an antiparallel beta-sheet is formed between CDR-H2 regions (CDR-H2 residues H-56 to H-58, by Kabat numbering) of a pair of VH domains, is conserved in both of these structures. This interface was identified structurally by Kaminski et al (Kaminski et al., 1999, J Biol Chem 274(9):5597-5604) and previously identified in binding studies by Yan et al (Yan et al., 1996, J Immunol 157(4):1582-1588). Both show that this homotypic interface plays a role GD3 binding. Heavy chain variants hR24VH1.6 (I58E)) and hR24VH1.7 (S57E/N59E), as shown in FIG. 1, removed the homotypic interface via charge-charge repulsion. Both variants showed significant loss of activity as described in table 5 below. These data demonstrate that conservation of the homotypic interface is required for humanized antibodies with the desired activity.

There were three sites in VH framework VH3-DP54_JH4, H19, H74, and H82a (by Kabat numbering), that faced the homotypic interface and were different from the corresponding sites in the chR24 framework. Although these residues did not interact at the homotypic interface, H74 had the potential to alter the loop that interacted with CDR-H1 and CDR-H2, as described further in Example 3 below.

### Example 3 Selection of Mutations for Humanization of Murine Anti-GD3 Antibodies

To explore the expression, manufacturability and biophysical properties further, a homology model of hR24VH1.0/VL1.0 was generated to compare the crystal structure of hR24VH1/VL1.0 to the crystal structure of chR24. All sites that differed were categorized based upon surface exposure, potential structural changes upon mutation (particularly in the CDR regions), potential interactions with the antigen or with the homotypic interface and rarity of a residue type at a site. Of the many potential mutations, see Table 5, two were experimentally demonstrated to significantly improve the activity to the levels of the chimera. Using Kabat numbering, these were the serine to tryptophan mutation at position 65 of the variable region of the light chain (S_L65_W) and the alanine to proline mutation at position 74 of the variable region of the heavy chain (A_H74_P). Both of these positions are on the surface and increased the surface hydrophobicity. Neither of these sites are canonical mutations that are common in humanization of mouse antibodies (Lo, 2004, Methods in Molecular Biology 248:135-159; O'Brien and Jones, 2003, Methods in Molecular Biology 207:81-100).

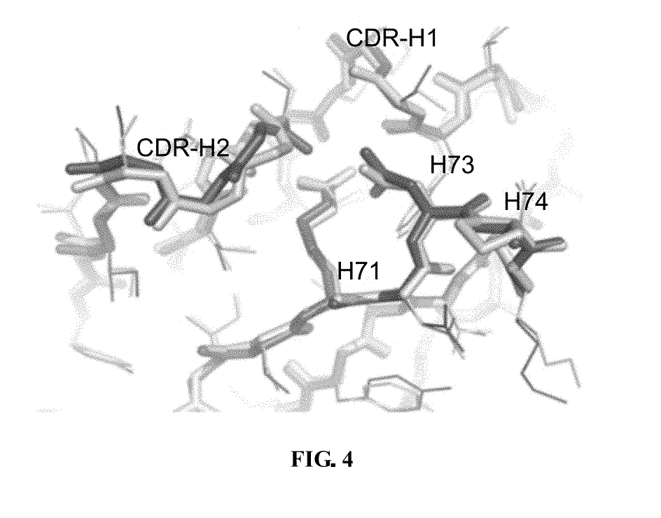

The identification and selection of proline at position 74 was also unpredictable and surprising, especially given the propensity for proline side chains to effect deleterious structural and conformational alterations in relation to the relatively small side chain of alanine. Heavy chain mutation, i.e., alanine to proline at amino acid residue 74 of the heavy chain (A_H74_P), was identified by its proximity to the homotypic interface and its proximity to two structurally important sites (heavy chain sites H71 and H73) which have been described as residues important for the proper conformational structure of the CDRH1 and CDRH2 loops (Foote and Winter, 1992). It was postulated that mutations at the heavy chain H74 position could alter the local structure of that portion of framework 3 and alter the conformations of the heavy chain H71 and/or H73 sites (FIG. 4). However, it was unexpected and surprising that substitution of alanine, which allows the greatest conformational freedom, to a proline, which introduces a classic “kink” in the conformation of proteins, would result in the improved binding characteristics observed in huR24. Nonetheless, humanized mR24 variant hR24 VH1.1 (SEQ ID NO: 1) containing this A_H74_P mutation (FIG. 1) was unexpectedly shown to have increased affinity and activity compared to the parent antibody comprising alanine at this position.

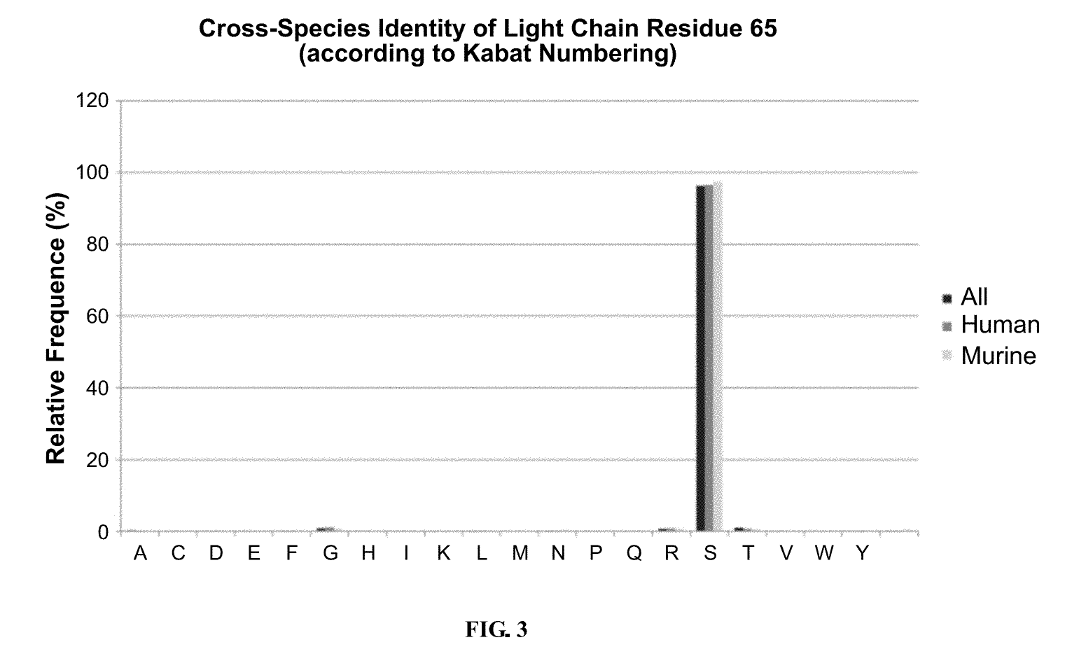

The identification and selection of tryptophan at position 65 was particularly surprising. The distribution of the S_L65_W mutation is shown in FIG. 3 using sequences in the Abysis database v2.3.3 (Abhinandan and Martin, 2008). It is clear from this data that this site is quite conserved as serine. For all antibodies serine occurs >96% of the time, and >97% of the time in human and murine antibodies. Other than in the mR24 antibody, tryptophan only occurs in 4 other antibodies out of approximately 25,000 total sequences. Humanized R24 variant hR24 VL1.2 (SEQ ID NO: 9) also contained this S_L65_W mutation (see FIG. 2) and this mutation was experimentally shown to increase the affinity and activity compared with the parental antibody comprising serine at L65.

The cDNAs of the humanized mR24 variants described in Table 5 were synthesized and then fused in frame with the human IgG1 constant regions, for the heavy chain, and human kappa, for the light chain, within mammalian expression vectors. These variants, including hR24vh1.1/vl1.2 (huR24, SEQ ID NOs: 1 and 9), were assessed for binding, CDC, ADCC, ADC feasibility, PACS, binding activity with plate bound GD3, and binding activity with cell surface expressed GD3.

As shown in Table 5 below, huR24vh1.1 showed an increased combination of binding, ADC feasibility, PACS, and GD3 ELISA activity when compared to the other mR24 VH variants. hR24vl1.2 also showed an increased combination of binding, CDC, ADCC, ADC feasibility, and PACS activity when compared to the other mR24 VL variants.

TABLE 5
Humanized Variants of mR24 using Frameworks VH framework VH3-DP54_JH4 and VL framework VK3-DPK9_JK4

Humanized

| mR24 | | | | | | | Cell Based ELISA | | |
|---|---|---|---|---|---|---|---|---|---|
| Variant | Mutation(s) | Binding | CDC | ADCC | ADC | PACS | SK-MEL-28 | G3-61 | GD3 ELISA |

VL variants paired with VH 1.0

| | | | | | | | | | |
|---|---|---|---|---|---|---|---|---|---|
| hR24VL1.0 | none | + | − | + | + | + | ++ | + | +++ |
| hR24VL1.1 | G41D, K42G, A43S, P44L | ++ | − | + | − | + | ++ | ++ | +++ |
| hR24VL1.2 | S65W | ++ | ++ | ++ | +++ | ++ | ++ | ++ | ++ |
| hR24VL1.3 | S60A | + | − | + | + | + | ++ | + | +++ |
| hR24VL1.4 | Q79E_P80E | − | − | − | + | + | ++ | + | ++ |
| hR24VL1.5 | Y87F | + | − | + | ++ | + | ++ | ++ | ++ |
| hR24VL1.6 | S65W_Y87F | + | + | + | + | + | ++ | ++ | ++ |
| hR24VL1.7 | S65W_S60A_Y87F | + | + | + | + | + | ++ | ++ | ++ |
| hR24VL1.8 | P44L_S65W_S60A_Y87F | − | − | − | − | +/− | + | + | + |

VH variants paired with VL 1.0

| | | | | | | | | | |
|---|---|---|---|---|---|---|---|---|---|
| hR24VH1.1 | A74P | +++ | + | + | +++ | ++ | +++ | ++ | ++++ |
| hR24VH1.2 | G42E | ++ | + | + | − | + | +++ | ++ | +++ |
| hR24VH1.3 | N84T | ++ | + | + | + | + | +++ | + | +++ |
| hR24VH1.4 | N84T_A74P | + | + | + | +++ | + | +++ | ++ | +++ |
| hR24VH1.6 | I58E | − | − | − | − | + | + | + | + |
| hR24VH1.7 | S57E_N59E | − | − | − | − | − | − | − | − |
| hR24VH1.8 | L18R_E89I_L117T_T119I | + | − | − | + | + | + | + | + |

For small scale antibody production, each huR24 variant heavy chain, in pSMED2, and each corresponding huR24 variant light chain, in pSMEN3, were co-transfected into HEK-293F cells (Invitrogen, Carlsbad, Calif.) according to the manufacturer's protocol and conditioned medium (CM) was collected 5-7 days later for antibody purification. For large scale antibody production, heavy and light chain expression constructs were co-transfected into Chinese Hamster Ovary cells (CHO), and selectable markers for both constructs, dihydrofolate reductase for pSMED2, and neomycin resistance for pSMEN3, were used to select for cells with the expression constructs stably incorporated into their genome. The cells stably harboring the antibody genes of interest were then expanded and large scale conditioned medium was collected. Harvested CM was clarified using a 5 μm and 0.2 μm depth filtration, followed by 5-fold concentration via tangential flow filtration. HuR24 demonstrated good expression yields following transient transfection in HEK293 cells (around 40 mg/L) or stable transfection in CHO cells (around 600 mg/L).

There is a positive correlation between the thermal stability of a protein or protein domain with the overall stability of the protein or protein domain. A higher melting temperature of a protein or protein domain often provides improved manufacturability and longer shelf life. Differential scanning calorimetry (DSC) was used to assess the thermal stability of huR24. Antibody samples were diluted in designated buffers as listed below to 0.3 mg/mL in a volume of 250 μL. The corresponding formulation buffer blank was used for the reference sample. Both samples were thoroughly degassed using a MicroCal ThermoVac Sample Degassing and Thermostat (Microcal, Inc, Northampton, Mass.) set to 8° C. Samples were dispensed into the appropriate cells of a MicroCal VP-DSC Capillary Cell MicroCalorimter (MicroCal, Inc, Northampton, Mass.). Samples were equilibrated for 4 minutes at 15° C. and then scanned up to 100° C. at a rate of 100° C. per hour. A filtering period of 20 seconds was selected. Raw data was baseline corrected and the protein concentration was normalized. Origin Software (OriginLab Corporation, Northampton, Mass.) was used to fit the data to an MN2-State Model with an appropriate number of transitions. As shown in Table 6, huR24 had good thermal stability in all buffers tested, with the melting temperature of the Fab region above 80° C. These results demonstrate that desirable characteristics of murine R24 have been preserved during humanization and that huR24 is a potential therapeutic for GD-3 expressing tumors.

TABLE 6
Thermal stability (DSC) analysis of
humanized anti-GD3 antibody huR24

| | Tm (° C.) ± Standard Deviation | | |
|---|---|---|---|
| Buffer | Tm1 | Tm2 | Tm3 |
| Tris | 73.6 ± 0.1 | 87.2 ± 0.3 | 91.9 ± 0.1 |
| His | 71.0 ± 0.1 | 86.7 ± 0.3 | 91.7 ± 0.1 |
| Succinate | 72.5 ± 0.1 | 87.0 ± 0.2 | 92.0 ± 0.1 |
| Citrate | 70.4 ± 0.1 | 86.4 ± 0.2 | 91.6 ± 0.1 |
| Acetate | 65.5 ± 0.2 | 84.2 ± 0.3 | 90.4 ± 0.1 |

DSC = Differential scanning calorimetry; Tm = Melting temperature of a specific domain in question. The melting temperatures of distinct domains of antibody huR24VH1.1/VL1.2 were shown. Tm1 represents the melting temperature for heavy chain constant region domain 2 (CH2) and Tm3 for CH3. Tm2 represents the melting temperature of Fab (the entity of an antibody composed of the variable regions of both heavy and light chains together with the constant region of light chain and the heavy chain constant region 1).

### Example 4 Binding Properties of Anti-GD3 Antibodies

Cell Binding Activity by ELISA

Endogenous GD3 expressing cells (SK-MEL melanoma cell line or G361) were plated at 50,000 cells/well in 100 μL appropriate medium (described below) in 96 well cell culture plates (BD Biosciences) one day prior. On the day of the ELISA, culture medium was removed from the wells, and the cells were fixed by adding 50 μL/well of fixation solution (Cytofix, BD Biosciences, Cat No 554655) and incubated for 30 minutes on ice. Following incubation, the plates were washed twice with PBS and were left in PBS supplemented with 1% of bovine serum albumin (BSA). Humanized variants of mR24 were then serially diluted 1:3 in PBS plus calcium/magnesium (PBS-Ca²⁺/Mg²⁺), 1% BSA were applied to the plates. The plates were then incubated on ice for 1 hour and then washed 4 times with PBS-Ca²⁺/Mg²⁺. Horseradish Peroxidase (HRP)-conjugated secondary antibody (goat anti-human IgG Fragment Crystalizable region (Fc), Jackson ImmunoResearch, Cat No 109-035-098) diluted 1:5000 in PBS-Ca²⁺/Mg²⁺ with 1% BSA was then applied for 1-hour incubation on ice. Plates were washed again as described above and TMB substrate solution (3,3′,5,5′-tetramethylbenzidine (TMB); BioFX Labs, Owing Mills, Md.) was added for 10 minutes, followed by 0.18 M H₂SO₄. Absorbance at OD450 nM was then measured and data were plotted and analyzed with Microsoft Excel and Graphpad-Prism software.

The cell growth medium for SK-MEL028 was DMEM supplemented with 10% FBS, non-essential amino acids and penicillin-streptomycin-Glutamate (Invitrogen, Carlsbad, Calif.). The cell growth medium for G361 was McCoy 5a supplemented with 10% FBS, non-essential amino acids and penicillin-streptomycin-Glutamate (Invitrogen, Carlsbad, Calif.).

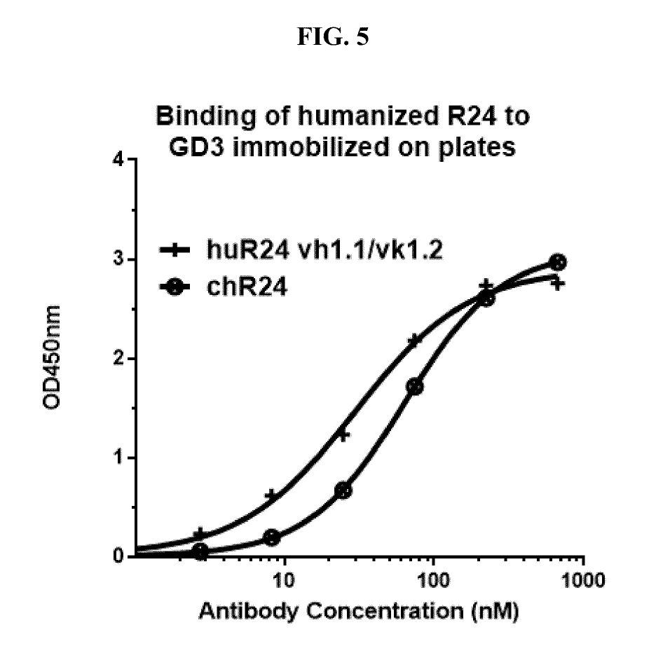

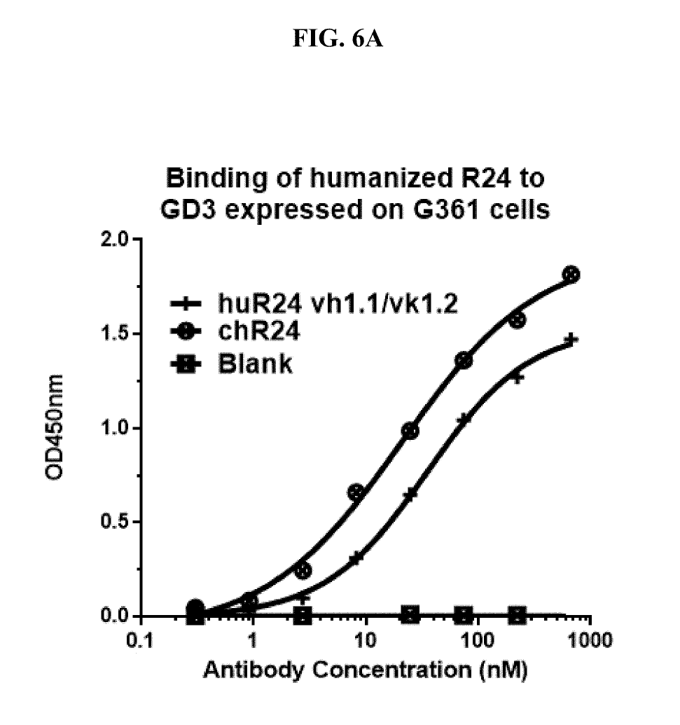

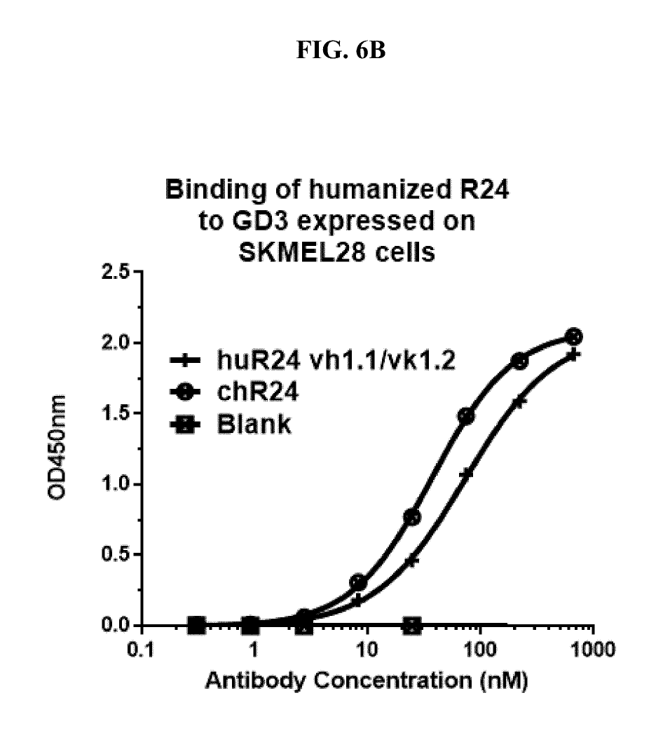

Purified recombinant antibody was used to evaluate the GD3 binding properties of huR24 in both plate based and cell based ELISAs. As shown in (FIG. 5), huR24 exhibited better binding capacity than chimeric antibody chR24 (SEQ ID NOs: 29 and 30). HuR24 showed comparable binding capacity to chR24 in endogenous cell surface-expressed GD3 assays on both G361 and SK-MEL028 tumor cell lines (FIGS. 6A and 6B, respectively). These data suggest that huR24 has exceeded the binding activity of its parental mouse antibody mR24 (made as a human-mouse chimera chR24). Thus, the mutations have surprisingly resulted in a humanized antibody with better binding characteristics than its parental mouse antibody.

Cell Binding Activity of hu24 by Flow Cytometry

HuR24 was examined for cell surface binding to live cells by flow cytometry. To confirm specificity, binding of huR24 was assessed on target-negative human colorectal adenocarcinoma cells (COLO-205) and on a panel of metastatic melanoma cell lines with varying levels of GD3 expression (Table 7). huR24 showed no binding to COLO-205 cells. In contrast, hu24 bound to metastatic melanoma cell lines with variable relative fluorescence intensities.

TABLE 7
Flow Cytometry Cell Surface Binding of hu24
on Human Metastatic Melanoma Cell Lines.

| | MFIR relative to | | MFIR relative to |
|---|---|---|---|
| Cell Line | isotype control | Cell Line | isotype control |
| G361 | 28 ^{a} | SK-MEL-28 ^{b} | 39 |
| M19-MEL | 25 ^{b} | SK-MEL-30 ^{a} | 15 |
| Malme3M | 11 ^{a} | SK-MEL-5 ^{b} | 73 |
| MeWo | 15 ^{a} | UACC-257 ^{b} | 13 |
| NCI-M14 | 14 ^{b} | UACC-62 ^{b} | 7 |
| SK-MEL-19 | 21 ^{a} | UCSD-242L ^{b} | 8 |
| SK-MEL-2 | 4 ^{b} | UCSD354L ^{b} | 5 |

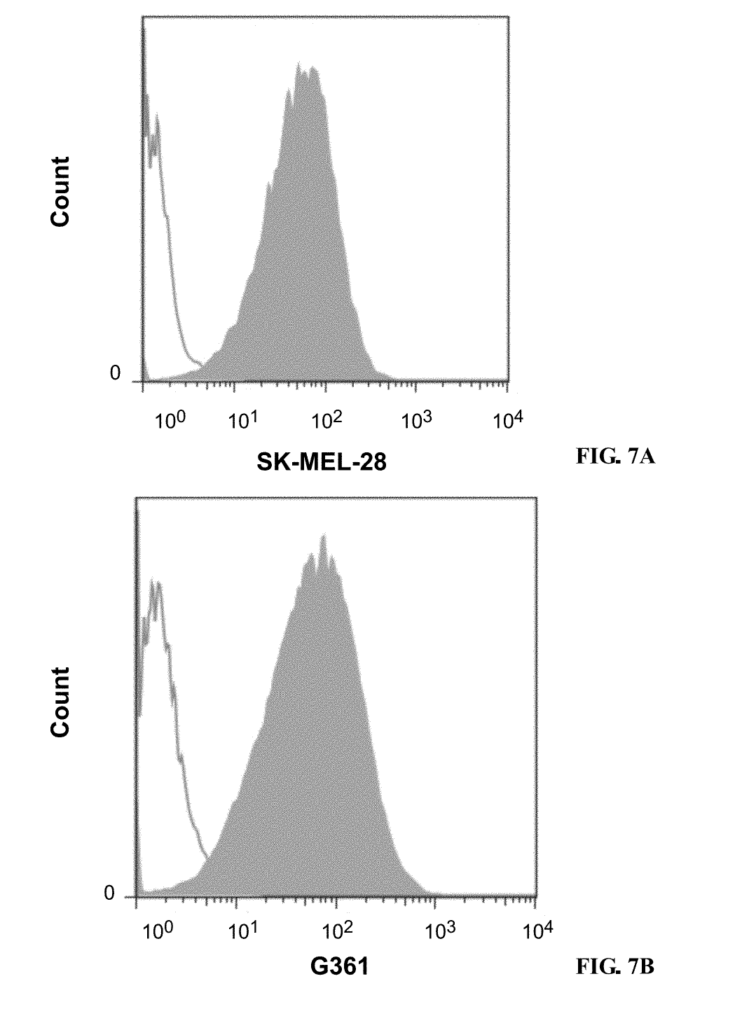

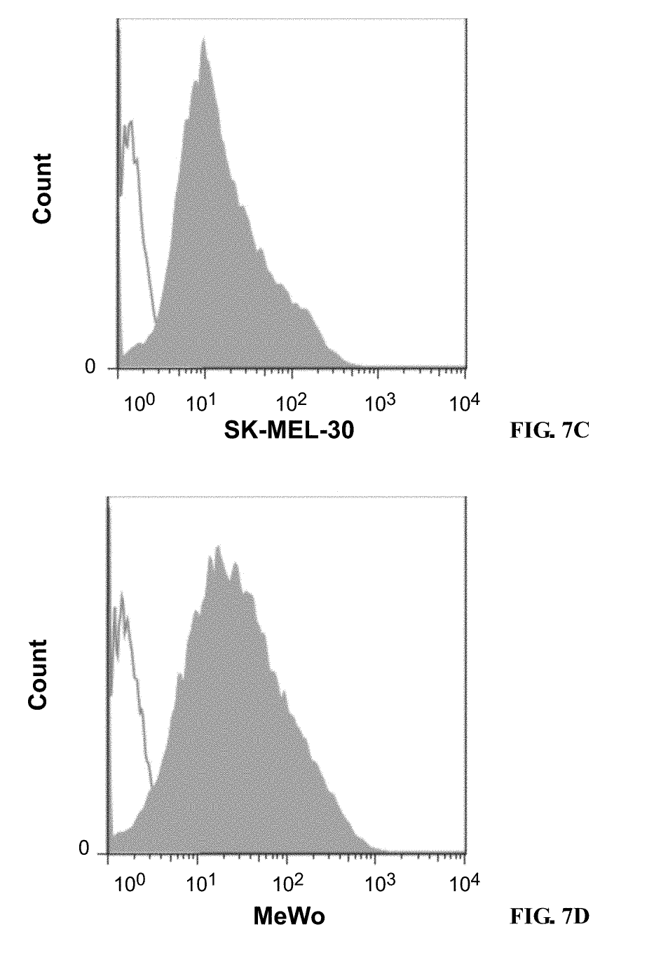

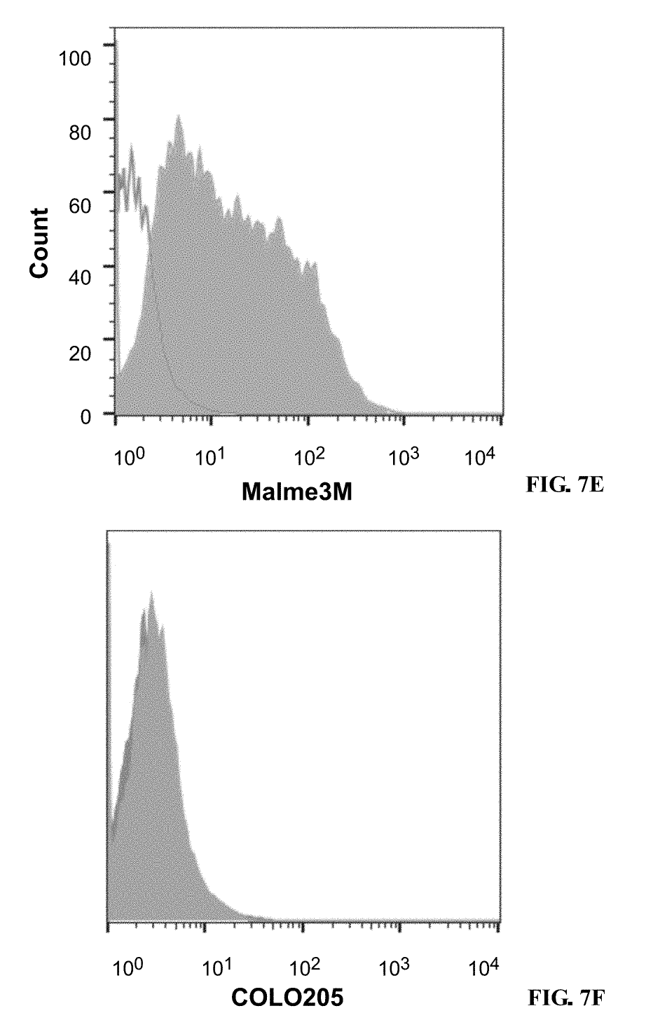

The Median Fluorescence Intensity Ratio (MFIR=Ratio of MFI for unconjugated huR24 to MFI of one of the isotype controls, control monoclonal antibody conjugated to linker payload (b) or unconjugated control monoclonal antibody (a) was assessed on a panel of GD3-positive tumor cell lines (Table 7 and FIGS. 7A-7E) wherein MFI is calculated as the area under the curve of each graph shown. The isotype control antibody did not exhibit appreciable binding to any of the cell lines. These results demonstrate that cell-surface GD3 expressed on melanoma lines is specifically bound by huR24, and further suggest that huR24 is a potential therapeutic for treating GD3-expressing tumors at least since it binds to the surface of GD3 surface expressing cells.

### Example 5 Internalization of Anti-GD3 Antibodies

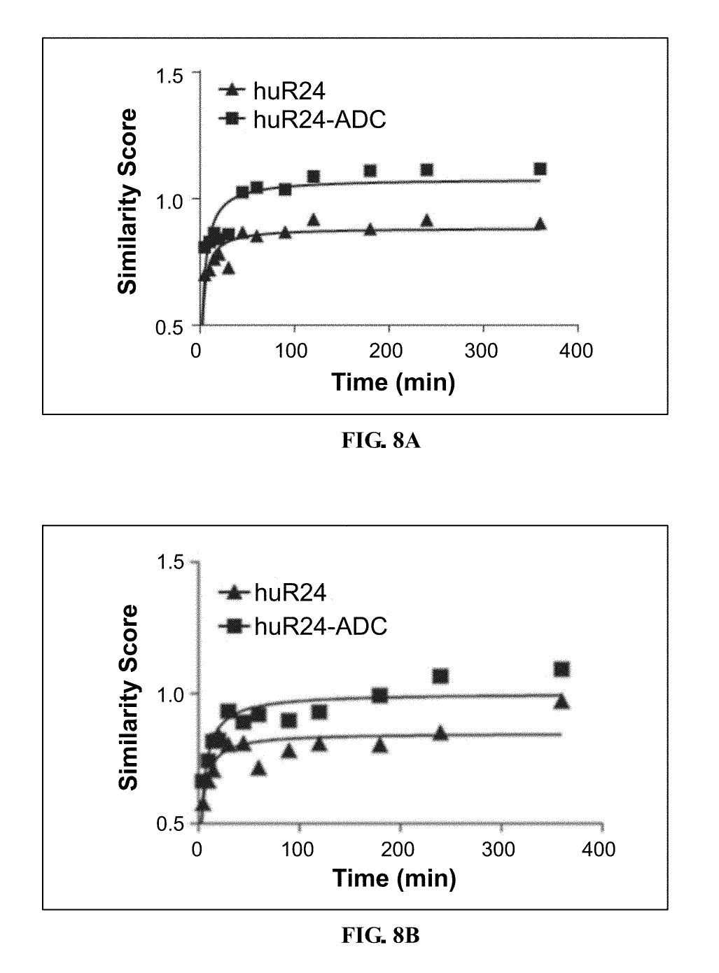

To determine whether huR24 could be used as a potential GD3-ADC, the internalization of the GD3-ADC was examined. An imaging flow cytometry based method to measure internalization was used to determine the internalization of ADC molecules into the melanoma cells. HuR24 naked antibody was tested alongside huR24-ADC in two human melanoma cell lines, Malme-3M cells (FIG. 8A) and SK-MEL-28 cells (FIG. 8B), for the ability to bind cell surface GD3 and be internalized into the cell.

HuR24 and huR24-ADC were added to cells and incubated at 37° C. to start the internalization time course. At each time point, a sample was collected using cell dissociation buffer (Gibco® Catalog number 13151014), the cell sample washed twice with ice cold PBS, re-suspended with 50 μL of 1% paraformaldehyde/versene to stop internalization, and transferred to a 96 well plate. Negative control internalization samples were kept at 4° C. throughout to prevent internalization. Samples were analyzed by an Amnis imaging flow cytometer ImageStream MK II at 40×, using INSPIRE software. Single cells were gated, and 3,000 GD3+ cells were collected from each sample. Membrane and intracellular fluorescent intensity for each sample were determined using Amnis IDEAS software. To derive an endocytic rate constant (Kₑ), the method of Opresko and Wiley (1987) was applied to the membrane and intracellular intensity data from each sample.

The membrane intensity was calculated for each time point and plotted against the intracellular intensity for the linear portions of the time course. The slope of the linear regression provided the endocytic rate constant (Kₑ). To quantitate co-localization between internalized anti-GD3 and the lysosome, samples were incubated with huR24 or huR24-ADC, stained with a fluorescently labeled anti-LAMP-1 that localizes the lysosomal marker LAMP-1, and then acquired using an Amnis imaging flow cytometer. Amnis IDEAS software's “similarity” algorithm was applied to measure the degree of spatial co-localization of the fluorescent signals from the anti-GD3 and anti-LAMP-1 (lysosomal marker) antibodies.

Co-localization of the GD3-antibody and the GD3-ADC with the LAMP-1 lysosomal marker proceeded with indistinguishable kinetics based on a calculated similarity score (FIG. 8A and FIG. 8B). Similarity score, mapped as the log transformed Pearson's Correlation Coefficient, is a measure of the degree to which two images are linearly correlated on a pixel by pixel basis within a region of the cell. A Similarity Score of 0 indicates no internalization because the fluorescence outside of the cell is equal to the fluorescence inside of the cell. A higher Similarity Score suggests increased lysosomal trafficking and rapid internalization, when compared to the comparator antibody. A Similarity Score of 1 or above indicates complete internalization.

After 3 to 4 hours (FIGS. 8A and 8B), huR24 appeared in discrete compartments, co-localized with a marker of lysosomes (Lamp-1). HuR24-ADC consistently had a higher Similarity Score than huR24 over a time period of 5 to 6 hours (FIGS. 8A and 8B), indicating increased lysosomal trafficking and more rapid internalization of huR24-ADC when compared to huR24. The Similarity Score of huR24 was about 0.7 to about 0.8 at times of 100, 200, 300 and 350 minutes. The Similarity Score of huR24-ADC was about 0.9 to about 1.1 at time of 100, 200, 300, and 350 minutes.

These data suggest that huR24 binds cell surface GD3 and is internalized and capable of delivering a cytotoxic agent to a cell expressing GD3, further indicating that huR24-ADC is a potential therapeutic for treating GD3-expressing tumors. Surprisingly, huR24-ADC internalizes substantially better than the huR24 antibody alone. Thus, the antibody alone is not an accurate predictor of the ability of huR24 to be used as a ADC therapeutic and the huR24-ADC is a novel surprising potential ADC therapeutic for a disease, disorder or condition mediated by or associated with GD3 expression on a cell.

### Example 6 Conjugation and Purification Anti-GD3 Antibody-Drug Conjugates

The anti-GD3 antibody drug conjugate (ADC) is prepared via partial reduction of the mAb with tris(2-carboxyethyl)phosphine (TCEP) followed by reaction of reduced cysteine residues with the desired maleimide terminated linker-payload. In particular, anti-GD3 mAb is partially reduced via addition of 2.4 molar excess of tris(2-carboxyethyl)phosphine (TCEP) in 100 mM HEPES (4-(2-hydroxyethyl)-1-piperazineethanesulfonic acid buffer), pH 7.0 and 1 mM diethylenetriaminepentaacetic acid (DTPA) for 2 h at 37° C. The linker-payload maleimidocapronic-auristatin (mcValCitPABC-Aur101) was then added to the reaction mixture at a linker-payload/mAb molar ratio of 7 and reacted for an additional 1 h at 25° C. in the presence of 15% v/v of dimethylacetamide (DMA). After the 1 h incubation period, 3-fold excess of N-ethylmaleimide (NEM) was added to cap the unreacted thiols and was allowed to react for 15 minutes, followed by addition of 6-fold excess L-Cys to quench any unreacted linker-payload. The reaction mixture was dialyzed overnight at 4° C. in phosphate buffered saline (PBS), pH 7.4, and purified via SEC (AKTA avant, Superdex 200). The ADC was further characterized via size exclusion chromatography (SEC) for purity, hydrophobic interaction chromatography (HIC), and liquid chromatography electrospray ionization tandem mass spectrometry (LC-ESI MS) to calculate drug-antibody ratio (DAR). The protein concentration was determined via UV spectrophotometer.

The control ADC contained a non-targeted control TT-21 human IgG1 that was conjugated in the same manner as huR24 antibody. TT-21 is a fully human antibody against tetanus toxoid and has a normal mAb half-life in vivo and is negative for non-specific human or monkey tissue cross-reactivity. HuR24-ADC and the control ADC exhibited similar biophysical profiles. The average DAR of both huR24-ADC and the control ADC was 4.

### Example 7 In Vitro Cytotoxicity of Anti-GD3 ADCs

The anti-tumor effects of huR24-ADC were assessed in a variety of human metastatic melanoma cell lines. Cells were incubated with serial dilutions of huR24-mcValCitPABC-Aur101 or control ADC for 96 hours, at which point cell viability was determined by measuring total cellular ATP levels.

TABLE 8
In vitro cytotoxicity assay EC₅₀ values in μg/mL
on a panel of human melanoma cell lines

| Human Melanoma Cell | | huR24-mcValCitPABC- |
|---|---|---|
| Line | Control ADC | Aur101 |
| G361 | >30 | 3.6 |
| M19-MEL | >30 | 11.5 |
| Malme3M | >30 | 3.5 |
| MeWo | >30 | 4.1 |
| NCI-M14 | >30 | 11.5 |
| SK-MEL-19 | >30 | 1.7 |
| SK-MEL-2 | >30 | 9.2 |
| SK-MEL-28 | >30 | 1.1 |
| SK-MEL-30 | >30 | 4.0 |
| SK-MEL-5 | >30 | 2.6 |
| UACC-257 | >30 | 8.7 |
| UACC-62 | >30 | 15.0 |
| UCSD-242L | >30 | 4.1 |
| UCSD354L | >30 | 11.7 |
| COLO-205* | >30 | >30 |

COLO-205 cells do not express GD3, and were used as an additional control in this experiment. Except for COLO-205, the EC50 is much lower for GD3-ADC compared w control ADC. In all cells tested, which express surface GD3, huR24-ADC was a highly selective cytotoxic that selectively kills metastatic human melanoma cells indicating it is a potential novel therapeutic for that disease.

### Example 8: Cytotoxicity of huR24-ADC in Primary and Commercial Human and Cynomolgus Cell Lines

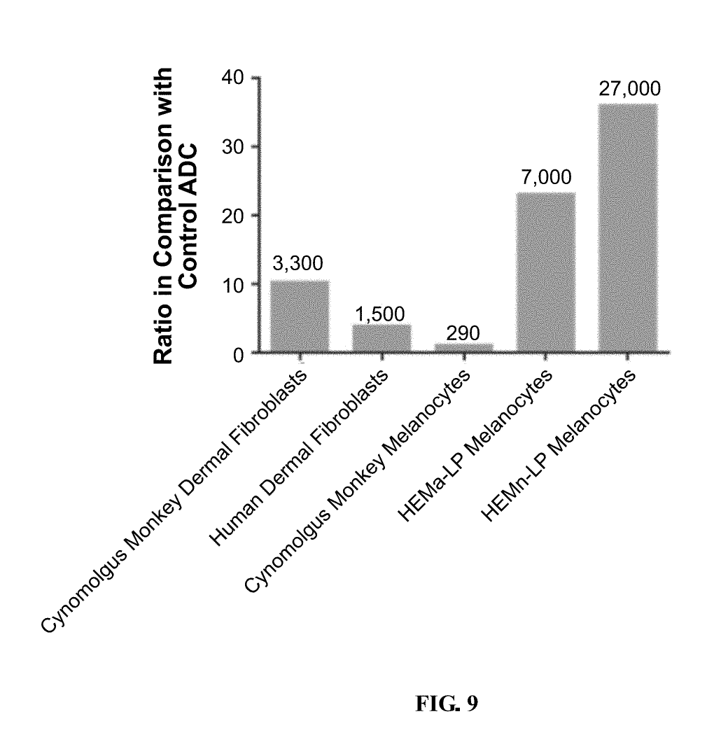

The in vitro binding and cytotoxicity profiles of huR24-mcValCitPABC-Aur101 in primary cynomolgus monkey melanocytes, cynomolgus monkey dermal fibroblasts, and human dermal fibroblasts, in comparison to commercially-available human epidermal melanocytes, were evaluated. Adult human epidermal melanocytes (HEMa-LP) and neonatal human epidermal melanocytes (HEMn-LP) express GD3 at a higher level than cynomolgus monkey melanocytes and cynomolgus monkey and human fibroblasts. The control ADC selected was a negative control isotype-matched Aur101 ADC. Using flow cytometry, and compared with the negative control ADC, a higher number of huR24-ADC molecules bound to dermal fibroblasts and melanocytes obtained from both cynomolgus monkeys and humans, which was interpreted as representing binding to GD3 (FIG. 9). Of the cell types evaluated, huR24-ADC bound to cynomolgus monkey melanocytes at the lowest level (FIG. 9). Further, a higher number of huR24-ADC molecules bound to HEMa-LP and HEMn-LP than bound to Cyno fibroblasts and melanocytes and human fibroblasts (FIG. 9). This was consistent with the higher level of GD3 expression on the surface of HEMa-LP and HEMn-LP cells. The control ADC showed very low potency and therefore EC₅₀ values were not generated. Neither molecule inhibited the growth of the GD3-negative cell line COLO-205 (Table 8).

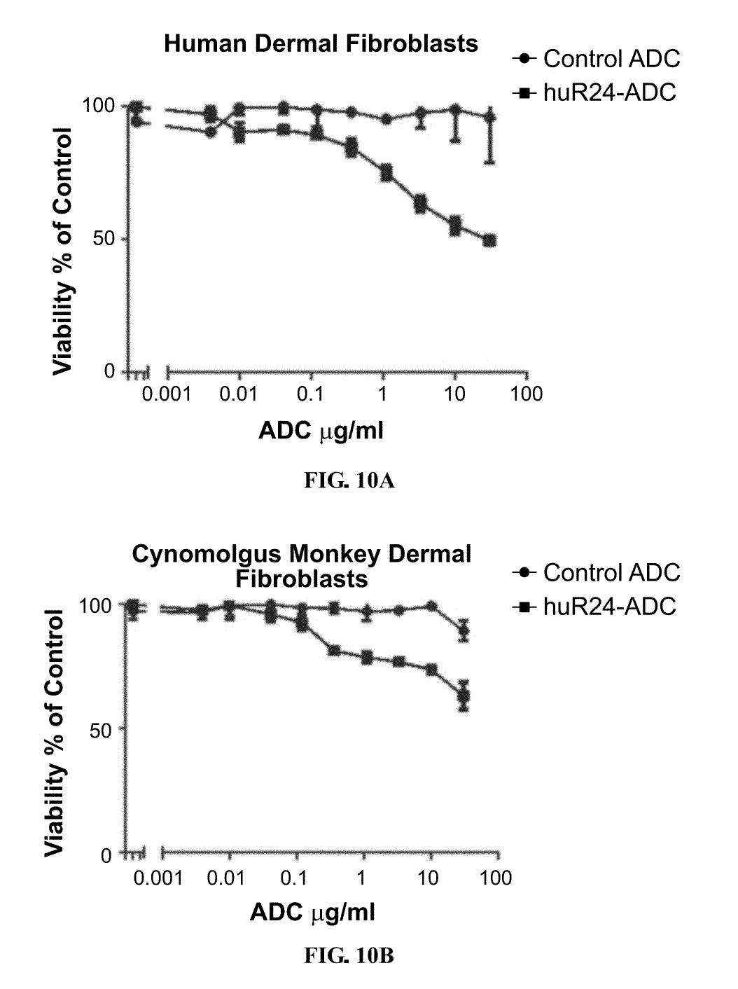

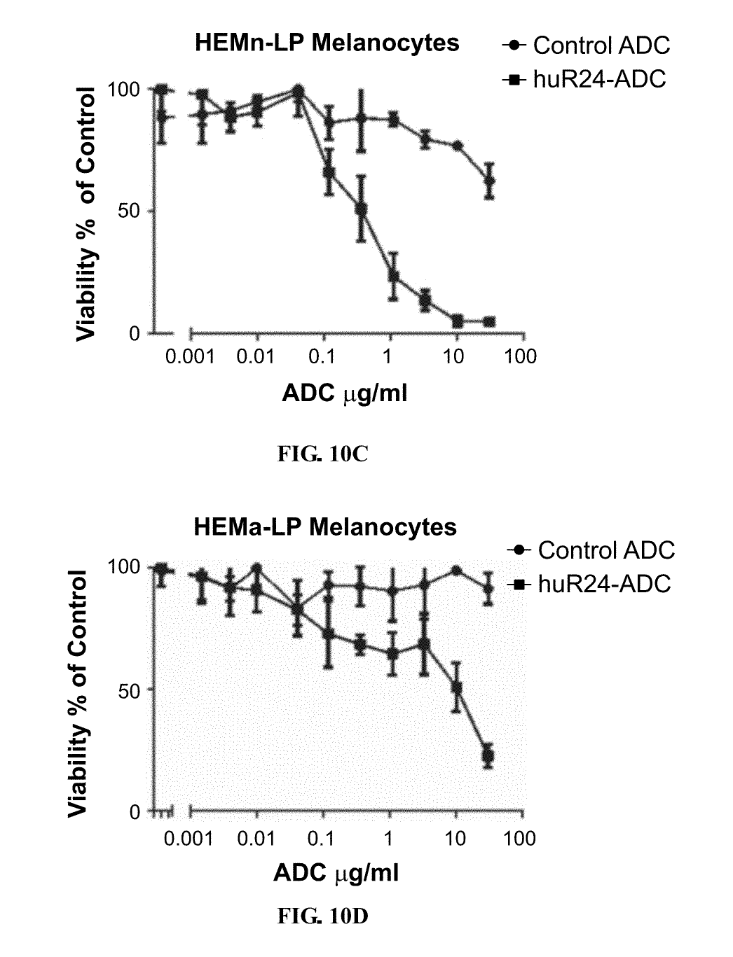

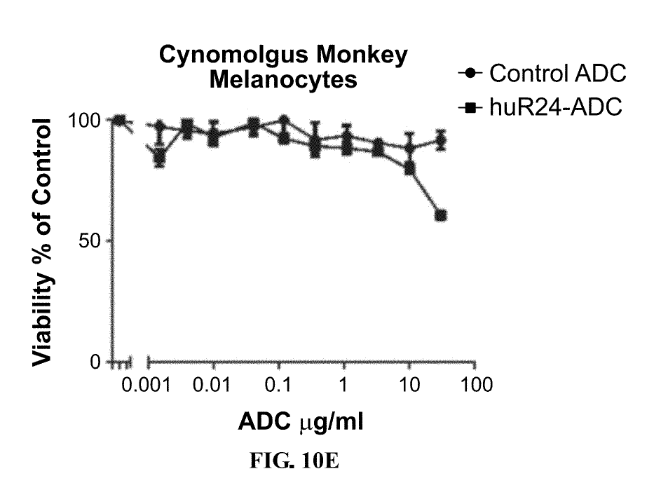

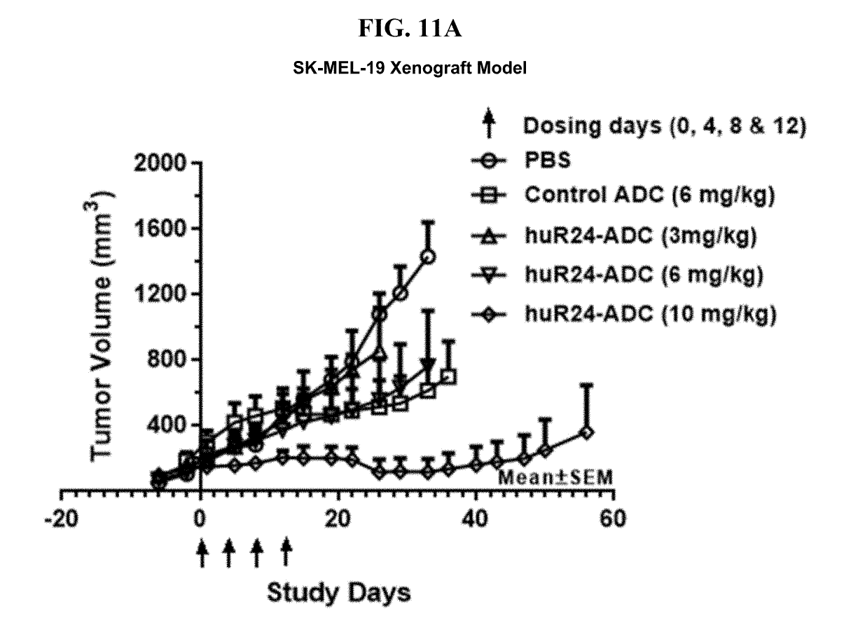

In dermal fibroblasts, the huR24-ADC binding was similar between cynomolgus monkey cells and human cells, and a similar cytotoxicity profile in response to huR24-ADC was observed (FIGS. 10A and 10B). In human epidermal melanocytes, huR24-ADC showed markedly increased cell killing (FIGS. 10C and 10D). In cynomolgus monkey melanocytes, huR24-ADC showed also showed cell killing (FIG. 10E).

huR24-ADC showed cell killing in a concentration-dependent manner. huR24-ADC cell killing was also correlated with the level of GD3 expression on the cell surface. These data confirm that huR24-ADC was a highly selective cytotoxic agent that selectively kills cells which express surface GD3, indicating it is a potential novel therapeutic for that disease, as demonstrated in Example 7 above.

### Example 9 In Vivo Efficacy of Anti-GD3 Antibody-Drug Conjugates

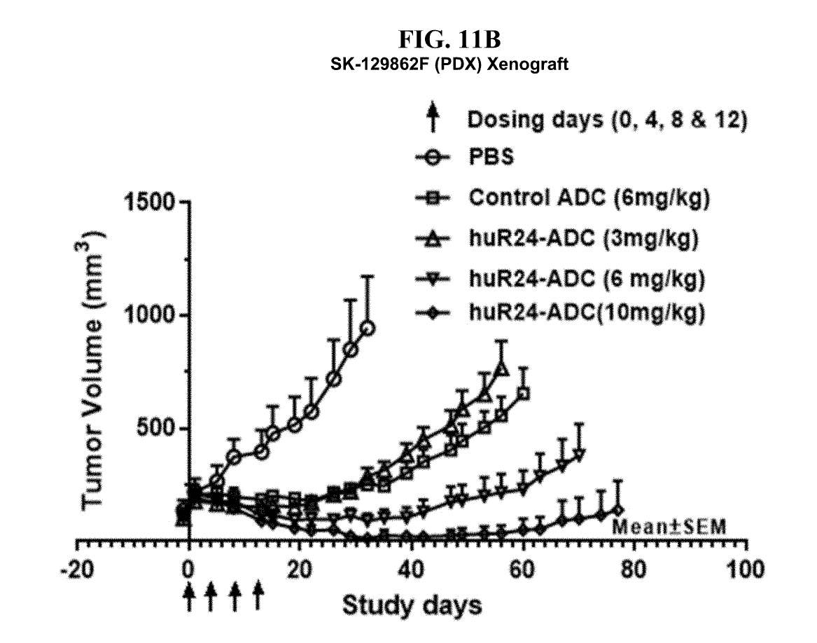

The in vivo pharmacology of huR24-ADC was evaluated by assessing anti-tumor activity in two metastatic melanoma human tumors: a cell line-derived xenograft model (SK-MEL-19; FIG. 11A) and a patient-derived xenograft (PDX) (129862F-PDX; FIG. 11B). The models were selected based on GD3 expression by flow cytometry and immunohistochemistry (IHC), and on the ability to form tumors in female athymic nu/nu mice or female NSG (Non-obese diabetic severely combined immune-deficient mice with no IL-2 gamma receptor) mice. For both of the models described herein, huR24-ADC was evaluated at 3, 6, and 10 mg/kg dose levels and compared directly with a 6 mg/kg dose level of a negative control ADC that carries the same cytotoxic payload (Aur101). In each study, animals were randomized into study groups such that tumor volume cohorts averaged between 200-300 mm³ at the time of first dose. After randomization, animals were dosed IV Q4 days×4 doses. A summary of the results is presented in Table 9. Tumor growth inhibition curves for SK-MEL-19 and SK-129862F (PDX) are depicted in FIGS. 11A and 11B, respectively. Average tumor volumes are shown in Tables 10 and 11.

TABLE 9
Summary of In Vivo Efficacy Studies with huR24-mcValCitPABC-Aur101 and Control ADC

| Tumor | | Dose | Dose | | Dose Level | | % T/C* |
|---|---|---|---|---|---|---|---|
| Model | Study Name | Regimen | Route | Test Article | (mg/kg) | | (day) |
| SK-MEL-19 | GST-LJ-2014-GD3- | Q4d x 4 | IV | huR24- | 3 | mg/kg | 42% (33) |
| | ADC_SKMEL-19 | | | mcValCitPABC- | | | |
| | | | | Aur101 | | | |
| | | Q4d x 4 | IV | huR24- | 6 | mg/kg | 48% (33) |
| | | | | mcValCitPABC- | | | |
| | | | | Aur101 | | | |
| | | Q4d x 4 | IV | huR24- | 10 | mg/kg | 15% (33) |
| | | | | mcValCitPABC- | | | |
| | | | | Aur101 | | | |
| | | Q4d x 4 | IV | Control ADC | 6 | mg/kg | 37% (33) |
| SF-129862F | GST-LJ-2014-GD3- | Q4d x 4 | IV | huR24- | 3 | mg/kg | 35% (32) |
| | ADC_SK129862F-PDx | | | mcValCitPABC- | | | |
| | | | | Aur101 | | | |
| | | Q4d x 4 | IV | huR24- | 6 | mg/kg | 11% (32) |
| | | | | mcValCitPABC- | | | |
| | | | | Aur101 | | | |
| | | Q4d x 4 | IV | huR24- | 10 | mg/kg | 4% (32) |
| | | | | mcValCitPABC- | | | |
| | | | | Aur101 | | | |
| | | Q4d x 4 | IV | Control ADC | 6 | mg/kg | 25% (32) |

IV = Intravenous;
mg/kg = milligrams per kilogram;
Q4d x 4 = Dose every 4 days for 4 cycles;
% T/C = Percentage of relative tumor volume in test groups over control groups;
*% T/C was calculated on the day when Saline group was terminated.

In the SK-MEL-19 melanoma model, huR24-ADC inhibited tumor growth at the 10 mg/kg dose and showed a trend toward slower growth of tumors at 3 and 6 mg/kg, compared with the group administered saline. The average tumor volumes in animals administered 10 mg/kg showed a statistically significant reduction on Day 33 relative to animals administered saline (the final day before the vehicle cohort was euthanized). At this time point, tumor volumes of the groups administered huR24-mcValCitPABC-Aur101 relative to the saline vehicle were 42%, 48%, and 15% at 3, 6 and 10 mg/kg, respectively. huR24-mcValCitPABC-Aur101 at the 10 mg/kg dose resulted in long-term tumor regression, with two mice showing no palpable tumor at 56 days after initiation of drug administration. The tumor growth curves for each of the cohorts were plotted up to the day when at least one animal was found dead or was euthanized based on morbidity or tumor burden. No significant body weight differences were noted among any dose cohorts.

TABLE 10
Summary of Average Tumor Volumes
in the SK-MEL-19 Xenograft Model

| | PBS | Control | | | |
|---|---|---|---|---|---|
| | 0.1 ml/ | ADC | huR24-ADC | | |
| Day | 10 g bw | 6 mg/kg | 3 mg/kg | 6 mg/kg | 10 mg/kg |
| −6 | 101 ± 20 | 216 ± 47 | 108 ± 38 | 120 ± 17 | 147 ± 19 |
| −2 | 161 ± 25 | 344 ± 65 | 161 ± 57 | 158 ± 31 | 182 ± 24 |
| 1 | 207 ± 45 | 481 ± 118 | 202 ± 71 | 255 ± 88 | 170 ± 26 |
| 5 | 271 ± 84 | 526 ± 116 | 274 ± 97 | 277 ± 91 | 205 ± 29 |
| 8 | 287 ± 96 | 579 ± 114 | 336 ± 119 | 353 ± 69 | 173 ± 34 |
| 12 | 479 ± 132 | 549 ± 105 | 429 ± 152 | 427 ± 75 | 207 ± 42 |
| 15 | 564 ± 65 | 550 ± 113 | 556 ± 196 | 511 ± 121 | 286 ± 51 |
| 19 | 683 ± 60 | 576 ± 113 | 638 ± 226 | 550 ± 145 | 286 ± 51 |
| 22 | 791 ± 46 | 723 ± 115 | 742 ± 262 | 601 ± 181 | 272 ± 61 |
| 26 | 1080 ± 127 | 753 ± 116 | 859 ± 304 | 838 ± 193 | 275 ± 84 |
| 29 | 1210 ± 164 | 861 ± 108 | | 948 ± 199 | 287 ± 87 |
| 33 | 1434 ± 210 | 982 ± 144 | | 1146 ± 266 | 278 ± 88 |
| 36 | | | | | 320 ± 106 |
| 40 | | | | | 380 ± 123 |
| 43 | | | | | 421 ± 137 |
| 47 | | | | | 469 ± 160 |
| 50 | | | | | 590 ± 219 |
| 56 | | | | | 846 ± 350 |

Tumor volumes are measured in mm³

TABLE 11
Summary of Average Tumor Volumes in
the SK-129862F(PDX) Xenograft Model

| | PBS | Control | | | |
|---|---|---|---|---|---|
| | 0.1 ml/ | ADC | huR24-ADC | | |
| Days | 10 g bw | 6 mg/kg | 3 mg/kg | 6 mg/kg | 10 mg/kg |
| −1 | 138 ± 45 | 115 ± 20 | 104 ± 39 | 110 ± 18 | 118 ± 31 |
| 1 | 220 ± 59 | 206 ± 28 | 179 ± 68 | 188 ± 32 | 208 ± 46 |
| 5 | 266 ± 71 | 214 ± 25 | 168 ± 64 | 176 ± 27 | 195 ± 52 |
| 8 | 374 ± 79 | 198 ± 39 | 159 ± 60 | 178 ± 41 | 148 ± 50 |
| 13 | 396 ± 97 | 186 ± 20 | 152 ± 58 | 115 ± 23 | 91 ± 22 |
| 15 | 480 ± 121 | 202 ± 18 | 159 ± 60 | 117 ± 23 | 81 ± 24 |
| 19 | 517 ± 123 | 190 ± 21 | 152 ± 58 | 94 ± 20 | 59 ± 20 |
| 22 | 576 ± 146 | 180 ± 15 | 171 ± 65 | 100 ± 28 | 48 ± 24 |
| 26 | 721 ± 173 | 216 ± 17 | 208 ± 79 | 97 ± 20 | 52 ± 22 |
| 29 | 851 ± 220 | 233 ± 20 | 222 ± 84 | 114 ± 32 | 23 ± 12 |
| 32 | 946 ± 229 | 252 ± 19 | 288 ± 109 | 91 ± 32 | 13 ± 13 |
| 35 | | 247 ± 34 | 316 ± 119 | 104 ± 38 | 26 ± 26 |
| 39 | | 303 ± 50 | 386 ± 146 | 105 ± 45 | 23 ± 23 |
| 42 | | 353 ± 54 | 451 ± 170 | 130 ± 55 | 21 ± 21 |
| 47 | | 406 ± 65 | 515 ± 195 | 175 ± 66 | 26 ± 26 |
| 49 | | 445 ± 74 | 589 ± 223 | 182 ± 69 | 30 ± 30 |
| 53 | | 505 ± 72 | 653 ± 247 | 202 ± 82 | 33 ± 33 |
| 56 | | 556 ± 84 | 769 ± 291 | 217 ± 81 | 36 ± 36 |
| 60 | | 656 ± 114 | | 231 ± 84 | 50 ± 50 |
| 63 | | | | 288 ± 100 | 55 ± 55 |
| 67 | | | | 334 ± 118 | 94 ± 88 |
| 70 | | | | 381 ± 139 | 100 ± 93 |
| 74 | | | | | 117 ± 108 |
| 77 | | | | | 139 ± 130 |

Tumor volumes are measured in mm³

HuR24-ADC significantly reduced the growth of SK-129862F PDX implanted in female NSG mice when administered huR24-ADC at doses of 3, 6, or 10 mg/kg in a Q4d×4 IV regimen. The SK-129862F patient-derived xenografts are from true melanoma cancer samples from patients with minimal expansion on plastic in vitro before they are injected into mice. All groups administered huR24-mcValCitPABC-Aur101 showed tumor volume reductions that were statistically greater compared with the vehicle control group. On Day 32 (the final day before the vehicle cohort was euthanized) the volume of the treatment group tumors relative to the vehicle were 35%, 11% and 4% at huR24-ADC doses of 3, 6 and 10 mg/kg, respectively. The isotype control ADC, administered at 6 mg/kg, showed tumor volume reduction similar to a dose of 3 mg/kg of huR24-ADC, but did not reduce the tumor growth to the same extent as an equivalent dose of huR24-ADC and 10 mg/kg huR24-ADC showed much greater reduction in tumor volume compared with the control ADC demonstrating that the tumor reducing effect is selective for GD3-expressing tumor cells. The tumor growth curves for each of the cohorts were plotted up to the day when at least an animal was found dead or euthanized based on morbidity or tumor burden. Interestingly, while control treated animals had a slight decrease in body weight as tumors grew, animals treated with either 6 or 10 mg/kg had a substantial increase in body weight while tumors were regressing, demonstrating a correlation between weight gain in mice and regression of tumor burden.

### Example 10 Pharmacokinetics and Toxicokinetics of Anti-GD3 Antibody Drug Conjugates in Mice, Rats, and Cynomolgus Monkeys

The pharmacokinetic parameters of huR24 and huR24-ADC are presented in Tables 12 and 13 below. The pharmacokinetics of huR24 and huR24-ADC were determined in female athymic nu/nu mice (4/time point/dose group) after a single IV administration of huR24-ADC at 6 or 10 mg/kg. Following IV administration of huR24-ADC to female mice, systemic exposure of huR24-ADC, as assessed by maximum plasma concentration (Cₘₐₓ) and area under the concentration-time curve (AUCₗₐₛₜ), increased with an increase in dose from 6 to 10 mg/kg. huR24 ADC exhibited a low plasma clearance (CL; 0.8 mL/hour/kg) and low steady-state volume of distribution (Vₛₛ; 0.13 L/kg) for both dose groups. The apparent t_{1/2} of huR24-ADC was 5.9 and 5.6 days, for the 6 and 10 mg/kg dose groups, respectively. Following IV administration of huR24 to female mice, systemic exposure of huR24 as assessed by Cₘₐₓ and AUCₗₐₛₜ, increased with an increase in dose of huR24 from 6 to 10 mg/kg. huR24 exhibited a similarly low plasma CL (0.4 mL/hour/kg) at each dose and low Vss (0.14 to 0.15 L/kg). The apparent t_{1/2} for huR24 following dosing with huR24 was 10.9 and 10.6 days, for the 6 and 10 mg/kg dose groups, respectively.

The pharmacokinetics of huR24 and huR24-ADC were determined in male Wistar Han rats (4/time point/dose group) after single IV administration of huR24 or huR24-ADC at 6 or 30 mg/kg. Following IV administration of huR24-ADC to male rats, systemic exposure of huR24-ADC, as assessed by the extrapolated concentration at time zero (C₀) and AUCₗₐₛₜ, increased with an increase in dose from 6 to 30 mg/kg. huR24-ADC exhibited a low serum plasma clearance (CL; 0.5 mL/hour/kg) at each dose and a low Vₛₛ (0.10 to 0.094 L/kg). The apparent t_{1/2} for huR24-ADC was 8.5 and 8.1 days, for the 6 and 30 mg/kg dose groups, respectively. Following IV administration of huR24 to male rats, systemic exposure of huR24, as assessed by C₀ and AUCₗₐₛₜ, increased with an increase in huR24-ADC dose from 6 to 30 mg/kg as well. huR24 also exhibited a low serum CL (0.2 mL/hour/kg) and low Vss (0.094 to 0.096 L/kg). The apparent t_{1/2} for huR24 following dosing with huR24 was similar across dose groups at 13.7 and 12.3 days, for the 6 and 30 mg/kg dose groups, respectively.

As part of a 46-day exploratory toxicity study, the pharmacokinetics of huR24 and huR24-ADC were determined on day 22 after repeat IV (bolus) administration (3 cycles, once every 3 weeks) of huR24 or huR24-ADC at doses of 3, 6, or 15 mg/kg/dose to male and female cynomolgus monkeys (1 or 2/sex/dose group). Systemic exposure of huR24-ADC, based on Cmax and AUC, increased with increases in dose from 3 to 6 to 15 mg/kg/dose. huR24-ADC exhibited a low serum plasma clearance (CL; 0.3 to 0.4 mL/hour/kg), a low Vss (0.056 to 0.068 L/kg), and an apparent t_{1/2} from 7.0 to 7.7 days. Systemic exposure of huR24, based on Cₘₐₓ and AUC, increased with increases in huR24 dose from 3 to 6 to 15 mg/kg/dose. huR24 exhibited a similarly low serum CL (approximately 0.2 mL/hour/kg), low Vₛₛ (0.051 to 0.067 L/kg) and an apparent t_{1/2} from 10.8 to 16 days.

TABLE 12
Mean Pharmacokinetics of huR24-ADC in Mice, Rats, and Cynomolgus
Monkeys Following IV Administration of huR24-ADC

| | Dose | Cₘₐₓ | AUCₗₐₛₜ | T_{1/2} |
|---|---|---|---|---|
| Species | (mg/kg/dose) | (μg/mL) | (μg · hour/mL) | (days) |
| Mouse | 6 | 116 | 6510 | 5.9 |
| | 10 | 203 | 10600 | 5.6 |
| Rat | 6 | 156 | 11500 | 8.5 |
| | 30 | 952 | 57600 | 8.1 |
| Monkey | 3 | 105 | 7990 | 7.7 |
| | 6 | 217 | 15800 | 7.6 |
| | 15 | 554 | 45700 | 7.0 |

TABLE 13
Mean Pharmacokinetics of huR24 in Mice, Rats, and Cynomolgus
Monkeys Following IV Administration of huR24

| | Dose | Cₘₐₓ | AUCₗₐₛₜ | T_{1/2} |
|---|---|---|---|---|
| Species | (mg/kg/dose) | (μg/mL) | (μg · hour/mL) | (days) |
| Mouse | 6 | 109 | 9290 | 10.9 |
| | 10 | 166 | 14200 | 10.6 |
| Rat | 6 | 130 | 19000 | 13.7 |
| | 30 | 903 | 88100 | 12.3 |
| Monkey | 3 | 99.1 | 13800 | 16.0 |
| | 6 | 190 | 26400 | 13.0 |
| | 15 | 520 | 75300 | 10.8 |

### Example 11 Pharmacokinetics and Dosing of Anti-GD3 Antibody-Drug Conjugates

As part of a 6-week pivotal toxicity study, the plasma concentrations of huR24 and huR24-ADC were determined on Days 1 and 22 after repeat IV (bolus) administration (3 cycles: Days 1, 22 and 43) of huR24-ADC at doses of 6, 9, or 12 mg/kg/dose to male and female cynomolgus monkeys (3 or 5/sex/dose group). In general, there were no sex-related differences in exposure for each analyte across the dose groups and the mean maximum observed drug concentration in plasma (Cₘₐₓ) and the mean area under concentration-time curve from 0 to 504 hours post dose (AUC₅₀₄) values on Day 1 were similar to the values of each analyte on Day 22, with exposure increasing as the dose of huR24 increased on Day 22. As assessed by mean AUC₅₀₄ values, exposure of huR24 was higher compared with exposure of huR24-ADC, with mean ratios (AUC₅₀₄, huR24/huR24-ADC) ranging from 1.6 to 2.3 across dose groups on Days 1 and 22. The pharmacokinetic parameters are provided in Table 14.

Human predicted PK parameters for huR24-ADC were scaled from cynomolgus monkey pharmacokinetics from the 6-week pivotal toxicity study, using a scaling factor of 1.0 for clearance and for volume. In the present clinical translation approach, it is assumed that plasma huR24-ADC concentrations are driving efficacy and mouse PK/PD parameters translate directly to human. The pharmacokinetics of huR24-ADC in humans are expected to be linear with a projected plasma clearance (CL) of 0.375 mL/h/kg, a volume of distribution at steady state (Vₛₛ) of approximately 0.086 L/kg, and a terminal elimination half-life (t_{1/2}) of 7 days. The predicted terminal elimination half-life (t_{1/2}) of 7 days in humans is consistent with the terminal elimination half-life measure in monkeys as described in Example 9 and Table 12 above.

For calculations of safety margins, the predicted average concentration (Cₐᵥ) is 2.3 μg/mL and the predicted maximum observed drug concentration in plasma (Cₘₐₓ) is 18.4 μg/mL at a proposed starting dose of 0.5 mg/kg of huR24-ADC.

TABLE 14
Summary of the Pharmacokinetic Data for huR24 and huR24-ADC

| Study | Dose | Cₘₐₓ (μg/mL) | | AUC₅₀₄(μg · hour/mL) | |
|---|---|---|---|---|---|
| Moiety | (mg/kg/dose) | Day 1 | Day 22 | Day 1 | Day 22 |
| huR24 | 6 | 175 | 233 | 23700 | 37400 |
| | 9 | 238 | 309 | 38600 | 48500 |
| | 12 | 368 | 388 | 51800 | 73600 |
| huR24-ADC | 6 | 229 | 213 | 15000 | 16500 |
| | 9 | 304 | 391 | 20000 | 25800 |
| | 12 | 435 | 383 | 28500 | 33300 |

Considered together, Examples 10 and 11 show that exposure of huR24-ADC in animals was found to be roughly dose proportional across the range of pharmacokinetic parameters tested (See Tables 12 and 13 below). These data suggest that there are no unusual pharmacodynamic effects, such as a drug sink or severe anti-drug antibodies clearing huR24-ADC, effecting huR24-ADC. In conclusion, these data suggest that huR24-ADC may be a safe and effective potential therapeutic to treat tumors expressing GD3.

### Example 12 A Phase 1 Dose Escalation Study of an Anti-GD3 Antibody-Drug Conjugate

A Phase 1 dose escalation, dose expansion, safety, pharmacokinetic study enrolled sequential cohorts of adult patients with unresectable Stage III or Stage IV melanoma who have progressed or have not tolerated prior therapy. HuR24-ADC, starting at 0.5 mg/kg, was infused intravenously over approximately 60 minutes. The dosing regimen repeats every 21 days. The phase one study includes two parts.

Part 1, dose escalation, of the trial includes up to 20 patients and will estimate the Maximum Tolerated Dose (MTD)/Recommended Phase 2 Doses (RP2D) using a Bayesian dose escalation schedule. Part 1 of the trial is ongoing. All patients dosed to date tolerated huR24 ADC.

Part 2 will be a dose expansion cohort of up to 20 patients with unresectable stage III or IV melanoma enrolled at the RP2D. However, patients treated in Part 1 at what eventually will be determined to be the RP2D will be included in this cohort of 20 patients. The goal of Part 2 is to confirm safety and tolerability and to explore preliminary evidence of antitumor effects of huR24-ADC.

Secondary endpoints include pharmacokinetics and immunogenicity assessments.

Treatment with the huR24-ADC will continue until disease progression, patient refusal or unacceptable toxicity occurs. Patients who demonstrate clinical benefit with manageable toxicity and are willing to continue receiving huR24-ADC will be given the opportunity to remain on study.

The invention thus has been disclosed broadly and illustrated in reference to representative embodiments described above. Those skilled in the art will recognize that various modifications can be made to the present invention without departing from the spirit and scope thereof. All publications, patent applications, and issued patents, are herein incorporated by reference to the same extent as if each individual publication, patent application or issued patent were specifically and individually indicated to be incorporated by reference in its entirety. Definitions that are contained in text incorporated by reference are excluded to the extent that they contradict definitions in this disclosure.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. In particular, any aspect of the invention described in the claims, alone or in combination with one or more additional claims and/or aspects of the description, is to be understood as being combinable with other aspects of the invention set out elsewhere in the claims and/or description and/or sequence listings and/or drawings.

Although the disclosed teachings have been described with reference to various applications, methods, and compositions, it will be appreciated that various changes and modifications can be made without departing from the teachings herein and the claimed invention below. The examples are provided to better illustrate the disclosed teachings and are not intended to limit the scope of the teachings presented herein. While the present teachings have been described in terms of these exemplary embodiments, numerous variations and modifications of these exemplary embodiments are possible without undue experimentation. All such variations and modifications are within the scope of the current teachings.

Where aspects or embodiments of the invention are described in terms of a Markush group or other grouping of alternatives, the present invention encompasses not only the entire group listed as a whole, but each member of the group individually and all possible subgroups of the main group, but also the main group absent one or more of the group members. The present invention also envisages the explicit exclusion of one or more of any of the group members in the claimed invention.

All references cited herein, including patents, patent applications, papers, text books, and the like, and the references cited therein, to the extent that they are not already, are hereby incorporated by reference in their entirety. In the event that one or more of the incorporated literature and similar materials differs from or contradicts this application, including but not limited to defined terms, term usage, described techniques, or the like, this application controls.

The description and examples detail certain specific embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

1. An antibody, or antigen-binding fragment thereof, that specifically binds GD3, comprising:
(i) a heavy chain variable region (VH) that comprises:
(a) a VH complementarity determining region 1 (CDR-H1) comprising the amino acid sequence of SEQ ID NO: 2,
(b) a VH CDR-H2 comprising the amino acid sequence of SEQ ID NO: 4; and
(c) a VH CDR-H3 comprising the amino acid sequence of SEQ ID NO: 6; and
(ii) a light chain variable region (VL) that comprises:
(a) a VL CDR-L1 comprising the amino acid sequence of SEQ ID NO: 10,
(b) a VL CDR-L2 comprising the amino acid sequence of SEQ ID NO: 12; and
(c) a VL CDR-L3 comprising the amino acid sequence of SEQ ID NO: 13,
wherein the VH comprises a VL framework sequence and a VH framework sequence, and (i) wherein the VL framework sequence is at least 98%, 99%, or 100% identical to a DPK9 human germline framework sequence from which it is derived, and (ii) wherein the VH framework sequence is at least 91%, 92%, 93%, 94%, 95%, 96%, 97%, 98%, 99%, or 100% identical to a DP54 human germline framework sequence from which it is derived.

2. The antibody, or antigen binding fragment thereof, of claim 1, comprising (i) a VH comprising the amino acid sequence of SEQ ID NO: 1, and (ii) a VL comprising the amino acid sequence of SEQ ID NO: 9.

3. The antibody or antigen binding fragment thereof, of claim 2, comprising a VH having an amino acid sequence that is 90% identical to SEQ ID NO: 1 or a VL having an amino acid sequence that is at least 90% identical to SEQ ID NO: 9.

4. The antibody, or antigen binding fragment thereof, of claim 2, comprising the VH sequence encoded by nucleic acid sequence of the insert in the plasmid deposited at the ATCC and having ATCC Accession No. PTA-124057, and the VL sequence encoded by nucleic acid sequence of the insert in the plasmid deposited at the ATCC and having ATCC Accession No. PTA-124058.

5. An antibody, or antigen binding fragment thereof, that competes for binding to GD3 with the antibody, or antigen-binding fragment thereof, of claim 1.

6. The antibody, or antigen binding fragment thereof, of claim 1, comprising an Fc domain, wherein the Fc domain is the Fc domain of an IgA₁ IgA₂, IgD, IgE, IgM, IgG₁, IgG₂, IgG₃, or IgG₄.

7. An antibody, or antigen binding fragment thereof, comprising a heavy chain set forth as SEQ ID NO: 7 and a light chain set forth as SEQ ID NO: 14.

8. An isolated nucleic acid molecule, comprising one or more nucleotide sequences encoding the antibody, or antigen binding fragment thereof, of claim 1.

9. A vector comprising the nucleic acid molecule of claim 8.

10. A host cell comprising the nucleic acid molecule of claim 9.

11. An antibody-drug conjugate (ADC) of the formula:
Ab-(L-D)p,
wherein:
(a) Ab is an antibody, or antigen-binding fragment thereof, that specifically binds GD3;
(b) L-D is a linker-drug moiety, wherein L is a linker, and D is a drug;
(c) p is an integer from about 1 to 12.

12. The ADC of claim 11, wherein the Ab comprises:
(i) a heavy chain variable region (VH) that comprises:
(a) a VH CDR-H1 comprising the amino acid sequence of SEQ ID NO: 2,
(b) a VH CDR-H2 comprising the amino acid sequence of SEQ ID NO: 4; and
(c) a VH CDR-H3 comprising the amino acid sequence of SEQ ID NO: 6; and
(ii) a light chain variable region (VL) that comprises:
(a) a VL CDR-L1 comprising the amino acid sequence of SEQ ID NO: 10,
(b) a VL CDR-L2 comprising the amino acid sequence of SEQ ID NO: 12; and
(c) a VL CDR-L3 comprising the amino acid sequence of SEQ ID NO: 13,
wherein the VH comprises a VL framework sequence and a VH framework sequence, and (i) wherein the VL framework sequence is at least 98%, 99%, or 100% identical to a DPK9 human germline framework sequence from which it is derived, and (ii) wherein the VH framework sequence is at least 91%, 92%, 93%, 94%, 95%, 96%, 97%, 98%, 99%, or 100% identical to a DP54 human germline framework sequence from which it is derived.

13. The ADC of claim 12, wherein the Ab comprises (i) a VH comprising the amino acid sequence of SEQ ID NO: 1, and (ii) a VL comprising the amino acid sequence of SEQ ID NO: 9.

14. The antibody drug conjugate of claim 11, comprising an Fc domain, wherein the Fc domain is the Fc domain of an IgA₁ IgA₂, IgD, IgE, IgM, IgG₁, IgG₂, IgG₃, or IgG₄.

15. The ADC of claim 11, wherein the linker comprises mcValCitPABC.

16. The ADC of claim 11, wherein the drug is auristatin 0101.

17. The ADC of claim 11:
Ab-(L-D)p, wherein:
(a) Ab is an antibody comprising a heavy chain set forth as SEQ ID NO: 7 and a light chain set forth as SEQ ID NO: 14;
(b) L-D is a linker-drug moiety, wherein L is a linker, and D is a drug, wherein the linker is mcValCitPABC, and wherein the drug is auristatin 0101; and
(c) p is 4.

18. A process for producing an ADC of claim 11 comprising:
(a) linking the linker to the drug moiety;
(b) conjugating the linker-drug moiety to the antibody; and
(c) purifying the ADC.

19. A pharmaceutical composition comprising the ADC of claim 11 and a pharmaceutically acceptable carrier.

20. A method of treating a disease, disorder or condition associated with or mediated by GD3 cell surface expression in a subject in need thereof, comprising administering a therapeutically effective amount of a composition comprising the ADC of claim 11 to the subject.

21. A method of treating a disease, disorder or condition associated with or mediated by an elevated level of a GD3 activity in a subject in need thereof, comprising administering a therapeutically effective amount of a composition comprising the ADC of claim 11 to the subject.

22. The method of claim 20, wherein the disease, disorder or condition is melanoma, breast cancer, glioma, glioblastoma, or lung cancer.

23. The method of claim 21, wherein the disease, disorder or condition is melanoma, breast cancer, glioma, glioblastoma, or lung cancer.
